(19) United States
(12) Patent Application Publication
(10) Pub. No.: US 2025/0108110 A1
(43) Pub. Date: Apr. 3, 2025

(54) COMBINATION THERAPY FOR USE IN TREATMENT OF NON-SMALL CELL LUNG CANCER

(71) Applicant: Alethia Biotherapeutics Inc., Montréal (CA)

(72) Inventors: Mario FILION, Longueuil (CA); Julie LAURIN, Saint-Lazare (CA); Jacques JOLIVET, Val-David (CA)

(73) Assignee: Alethia Biotherapeutics Inc., Montréal, QC (CA)

(21) Appl. No.: 18/729,922

(22) PCT Filed: Feb. 6, 2023
(86) PCT No.: PCT/CA2023/050151

§ 371 Date: Jul. 18, 2024

Related U.S. Application Data
(60) Provisional application No. 63/307,533, filed on Feb. 7, 2022.
(60) Provisional application No. 63/333,597, filed on Apr. 22, 2022.

(51) Int. Cl. A61K 39/395; A61K 31/337; A61K 39/00; A61P 35/00

(52) U.S. Cl. CPC A61K 39/3955; A61K 31/337; A61P 35/00; A61K 2039/54; A61K 2039/545; A61K 2039/55

(57) ABSTRACT

The present disclosure generally relates to a combination therapy comprising an anti-clusterin antibody or antigen binding fragment thereof and a taxane and its use in the treatment of subjects having non-small cell lung cancer (NSCLC). The combination therapy may be used for treating NSCLC patients including, NSCLC patients that have failed prior treatment that comprises an immune checkpoint antibody, NSCLC patients having one or more lesions showing low expression of programmed death ligand 1 (PD-L1), NSCLC patients having no evidence of PD-L1 expression or NSCLC patients that are not eligible for or would unlikely benefit from treatment comprising an anti-PD-1 or anti-PD-L1 immune checkpoint antibody.

### TECHNICAL FIELD

The present disclosure generally relates to a combination therapy comprising an anti-clusterin antibody or antigen binding fragment thereof and a taxane and its use in the treatment of subjects having non-small cell lung cancer (NSCLC).

### BACKGROUND

The molecular mechanisms responsible for the occurrence of metastatic carcinomas are beginning to be elucidated with the identification of key regulators. Increasing evidence points to tumor cell epithelial-to-mesenchymal (EMT) as an important contributing process to metastatic evolution. The occurrence of EMT during tumor progression permits epithelial tumor cells, that are non-invasive and non-metastatic, to move from the primary tumor, invade the surrounding tissue, enter the bloodstream and finally disseminate to, and proliferate at secondary sites. In addition, epithelial cancer cells that undergo EMT adopt a behavior that is very similar to cancer stem cells (CSCs) including an inherent resistance to chemotherapy and immune evasion (Shibue et al., 2017; Terry et al. 2017).

Despite the short-term effectiveness of first- and second-line therapies such as chemotherapeutic agents and immune checkpoint inhibitors in cancer therapy, a high proportion of subjects become refractory to these therapies due to the resistance of tumor cells to anti-cancer agents and the survival of tumor-initiating cells, two events that ultimately result in an increase in metastasis and poor subject survival.

The mechanism underlying resistance to immune checkpoint inhibitors can vary. However, it is generally accepted that checkpoint inhibitors work best against so-called immunologically hot tumors that is tumors that have been invaded by T cells creating an inflamed tumor. In contrast, immunologically cold tumors are poorly responsive to immunotherapy because for unknown reasons these tumors haven't been recognized or haven't provoke a strong immune response and therefore T cells have not penetrated to tumor or its microenvironment.

Patients who have received a prior first-line immune checkpoint inhibitor as a single agent are offered platinum-based chemotherapy in second-line. Single agent docetaxel can be administered as second- or third-line therapy following failure of immune checkpoint inhibition and platinum doublet chemotherapy administered simultaneously or consecutively.

Since most patients eventually progress following immunochemotherapy and since docetaxel has very limited efficacy in this setting, novel therapies are urgently needed.

### SUMMARY

The present disclosure relates to a combination therapy which comprises an anti-clusterin antibody or an antigen binding fragment thereof and a taxane such as docetaxel.

The present disclosure also relates to a method of treating lung cancer in a subject in need thereof by administering a combination therapy which comprises an anti-clusterin antibody or an antigen binding fragment thereof and a taxane such as docetaxel.

The combination therapy of the present disclosure is used for the treatment of patients having lung cancer (e.g., lung carcinoma) and may more particularly provide a clinical benefit to patients that suffer from non-small cell lung cancer and that have failed prior treatment with an anti-programmed death 1 (anti-PD-1) or anti-programmed death ligand 1 (PD-L1) immune checkpoint antibody and a platinum-containing doublet treatment.

In some embodiments, the combination therapy of the present disclosure is used for treating lung cancer in a subject in need thereof. In particular embodiments, the combination therapy is used to treat non-small cell lung cancer in a subject in need. In another particular embodiments, the combination therapy is used to treat metastatic non-small cell lung cancer in a subject in need.

In some embodiments, a therapeutically effective combination therapy is administered to the subject in need.

Administration of the combination therapy results in a decrease in the size or a stabilization in the size of a target lesion for most patients on treatment. Complete regression of a non-target lesion was also observed in one patient. Surprisingly, the clinical benefit is observable after only two cycles of treatment and in some instances lasts for several weeks.

Moreover, current data suggest an increase in progression-free survival compared to single-agent docetaxel in the same patient population.

The combination therapy of the present application represents an alternative to NSCLC patients that have failed prior treatment that comprises an immune checkpoint antibody. Moreover, evidence of partial response (as defined by Response Evaluation Criteria in Solid Tumors (RECIST) 1.1 criteria) has been observed in four patients having a tumor with a PD-L1 tumor proportion score (TPS) of ≤15% including one patient having a KRAS mutation. Evidence of stable disease as defined by RECIST 1.1 has also been observed in NSCLC patients having a tumor carrying a KRAS mutation.

The combination therapy of the present disclosure may represent an alternative for NSCLC patients that is not eligible for or would unlikely benefit from treatment comprising an immune checkpoint antibody.

Moreover, the combination therapy of the present disclosure may represent an alternative to immune checkpoint antibody monotherapy or combination treatment for NSCLC patients having a tumor with no evidence of PD-L1 expression or with low expression of PD-L1.

Although the combination therapy of the present disclosure comprises docetaxel, another taxane may be selected by a person skilled in the art such as a physician, investigator or the like. As such, exemplary embodiments of taxane includes docetaxel, paclitaxel and derivatives or analogues including for example and without limitations, Abraxane®, Cabazitaxel, larotaxel, milataxel, ortataxel, tesetaxel and others described in Ojima et al., Expert Opin Ther Pat. 2016: 26 (1): 1-20, the entire content of which is incorporated herein by reference.

In some embodiments, the anti-clusterin antibody or antigen binding fragment thereof is selected amongst those that inhibit epithelial to mesenchymal transition in carcinoma cells.

The anti-clusterin antibody or antigen binding fragment thereof of the present disclosure may comprise, for example, an anti-clusterin antibody or antigen binding fragment thereof capable of binding to a C-terminal portion of a β-subunit of human clusterin.

In some embodiments, the anti-clusterin antibody or antigen binding fragment thereof of the present disclosure may comprise, for example, an anti-clusterin antibody or antigen binding fragment thereof capable of binding to amino acids 421 and 443 or to an epitope comprised within amino acids 421 and 443 of a C-terminal portion of a β-subunit of human clusterin.

The anti-clusterin antibody or antigen binding fragment thereof of the present disclosure may comprise, for example, a light chain variable region comprising the complementarity determining regions (CDRs) of the light chain variable region set forth in SEQ ID NO:12 and a heavy chain variable region comprising the CDRs of the heavy chain variable region set forth in SEQ ID NO:13.

The anti-clusterin antibody or antigen binding fragment thereof of the present disclosure may comprise, for example, a light chain variable region comprising CDRs having the amino acid sequence set forth in SEQ ID NO:1 (CDRL1), in SEQ ID NO:2 (CDRL2) and in SEQ ID NO: 3 (CDRL3) and a heavy chain variable region comprising CDRs having the amino acid sequence set forth in SEQ ID NO:4 (CDRH1), SEQ ID NO:5 (CDRH2) and SEQ ID NO: 6 (CDRH3).

The anti-clusterin antibody or antigen binding fragment thereof of the present disclosure may comprise, for example, a light chain variable region comprising CDRs having the amino acid sequence set forth in SEQ ID NO:1 (CDRL1), in SEQ ID NO:2 (CDRL2) and in SEQ ID NO: 3 (CDRL3) and a heavy chain variable region comprising CDRs having the amino acid sequence set forth in SEQ ID NO:7 (CDRH1), SEQ ID NO:8 (CDRH2) and SEQ ID NO: 9 (CDRH3).

In another example, the anti-clusterin antibody or antigen binding fragment thereof of the present disclosure may comprise a light chain variable region having the amino acid sequence set forth in SEQ ID NO: 12 and a heavy chain variable region having the amino acid sequence set forth in SEQ ID NO:13.

In yet another example, the anti-clusterin antibody or antigen binding fragment thereof of the present disclosure may comprise a light chain having the amino acid sequence set forth in SEQ ID NO:14 and a heavy chain having the amino acid sequence set forth in SEQ ID NO:15.

Other exemplary embodiments of anti-clusterin antibodies or antigen binding fragments thereof of the present disclosure include those that may compete with an antibody or antigen binding fragment thereof that comprises the light chain variable region set forth in SEQ ID NO:12 and the heavy chain variable region comprising the CDRs of the heavy chain variable region set forth in SEQ ID NO:13.

In some embodiments, the combination therapy of the present disclosure may be used for treating a subject having lung cancer (e.g., lung carcinoma).

More particularly, the combination therapy of the present disclosure may be used for treating a subject having non-small cell lung cancer.

In some embodiments, the non-small cell lung cancer is metastatic non-small cell lung cancer.

In some embodiments, the metastatic non-small cell lung cancer is stage III non-small cell lung cancer.

In some embodiments, the metastatic non-small cell lung cancer is stage IV non-small cell lung cancer.

In some embodiments, the non-small cell lung cancer is adenocarcinoma.

In some embodiments, the non-small cell lung cancer is squamous cell lung cancer.

In some embodiments, the non-small cell lung cancer is large cell carcinoma.

In some embodiments, the non-small cell lung cancer is adenosquamous carcinoma

In some embodiments, the non-small cell lung cancer (e.g., metastatic non-small cell lung cancer) is characterized has having a KRAS mutation.

Accordingly in one aspect thereof, the combination therapy comprises an anti-clusterin antibody or antigen binding fragment thereof and a taxane for use in the treatment of a subject having non-small cell lung cancer such as for example, metastatic non-small cell lung cancer, wherein the anti-clusterin antibody or antigen binding fragment thereof is as set forth herein.

In some embodiments, the combination therapy of the present disclosure may be used to procure a clinical benefit to a subject having non-small cell lung cancer.

In other embodiments, the combination therapy of the present disclosure may be used to procure a clinical benefit to a subject having metastatic non-small cell lung cancer.

Accordingly, the combination therapy comprises an anti-clusterin antibody or antigen binding fragment thereof and a taxane for use in procuring a clinical benefit to a subject having non-small cell lung cancer such as for example, metastatic non-small cell lung cancer, wherein the anti-clusterin antibody or antigen binding fragment thereof is as set forth herein.

In certain embodiments, the combination therapy is administered in a therapeutically effective dosage.

In certain embodiments, the combination therapy is administered in a safe dosage.

In certain embodiments, the combination therapy is administered at a tolerable dosage.

In certain embodiments, the combination therapy is administered in accordance with a therapeutically effective schedule of administration.

In certain embodiments, the combination therapy is administered at a tolerable schedule of administration.

In certain embodiments, the combination therapy is administered in accordance with a schedule of administration that is safe.

In certain embodiments, the combination therapy is administered in accordance with a therapeutically effective treatment regimen.

In certain embodiments, the combination therapy is administered in accordance with a treatment regimen that is safe.

In certain embodiments, the combination therapy is administered at a tolerable treatment regimen.

In some embodiments, the anti-clusterin antibody or antigen binding fragment thereof is formulated for administration at a dose of between approximately 3 mg/kg to approximately 20 mg/kg.

In some embodiments, the anti-clusterin antibody or antigen binding fragment thereof is formulated as an intravenous infusion.

In some embodiments, the anti-clusterin antibody or antigen binding fragment thereof is formulated as an injectable solution at a concentration of approximately 10 mg/mL.

In some embodiments, the taxane is formulated for administration at a dose of between approximately 50 mg/m² to approximately 100 mg/m², such as for example and without limitations, between approximately 60 mg/m² to 100 mg/m², between approximately 50 mg/m² to 80 mg/m², between approximately 50 mg/m² to 75 mg/m², between approximately 60 mg/m² to 75 mg/m².

In some embodiments, the taxane is docetaxel and is formulated for administration at a dose of between approximately 50 mg/m² to approximately 100 mg/m², such as for example and without limitations, between approximately 60 mg/m² to 100 mg/m², between approximately 50 mg/m² to 80 mg/m², between approximately 50 mg/m² to 75 mg/m², between approximately 60 mg/m² to 75 mg/m².

In some embodiments, docetaxel is formulated as an intravenous infusion.

In some embodiments, docetaxel is formulated is formulated as an injectable solution at a concentration of between approximately 10 mg/mL to approximately 40 mg/mL.

In an exemplary embodiment, the combination therapy comprises an anti-clusterin antibody or antigen binding fragment thereof and docetaxel for use in treating a subject having non-small cell lung cancer such as, for example, metastatic non-small cell lung cancer, where the anti-clusterin antibody or antigen binding fragment thereof is formulated for administration at a dose of approximately 3 mg/kg to approximately 20 mg/kg and comprises a light chain variable region comprising the complementarity determining regions (CDRs) of the light chain variable region set forth in SEQ ID NO:12 and a heavy chain variable region comprising the CDRs of the heavy chain variable region set forth in SEQ ID NO: 13 and docetaxel is formulated for administration at a dose of approximately 50 mg/m² to approximately 100 mg/m².

In another exemplary embodiment, the combination therapy comprises an anti-clusterin antibody or antigen binding fragment thereof and docetaxel for use in treating a subject having non-small cell lung cancer such as, for example, metastatic non-small cell lung cancer, where the anti-clusterin antibody or antigen binding fragment thereof is formulated for administration at a dose of approximately 12 mg/kg and comprises a light chain variable region comprising the complementarity determining regions (CDRs) of the light chain variable region set forth in SEQ ID NO:12 and a heavy chain variable region comprising the CDRs of the heavy chain variable region set forth in SEQ ID NO:13 and docetaxel is formulated for administration at a dose of approximately 50 mg/m² to approximately 75 mg/m².

In yet another exemplary embodiment, the combination therapy comprises an anti-clusterin antibody or antigen binding fragment thereof and docetaxel for use in treating a subject having non-small cell lung cancer such as, for example, metastatic non-small cell lung cancer, where the anti-clusterin antibody or antigen binding fragment thereof is formulated for administration at a dose of approximately 12 mg/kg and comprises a light chain variable region having the amino acid sequence set forth in SEQ ID NO: 12 and a heavy chain variable region having the amino acid sequence set forth in SEQ ID NO:13 and docetaxel is formulated for administration at a dose of approximately 50 mg/m² to approximately 75 mg/m².

In a further exemplary embodiment, the combination therapy comprises an anti-clusterin antibody or antigen binding fragment thereof and docetaxel for use in procuring a clinical benefit to a subject having non-small cell lung cancer such as, for example, metastatic non-small cell lung cancer, where the anti-clusterin antibody or antigen binding fragment thereof is formulated for administration at a dose of approximately 3 mg/kg to approximately 20 mg/kg and comprises a light chain variable region comprising the complementarity determining regions (CDRs) of the light chain variable region set forth in SEQ ID NO:12 and a heavy chain variable region comprising the CDRs of the heavy chain variable region set forth in SEQ ID NO:13 and docetaxel is formulated for administration at a dose of approximately 50 mg/m² to approximately 100 mg/m².

In another exemplary embodiment, the combination therapy comprises an anti-clusterin antibody or antigen binding fragment thereof and docetaxel for use in procuring a clinical benefit to a subject having non-small cell lung cancer such as, for example, metastatic non-small cell lung cancer, where the anti-clusterin antibody or antigen binding fragment thereof is formulated for administration at a dose of approximately 12 mg/kg and comprises a light chain variable region comprising the complementarity determining regions (CDRs) of the light chain variable region set forth in SEQ ID NO:12 and a heavy chain variable region comprising the CDRs of the heavy chain variable region set forth in SEQ ID NO:13 and docetaxel is formulated for administration at a dose of approximately 50 mg/m² to approximately 75 mg/m².

In a further exemplary embodiment, the combination therapy comprises an anti-clusterin antibody or antigen binding fragment thereof and docetaxel for use in procuring a clinical benefit to a subject having non-small cell lung cancer such as, for example, metastatic non-small cell lung cancer, where the anti-clusterin antibody or antigen binding fragment thereof is formulated for administration at a dose of approximately 12 mg/kg and comprises a light chain variable region having the amino acid sequence set forth in SEQ ID NO:12 and a heavy chain variable region having the amino acid sequence set forth in SEQ ID NO:13 and docetaxel is formulated for administration at a dose of approximately 50 mg/m² to approximately 75 mg/m².

In a further exemplary embodiment, the combination therapy is for use in procuring a clinical benefit to a subject having non-small cell lung cancer, wherein the combination therapy is used for at least two cycles of treatment each cycle of treatment comprising administration of the anti-clusterin antibody at a dose of approximately 3 mg/kg to approximately 20 mg/kg once per week, once every two weeks, once every three weeks, once every four weeks and administration of a taxane at a dose of approximately 50 mg/m² to 100 mg/m² once every two to three weeks, wherein the anti-clusterin antibody comprises a light chain variable region comprising the complementarity determining regions (CDRs) of the light chain variable region set forth in SEQ ID NO:12 and a heavy chain variable region comprising the CDRs of the heavy chain variable region set forth in SEQ ID NO:13.

In yet a further exemplary embodiment, the combination therapy is for use in procuring a clinical benefit to a subject having non-small cell lung cancer, wherein the combination therapy is used for at least two cycles of treatment each cycle of treatment comprising administration of the anti-clusterin antibody at a dose of approximately 12 mg/kg once per week, once every two weeks, once every three weeks, once every four weeks and administration of docetaxel at a dose of approximately 75 mg/m² once every three weeks, wherein the anti-clusterin antibody comprises a light chain variable region comprising the complementarity determining regions (CDRs) of the light chain variable region set forth in SEQ ID NO:12 and a heavy chain variable region comprising the CDRs of the heavy chain variable region set forth in SEQ ID NO: 13.

In yet a further exemplary embodiment, the combination therapy is for use in procuring a clinical benefit to a subject having non-small cell lung cancer, wherein the combination therapy is used for at least two cycles of treatment each cycle of treatment comprising administration of the anti-clusterin antibody at a dose of approximately 12 mg/kg once per week, once every two weeks, once every three weeks, once every four weeks and administration of docetaxel at a dose of approximately 50 mg/m² once every two weeks, wherein the anti-clusterin antibody comprises a light chain variable region comprising the complementarity determining regions (CDRs) of the light chain variable region set forth in SEQ ID NO:12 and a heavy chain variable region comprising the CDRs of the heavy chain variable region set forth in SEQ ID NO:13.

In some embodiments, the clinical benefit is defined as per RECIST 1.1 guidelines.

In some embodiments, the combination therapy is used to procure a clinical benefit for at least 6 weeks. In certain embodiments, the combination therapy is used to procure a clinical benefit for at least 6 weeks after first determination of response to treatment.

In some embodiments, the combination therapy is used to procure a clinical benefit for at least 12 weeks. In certain embodiments, the combination therapy is used to procure a clinical benefit for at least 12 weeks after first determination of response to treatment.

In some embodiments, the combination therapy is used to procure a clinical benefit for at least 18 weeks. In certain embodiments, the combination therapy is used to procure a clinical benefit for at least 18 weeks after first determination of response to treatment.

In some embodiments, the combination therapy is used to procure a clinical benefit for at least 24 weeks. In certain embodiments, the combination therapy is used to procure a clinical benefit for at least 24 weeks after first determination of response to treatment.

In some embodiments, the combination therapy is used to procure a clinical benefit for at least 30 weeks. In certain embodiments, the combination therapy is used to procure a clinical benefit for at least 30 weeks after first determination of response to treatment. In some embodiments, the combination therapy is used to procure a clinical benefit for at least 36 weeks. In certain embodiments, the combination therapy is used to procure a clinical benefit for at least 36 weeks after first determination of response to treatment. In certain embodiments, the combination therapy is used to procure a clinical benefit for at least 42 weeks after first determination of response to treatment. In certain embodiments, the combination therapy is used to procure a clinical benefit for at least 42 weeks after first determination of response to treatment.

In some embodiments, the combination therapy is used to procure a clinical benefit for at least 48 weeks. In certain embodiments, the combination therapy is used to procure a clinical benefit for at least 48 weeks after first determination of response to treatment.

In some embodiments, the combination therapy is used to procure a clinical benefit for at least 54 weeks. In certain embodiments, the combination therapy is used to procure a clinical benefit for at least 54 weeks after first determination of response to treatment.

In some embodiments, the combination therapy is used to procure a clinical benefit for at least 60 weeks. In certain embodiments, the combination therapy is used to procure a clinical benefit for at least 60 weeks after first determination of response to treatment.

In some embodiments, the combination therapy is used to procure a clinical benefit for at least 66 weeks. In certain embodiments, the combination therapy is used to procure a clinical benefit for at least 66 weeks after first determination of response to treatment.

In accordance with the present disclosure, the subject in need is a subject as described herein.

The present disclosure therefore involves methods involving administration of the combination therapy to a subject in need.

In accordance with the present disclosure, the anti-clusterin antibody or antigen binding fragment thereof is administered once per week.

In accordance with the present disclosure, the anti-clusterin antibody or antigen binding fragment thereof is administered once every two weeks.

In accordance with the present disclosure, the anti-clusterin antibody or antigen binding fragment thereof is administered once every three weeks.

In accordance with the present disclosure, the anti-clusterin antibody or antigen binding fragment thereof is administered once every four weeks.

In accordance with the present disclosure, docetaxel is administered once per week. In accordance with the present disclosure, docetaxel is administered once every two weeks.

In accordance with the present disclosure, docetaxel is administered once every three weeks.

In accordance with the present disclosure, docetaxel is administered once every four weeks.

It is to be understood herein that the frequency of treatment (i.e., once per week, every two weeks, every three weeks, every four weeks and the like) is not strict and allow some variation. For example, a physician may decide to administer a dose ahead of the schedule or delay the dose by a few days or longer. As well, it is also possible that one or more doses be skipped without affecting treatment.

In some embodiments, the anti-clusterin antibody or antigen binding fragment thereof is administered at a dose of between approximately 3 mg/kg to approximately 20 mg/kg.

In some embodiments, the taxane is administered at a dose of between approximately 50 mg/m² to approximately 100 mg/m², such as for example and without limitations, between approximately 60 mg/m² to approximately 100 mg/m², between approximately 50 mg/m² to approximately 80 mg/m², between approximately 50 mg/m² to approximately 75 mg/m², between approximately 60 mg/m² to approximately 75 mg/m².

In some embodiments, the taxane is docetaxel and is administered at a dose of between approximately 50 mg/m² to approximately 100 mg/m², such as for example and without limitations, between approximately 60 mg/m² to approximately 100 mg/m², between approximately 50 mg/m² to approximately 80 mg/m², between approximately 50 mg/m² to approximately 75 mg/m², between approximately 60 mg/m² to approximately 75 mg/m².

In some embodiments, the taxane is docetaxel and is administered at a dose of between 50 mg/m² to 100 mg/m², such as for example and without limitations, between 60 mg/m² to 100 mg/m², between 50 mg/m² to 80 mg/m², between 50 mg/m² to 75 mg/m², between 60 mg/m² to 75 mg/m².

In exemplary embodiments, the anti-clusterin antibody or antigen binding fragment thereof is administered at a dose of 12 mg/kg once weekly (once per week), once every two weeks, or once every three weeks and docetaxel is administered at a dose of 75 mg/m² once every three weeks.

In exemplary embodiments the anti-clusterin antibody or antigen binding fragment thereof is administered at a dose of 12 mg/kg once weekly (once per week), once every two weeks, or once every three weeks and docetaxel is administered at a dose of 60 mg/m² once every three weeks.

In exemplary embodiments the anti-clusterin antibody or antigen binding fragment thereof is administered at a dose of 9 mg/kg once weekly (once per week), once every two weeks, or once every three weeks and docetaxel is administered at a dose of 75 mg/m² once every three weeks.

In exemplary embodiments the anti-clusterin antibody or antigen binding fragment thereof is administered at a dose of 9 mg/kg once weekly (once per week), once every two weeks, or once every three weeks and docetaxel is administered at a dose of 60 mg/m² once every three weeks.

In exemplary embodiments the anti-clusterin antibody or antigen binding fragment thereof is administered at a dose of 6 mg/kg once weekly (once per week), once every two weeks, or once every three weeks and docetaxel is administered at a dose of 75 mg/m² once every three weeks.

In exemplary embodiments the anti-clusterin antibody or antigen binding fragment thereof is administered at a dose of 6 mg/kg once weekly (once per week), once every two weeks, or once every three weeks and docetaxel is administered at a dose of 60 mg/m² once every three weeks.

In exemplary embodiments the anti-clusterin antibody or antigen binding fragment thereof is administered at a dose of 3 mg/kg once weekly (once per week), once every two weeks, or once every three weeks and docetaxel is administered at a dose of 75 mg/m² once every three weeks.

In exemplary embodiments the anti-clusterin antibody or antigen binding fragment thereof is administered at a dose of 3 mg/kg once weekly (once per week), once every two weeks, or once every three weeks and docetaxel is administered at a dose of 60 mg/m² once every three weeks.

In exemplary embodiments, the anti-clusterin antibody or antigen binding fragment thereof is administered at a dose of 12 mg/kg once weekly (once per week), once every two weeks, or once every three weeks and docetaxel is administered at a dose of 50 mg/m² once every three weeks.

In exemplary embodiments the anti-clusterin antibody or antigen binding fragment thereof is administered at a dose of 9 mg/kg once weekly (once per week), once every two weeks, or once every three weeks and docetaxel is administered at a dose of 50 mg/m² once every three weeks.

In exemplary embodiments the anti-clusterin antibody or antigen binding fragment thereof is administered at a dose of 6 mg/kg once weekly (once per week), once every two weeks, or once every three weeks and docetaxel is administered at a dose of 50 mg/m² once every three weeks.

In exemplary embodiments the anti-clusterin antibody or antigen binding fragment thereof is administered at a dose of 3 mg/kg once weekly (once per week), once every two weeks, or once every three weeks and docetaxel is administered at a dose of 50 mg/m² once every three weeks.

In exemplary embodiments, the anti-clusterin antibody or antigen binding fragment thereof is administered at a dose of 12 mg/kg once weekly (once per week), once every two weeks, or once every three weeks and docetaxel is administered at a dose of 50 mg/m² once every two weeks.

In exemplary embodiments the anti-clusterin antibody or antigen binding fragment thereof is administered at a dose of 9 mg/kg once weekly (once per week), once every two weeks, or once every three weeks and docetaxel is administered at a dose of 50 mg/m² once every two weeks.

In exemplary embodiments the anti-clusterin antibody or antigen binding fragment thereof is administered at a dose of 6 mg/kg once weekly (once per week), once every two weeks, or once every three weeks and docetaxel is administered at a dose of 50 mg/m² once every two weeks.

In exemplary embodiments the anti-clusterin antibody or antigen binding fragment thereof is administered at a dose of 3 mg/kg once weekly (once per week), once every two weeks, or once every three weeks and docetaxel is administered at a dose of 50 mg/m² once every two weeks.

In some embodiments, the anti-clusterin antibody or antigen binding fragment thereof and docetaxel may be administered on same day.

In some embodiments, the anti-clusterin antibody or antigen binding fragment thereof and docetaxel may be administered on same day and separately.

In some embodiments, the anti-clusterin antibody or antigen binding fragment thereof and/or docetaxel may be administered by infusion.

In some embodiments, the anti-clusterin antibody or antigen binding fragment thereof and docetaxel are both administered over the entire course of the treatment period.

In some embodiments, the anti-clusterin antibody or antigen binding fragment thereof and docetaxel are both administered at each cycle of treatment.

The present disclosure therefore relates to a method of treating a subject having non-small cell lung cancer with the combination therapy disclosed herein.

The present disclosure also relates to a method of treating a subject having metastatic non-small cell lung cancer with the combination therapy disclosed herein.

Accordingly, in certain aspects, the present disclosure provides a method of treating a subject having non-small cell lung cancer such as, for example, metastatic non-small cell lung cancer comprising administering a combination therapy comprising an anti-clusterin antibody or antigen binding fragment thereof and docetaxel, where the anti-clusterin antibody or antigen binding fragment thereof is administered at a dose of approximately 3 mg/kg to approximately 20 mg/kg and comprises a light chain variable region comprising the complementarity determining regions (CDRs) of the light chain variable region set forth in SEQ ID NO:12 and a heavy chain variable region comprising the CDRs of the heavy chain variable region set forth in SEQ ID NO:13 and docetaxel is administered at a dose of approximately 50 mg/m² to approximately 100 mg/m².

In an exemplary embodiment, the method is a method of treating a subject having non-small cell lung cancer such as, for example, metastatic non-small cell lung cancer comprising administering a combination therapy comprising an anti-clusterin antibody or antigen binding fragment thereof and docetaxel, where the anti-clusterin antibody or antigen binding fragment thereof is administered at a dose of approximately 12 mg/kg and comprises a light chain variable region comprising the complementarity determining regions (CDRs) of the light chain variable region set forth in SEQ ID NO: 12 and a heavy chain variable region comprising the CDRs of the heavy chain variable region set forth in SEQ ID NO:13 and docetaxel is administered at a dose of approximately 50 mg/m² to approximately 75 mg/m².

In an exemplary embodiment, the method is a method of treating a subject having non-small cell lung cancer such as, for example, metastatic non-small cell lung cancer comprising administering a combination therapy comprising an anti-clusterin antibody or antigen binding fragment thereof and docetaxel, where the anti-clusterin antibody or antigen binding fragment thereof is administered at a dose of approximately 12 mg/kg and comprises a light chain variable region comprising the amino acid sequence set forth in SEQ ID NO:12 and a heavy chain variable region comprising the amino acid sequence set forth in SEQ ID NO: 13 and docetaxel is administered at a dose of approximately 50 mg/m² to approximately 75 mg/m².

In a particular embodiment, the present disclosure relates to a method of treating non-small cell lung cancer (NSCLC) in a subject in need thereof, comprising administering a combination therapy for at least one cycle of treatment comprising administration of the anti-clusterin antibody at a dose of approximately 12 mg/kg once per week, once every two weeks, once every three weeks or once every four weeks and administration of docetaxel at a dose of between approximately 50 mg/m² to approximately 75 mg/m² once every two to three weeks, wherein the anti-clusterin antibody comprises a light chain variable region comprising the complementarity determining regions (CDRs) of the light chain variable region set forth in SEQ ID NO: 12 and a heavy chain variable region comprising the CDRs of the heavy chain variable region set forth in SEQ ID NO:13.

The present disclosure also relates to a method of treating a subject having non-small cell lung cancer with the combination therapy disclosed herein so as to obtain and/or maintain a clinical benefit.

The present disclosure further relates to a method of treating a subject having metastatic non-small cell lung cancer with the combination therapy disclosed herein so as to obtain and/or maintain a clinical benefit.

Accordingly, in certain aspects, the present disclosure provides a method of treating a subject having non-small cell lung cancer such as, for example, metastatic non-small cell lung cancer comprising administering a combination therapy comprising an anti-clusterin antibody or antigen binding fragment thereof and docetaxel so as to obtain a clinical benefit, where the anti-clusterin antibody or antigen binding fragment thereof is administered at a dose of approximately 3 mg/kg to approximately 20 mg/kg and comprises a light chain variable region comprising the complementarity determining regions (CDRs) of the light chain variable region set forth in SEQ ID NO:12 and a heavy chain variable region comprising the CDRs of the heavy chain variable region set forth in SEQ ID NO:13 and docetaxel is administered at a dose of approximately 50 mg/m² to approximately 100 mg/m².

In an exemplary embodiment, the method is a method of treating a subject having non-small cell lung cancer such as, for example, metastatic non-small cell lung cancer comprising administering a combination therapy comprising an anti-clusterin antibody or antigen binding fragment thereof and docetaxel so as to obtain a clinical benefit, where the anti-clusterin antibody or antigen binding fragment thereof is administered at a dose of approximately 12 mg/kg and comprises a light chain variable region comprising the complementarity determining regions (CDRs) of the light chain variable region set forth in SEQ ID NO:12 and a heavy chain variable region comprising the CDRs of the heavy chain variable region set forth in SEQ ID NO:13 and docetaxel is administered at a dose of approximately 50 mg/m² to approximately 75 mg/m².

In an exemplary embodiment, the method is a method of treating a subject having non-small cell lung cancer such as, for example, metastatic non-small cell lung cancer comprising administering a combination therapy comprising an anti-clusterin antibody or antigen binding fragment thereof and docetaxel so as to obtain a clinical benefit, where the anti-clusterin antibody or antigen binding fragment thereof is administered at a dose of approximately 12 mg/kg and comprises a light chain variable region comprising the amino acid sequence set forth in SEQ ID NO:12 and a heavy chain variable region comprising the amino acid sequence set forth in SEQ ID NO:13 and docetaxel is administered at a dose of approximately 50 mg/m² to approximately 75 mg/m².

The present disclosure also relates to a method of procuring a clinical benefit to a subject having non-small cell lung cancer with the combination therapy disclosed herein.

The present disclosure further relates to a method of procuring a clinical benefit to a subject having metastatic non-small cell lung cancer with the combination therapy disclosed herein.

Exemplary embodiments of clinical benefit include, without limitation reduction in the size of a lesion, stabilization in the size of a lesion, regression of a lesion, complete response (CR) as per RECIST 1.1 guidelines, partial response (PR) as per RECIST 1.1 guidelines, stable disease (SD) as per RECIST 1.1 guidelines, improvement in a symptom associated with the cancer, and/or reduction in or an elimination of metabolic activity in a lesion etc.

In some instances, the reduction or stabilization in size or the regression may be observed in a target lesion.

In some instances, the reduction or stabilization in size or the regression may be observed in a non-target lesion.

Accordingly, in certain aspects, the present disclosure provides a method of procuring a clinical benefit to a subject having non-small cell lung cancer such as, for example, metastatic non-small cell lung cancer comprising administering a combination therapy comprising an anti-clusterin antibody or antigen binding fragment thereof and docetaxel, where the anti-clusterin antibody or antigen binding fragment thereof is administered at a dose of approximately 3 mg/kg to approximately 20 mg/kg and comprises a light chain variable region comprising the complementarity determining regions (CDRs) of the light chain variable region set forth in SEQ ID NO:12 and a heavy chain variable region comprising the CDRs of the heavy chain variable region set forth in SEQ ID NO:13 and docetaxel is administered at a dose of approximately 50 mg/m² to approximately 100 mg/m².

In an exemplary embodiment, the method is a method of procuring a clinical benefit to a subject having non-small cell lung cancer such as, for example, metastatic non-small cell lung cancer comprising administering a combination therapy comprising an anti-clusterin antibody or antigen binding fragment thereof and docetaxel, where the anti-clusterin antibody or antigen binding fragment thereof is administered at a dose of approximately 12 mg/kg and comprises a light chain variable region comprising the complementarity determining regions (CDRs) of the light chain variable region set forth in SEQ ID NO:12 and a heavy chain variable region comprising the CDRs of the heavy chain variable region set forth in SEQ ID NO: 13 and docetaxel is administered at a dose of approximately 50 mg/m² to approximately 75 mg/m².

In an exemplary embodiment, the method is a method of procuring a clinical benefit to a subject having non-small cell lung cancer such as, for example, metastatic non-small cell lung cancer comprising administering a combination therapy comprising an anti-clusterin antibody or antigen binding fragment thereof and docetaxel, where the anti-clusterin antibody or antigen binding fragment thereof is administered at a dose of approximately 12 mg/kg and comprises a light chain variable region comprising the amino acid sequence set forth in SEQ ID NO: 12 and a heavy chain variable region comprising the amino acid sequence set forth in

SEQ ID NO:13 and docetaxel is administered at a dose of approximately 50 mg/m² to approximately 75 mg/m².

In accordance with the present disclosure, the combination therapy is administered in dosages sufficient to result in a clinical benefit.

In accordance with the present disclosure, the combination therapy is administered for a period of time sufficient to result in a clinical benefit.

In accordance with the present disclosure, the combination therapy is administered according to a schedule of treatment sufficient to result in a clinical benefit.

In accordance with the present disclosure, the combination therapy is administered according to treatment regimen sufficient to result in a clinical benefit.

In an exemplary embodiment, the combination therapy is administered in one or more cycles of treatment including administration of the anti-clusterin antibody or antigen binding fragment thereof once per week, once every two weeks, or once every three weeks and administration of docetaxel once every three weeks. It is to be understood that the interval of treatment may be changed without significantly changing the effect of the combined therapy.

In certain embodiments, the combination therapy is administered for at least two cycles of treatment.

In certain embodiments, the combination therapy is administered for more than two cycles of treatment.

The present disclosure also provides a method of procuring a clinical benefit to a subject having non-small cell lung cancer, in a method of treatment which comprises administering a combination therapy for at least two cycles of treatment, each cycle of treatment comprising administration of the anti-clusterin antibody at a dose of approximately 3 mg/kg to approximately 20 mg/kg once per week, once every two weeks, or once every three weeks and administration of a taxane at a dose of approximately 50 mg/m² to 100 mg/m² once every two to three weeks, wherein the anti-clusterin antibody comprises a light chain variable region comprising the complementarity determining regions (CDRs) of the light chain variable region set forth in SEQ ID NO:12 and a heavy chain variable region comprising the CDRs of the heavy chain variable region set forth in SEQ ID NO:13.

Accordingly, in other aspects, the present disclosure provides a method of procuring a clinical benefit to a subject having non-small cell lung cancer, in a method of treatment which comprises administering a combination therapy for at least two cycles of treatment each cycle of treatment comprising administration of the anti-clusterin antibody at a dose of approximately 12 mg/kg once per week, once every two weeks, or once every three weeks and administration of docetaxel at a dose of approximately 75 mg/m² once every three weeks, wherein the anti-clusterin antibody comprises a light chain variable region comprising the complementarity determining regions (CDRs) of the light chain variable region set forth in SEQ ID NO:12 and a heavy chain variable region comprising the CDRs of the heavy chain variable region set forth in SEQ ID NO:13.

Accordingly, in other aspects, the present disclosure provides a method of procuring a clinical benefit to a subject having non-small cell lung cancer, in a method of treatment which comprises administering a combination therapy for at least two cycles of treatment each cycle of treatment comprising administration of the anti-clusterin antibody at a dose of approximately 12 mg/kg once per week, once every two weeks, or once every three weeks and administration of docetaxel at a dose of approximately 50 mg/m² once every two weeks, wherein the anti-clusterin antibody comprises a light chain variable region comprising the complementarity determining regions (CDRs) of the light chain variable region set forth in SEQ ID NO:12 and a heavy chain variable region comprising the CDRs of the heavy chain variable region set forth in SEQ ID NO: 13.

In some embodiments, the clinical benefit is regression of a target lesion. In some embodiments, the clinical benefit is a decrease in the size of a target lesion. In some embodiments, the clinical benefit is a stabilization in the size of a target lesion.

In some embodiments, the clinical benefit is a reduction in the number of tumor cells in a target lesion.

In other embodiments, the clinical benefit is regression of a non-target lesion. In yet other embodiments, the clinical benefit is a decrease in the size of a non-target lesion.

In further embodiments, the clinical benefit is a stabilization in the size of a non-target lesion.

In yet further embodiments, the clinical benefit is a reduction in the number of tumor cells in a non-target lesion.

In some embodiments, the clinical benefit is defined as per RECIST 1.1 guidelines.

In some embodiments, the clinical benefit may be qualitative, such as for example, an improvement in the quality of life (e.g., health-related quality of life).

It is to be understood herein that the clinical benefit may last for a certain period of time. Duration of clinical benefit may vary from subjects to subjects.

In accordance with the present disclosure, the combination therapy may reduce tumor burden.

It is to be understood herein that tumor burden may be assessed by measuring the size of one or more tumor lesions. In other embodiments, tumor burden may be determined by assessing tumor markers in solid biopsies, liquid biopsies, blood sample, serum sample, urine sample and the like. For example, tumor burden is assessed by detecting circulating tumor cells (CTCs). In another example, tumor burden is assessed by detecting circulating tumor DNA.

In certain embodiments the method procures a clinical benefit for at least 6 weeks after first determination of response to treatment.

In certain embodiments the method procures a clinical benefit for at least 12 weeks after first determination of response to treatment.

In certain embodiments the method procures a clinical benefit for at least 18 weeks after first determination of response to treatment.

In certain embodiments the method procures a clinical benefit for at least 24 weeks after first determination of response to treatment.

In certain embodiments the method procures a clinical benefit for at least 30 weeks after first determination of response to treatment.

In certain embodiments the method procures a clinical benefit for at least 36 weeks after first determination of response to treatment.

In certain embodiments the method procures a clinical benefit for at least 42 weeks after first determination of response to treatment.

In certain embodiments the method procures a clinical benefit for at least 48 weeks after first determination of response to treatment.

In certain embodiments the method procures a clinical benefit for at least 54 weeks after first determination of response to treatment.

In certain embodiments the method procures a clinical benefit for at least 60 weeks after first determination of response to treatment.

In certain embodiments the method procures a clinical benefit for at least 66 weeks after first determination of response to treatment.

Once a physician prescribes the combination treatment, the subject's health and condition is monitored. The monitoring can include a physical examination, an interview with the subject concerning its quality of life, obtaining biological samples and/or performing a scan to monitor progression of the lung cancer and/or the presence of metastasis. Treatment is continued so long as the subject experiences a clinical benefit (qualitative or quantitative). Treatment may be discontinued once tumor progression is observed.

In some embodiments, the anti-clusterin antibody or antigen binding fragment thereof is used as an IV infusion.

In some embodiments, docetaxel is used as an IV infusion.

In certain embodiments, the combination therapy may promote infiltration of immune cells in the tumor microenvironment of one or more lesions (target and/or non-target lesions).

In certain embodiments, the combination therapy may promote infiltration of lymphocytes in the tumor microenvironment of one or more lesions (target and/or non-target lesions).

In certain embodiments, the combination therapy may promote infiltration of T-cells in the tumor microenvironment of one or more lesions (target and/or non-target lesions).

In accordance with the present disclosure, the method may result in the tumor or lesions being more susceptible to treatment by immunotherapy.

The method of the present disclosure may therefore also include a step of administering immunotherapy subsequently to the combination therapy disclosed herein.

In some embodiments, the immunotherapy comprises cellular immunotherapy (CAR-T, TILs, etc.).

In some embodiments, the immunotherapy comprises an immune checkpoint inhibitor.

Accordingly, the present disclosure relates to a method of treating NSCLC such as metastatic NSCLC by promoting infiltration of immune cells in one or more lesions.

In an embodiment, the present disclosure encompasses a method of treating NSCLC such as metastatic NSCLC by promoting infiltration of immune cells in one or more lesions that have low expression of PD-L1 or no evidence of PD-L1 expression.

In accordance with the present disclosure, a lesion may be characterized as having low expression of PD-L1 when the PD-L1 tumor proportion score (TPS) of such lesion is, for example, ≤15%.

More particularly, a lesion may be characterized as having low expression of PD-L1 when the PD-L1 TPS of such lesion is, for example, <5%.

More specifically, a lesion may be characterized as having low expression of PD-L1 when the PD-L1 TPS of such lesion is, for example, ≤1%.

Even more specifically, a lesion may be characterized as having low expression of PD-L1 when the PD-L1 TPS of such lesion is, for example, <1%.

In accordance with the present disclosure, a lesion is characterized as having no evidence of PD-L1 expression when the PD-L1 TPS of such lesion of is 0% or when PD-L1 expression undetectable.

In certain embodiments, the combination therapy may promote reduction in the number of tumor cells in one or more lesions.

In an exemplary embodiment, the combination therapy may result in a decrease in the size of a lesion.

It is to be understood herein that a lesion is considered to decrease in size when measurements indicates that it is smaller than previous measurements or than baseline measurement.

In some exemplary embodiment, administration of the combination therapy may result in a decrease of at least 5%, at least 10%, at least 15%, at least 20%, at least 25%, at least 30%, at least 35%, at least 40%, at least 45%, at least 50% in the size of a lesion.

In another exemplary embodiment, the combination therapy may result in a stabilization in the size of a lesion.

In yet another exemplary embodiment, the combination therapy may result in an increase of less than 20% in the size of a lesion. It is also to be understood herein that an increase of less than approximately 20% in the size of a lesion, in comparison with previous measurements or baseline measurements is considered as a stabilization in the growth of a lesion.

In yet another exemplary embodiment, the combination therapy may result in regression of a lesion. In some instances, the regression may be complete. In other instances, the regression may be partial.

In some embodiments, the combination therapy may result in a stabilization in the size of a lesion.

In some instances, the size of a lesion may increase or may remain stable while the number of tumor cells within the lesion decreases. For example, tumor cells might be replaced by fibrotic tissue and inflammatory cells and as such no decrease in the size of a lesion is detected. Accordingly, in some instances, the number of live tumor cells may decrease, and the number of immune cells may increase thereby resulting in pseudogrogression.

As such, in another exemplary embodiment, the combination therapy may result in a reduction in the number of tumor cells in a lesion.

It is to be understood herein that the size of a lesion may be assessed by several methods including for example and without limitations, by CT scan.

In a further exemplary embodiment, the combination therapy may result in a reduction in or an elimination of metabolic activity in a lesion. It is to be understood herein that the metabolic activity of a lesion may be assessed by several methods including for example and without limitation, by positron emission tomography (PET) scan.

It is also to be understood herein that an increase in the size of a lesion conjugated with a decrease in the number of tumor cells within that lesion is also considered as a stabilization in the growth of a lesion.

It is also to be understood herein that a reduction of the metabolic activity of a lesion (with or without growth) is also considered as a stabilization in the growth of a lesion.

In yet a further exemplary embodiment, the combination therapy may result in an improvement in a symptom associated with the lung cancer.

In another embodiment, administration of the combination therapy may result in a complete response (CR) as per RECIST 1.1 guidelines.

In additional embodiments, administration of the combination therapy may result in partial response (PR) as per RECIST 1.1 guidelines.

In yet another embodiment, administration of the combination therapy may result in a stable disease (SD) as per RECIST 1.1 guidelines.

In accordance with the present disclosure, the combination therapy is used or for use as long as a clinical benefit is observed.

In accordance with the present disclosure, the combination therapy is used or for use until tumor progression is observed.

In accordance with the present disclosure, the combination therapy is used or for use until unmanageable toxicity occurs.

In some embodiments, administration of the anti-clusterin antibody or antigen binding fragment thereof may be maintained after cessation of the combination therapy.

In some embodiments, administration of the anti-clusterin antibody or antigen binding fragment thereof may be maintained after the appearance of clinical benefits.

It is to be understood herein that a clinical benefit may be evidenced by assessing tumor biomarkers.

In accordance with the present disclosure, the combination therapy is for use in a subject as described herein.

In accordance with the present disclosure, the subject is a human subject. In accordance with the present disclosure, the subject is an adult (i.e., ≥18 years of age).

In some embodiments, the subject has or is selected for having NSCLC.

In some embodiments, the subject has or is selected for having metastatic NSCLC.

In some embodiments, the subject has or is selected for having NSCLC, such as metastatic NSCLC, that is not eligible for or would unlikely benefit from treatment comprising an immune checkpoint antibody.

In some embodiments, the subject has or is selected for having NSCLC, such as metastatic NSCLC, that progressed after treatment comprising an immune checkpoint antibody.

In some embodiments, the subject has or is selected for having NSCLC such as metastatic NSCLC that progressed after chemotherapy.

In other embodiments, the subject has or is selected for having NSCLC, such as metastatic NSCLC, that has failed prior treatment with immune checkpoint antibody and chemotherapy combination treatment.

In other embodiments, the subject has or is selected for having NSCLC, such as metastatic NSCLC, that has failed prior treatment with immune checkpoint antibody and a platinum-containing doublet treatment.

In additional embodiments, the subject has or is selected for having NSCLC, such as metastatic NSCLC, that has failed prior treatment with an anti-PD-1 or PD-L1 immune checkpoint antibody and a platinum-containing doublet treatment.

In accordance with the present disclosure, the doublet treatment may be provided simultaneously.

In accordance with the present disclosure, the doublet treatment may be provided sequentially.

In some embodiments, the subject has or is selected for having NSCLC, such as metastatic NSCLC, that has failed prior treatment with an anti-PD-1 immune checkpoint antibody.

In some embodiments, the subject has or is selected for having NSCLC, such as metastatic NSCLC, that has failed prior treatment with an PD-L1 immune checkpoint antibody.

In some embodiments, the subject has or is selected for having NSCLC, such as metastatic NSCLC, that has failed prior treatment with an anti-PD-1 or PD-L1 immune checkpoint antibody.

In some embodiments, the subject has or is selected for having NSCLC, such as metastatic NSCLC, that has failed prior treatment with an anti-PD-1 immune checkpoint antibody and a platinum-containing doublet treatment.

In some embodiments, the subject has or is selected for having NSCLC, such as metastatic NSCLC, that has failed prior treatment with an anti-PD-L1 immune checkpoint antibody and a platinum-containing doublet treatment.

In some embodiments, the subject has or is selected for having NSCLC, such as metastatic NSCLC, that has failed prior treatment with an anti-PD-1 or PD-L1 immune checkpoint antibody and a platinum-containing doublet treatment.

In some embodiments, the subject has or is selected for having NSCLC, such as metastatic NSCLC, that has failed prior treatment with immune checkpoint antibody selected from ipilimumab, nivolumab, pembrolizumab, cemiplimab, atezolizumab, avelumab, or durvalumab alone or in combination with platinum-based chemotherapy.

In some embodiments, the subject has or is selected for having NSCLC, such as metastatic NSCLC, that has failed prior treatment with pembrolizumab alone or in combination with platinum-based chemotherapy.

In some embodiments, the subject has or is selected for having a PD-L1 tumor proportion score (TPS) of ≥50%.

In some embodiments, the subject has or is selected for having a PD-L1 tumor proportion score (TPS) of <50%.

In some embodiments, the subject has or is selected for having a PD-L1 tumor proportion score (TPS) of between 1 to 49%.

In some embodiments, the subject has or is selected for having a PD-L1 tumor proportion score of <1%.

In some embodiments, the subject has or is selected for having a tumor that has poor infiltration of immune cells. The level of immune cell infiltration may be determined by a trained pathologist or scientist. The level of immune cell infiltration may be determined by imaging using computer-based quantification of tumor biopsy.

In some embodiments, the subject has or is selected for having one or more lesions having an EMT signature or showing signs of an EMT signature. The EMT signature of the tumor may be determined by a trained pathologist or scientist. The EMT signature of the tumor may be determined by imaging using computer-based quantification of tumor or tumor biopsy.

In some embodiments, the EMT signature may be assessed by one or more EMT biomarkers. For example, an EMT signature may be associated with the acquisition of mesenchymal markers and/or with attenuation of epithelial markers especially associated with EMT type 3 (see Zeisber, M. and E. G. Neilson, The Journal of Clin Investig, 119:1429-1437 (2009)).

EMT biomarkers include for example, cell-surface proteins such as, for example, N-cadherin, OB-cadherin, α5β1 integrin, αVβ6 integrin, syndecan-1, cytoskeletal markers such as, for example, FSP1, α-SMA, vimentin, β-catenin, α1(I) collagen, α1(III) collagen, fibronectin, laminin 5, transcription factors such as, for example, snail1, snail2, ZEB1, CBF-A/KAP-1 complex, twist, LEF-1, Ets-1. FOXC2, goosecoid, and mircroRNAs such as, for example, miR10b, miR-21 (acquired markers), E-cadherin, ZO-1, cytokeratin, «1(IV) collagen, laminin-1 (attenuated markers) (see Zeisber, M. and E. G. Neilson, The Journal of Clin Investig, 119:1429-1437 (2009)).

In accordance with the present disclosure, the subject in need has a tumor that expresses or secretes clusterin.

In some embodiments, the subject has or is selected for having stage III NSCLC.

In some embodiments, the subject has or is selected for having stage IV NSCLC.

In some embodiments, the subject does not receive concurrent anti-cancer treatment other than the anti-clusterin antibody or antigen binding fragment thereof and/or docetaxel.

In some embodiments, the subject has or is selected for not being immunosuppressed.

In accordance with the present disclosure, the subject has or is selected for not having received an immunosuppressive medication within 14, 13, 12, 11, 10, 9, 8, 7, 6, 5, 4, 3, 2 days or 1 day prior to treatment.

In some embodiments, the subject has or is selected for not having received an immunosuppressive medication within 7 days prior to treatment.

In accordance with the present disclosure, the subject has or is selected for not having received prior treatment with docetaxel.

The method of the present disclosure comprises administering one or more cycles of treatment to a subject in need.

In some embodiments, the method comprises administering to the subject in need, at least one initial cycle of treatment and at least one subsequent cycle of treatment. The initial cycle(s) of treatment and subsequent cycle(s) of treatment may be the same or different. For example, the initial cycle(s) of treatment may include a given dose of the anti-clusterin antibody or antigen binding fragment thereof, while the subsequent cycle(s) of treatment may include a different dose of the anti-clusterin antibody or antigen binding fragment thereof. In another example, the initial cycle(s) of treatment may include a given dose of docetaxel, while the subsequent cycle(s) of treatment may include a different dose of docetaxel. In yet another example, the time interval between the anti-clusterin antibody or antigen binding fragment thereof and docetaxel may be different in the initial cycle(s) of treatment and in the subsequent cycle(s) of treatment. In some embodiments, the initial cycle of treatment comprises at least one dose of an anti-clusterin antibody or antigen binding fragment thereof and at least one dose of taxane such as docetaxel. In exemplary embodiments, the initial cycle of treatment comprises two doses of an anti-clusterin antibody or antigen binding fragment thereof and one dose of taxane such as docetaxel. In exemplary embodiments, the initial cycle of treatment comprises three doses of an anti-clusterin antibody or antigen binding fragment thereof and one dose of taxane such as docetaxel. In exemplary embodiments, the initial cycle of treatment comprises four doses of an anti-clusterin antibody or antigen binding fragment thereof and one dose of taxane such as docetaxel.

In some embodiments, the subsequent cycle of treatment comprises at least one dose of an anti-clusterin antibody or antigen binding fragment thereof and at least one dose of taxane such as docetaxel. In exemplary embodiments, the subsequent cycle of treatment comprises two doses of an anti-clusterin antibody or antigen binding fragment thereof and one dose of taxane such as docetaxel. In exemplary embodiments, the subsequent cycle of treatment comprises three doses of an anti-clusterin antibody or antigen binding fragment thereof and one dose of taxane such as docetaxel. In exemplary embodiments, the subsequent cycle of treatment comprises four doses of an anti-clusterin antibody or antigen binding fragment thereof and one dose of taxane such as docetaxel.

In some embodiments, the initial cycle of treatment and subsequent cycle of treatment each comprise at least one dose of the anti-clusterin antibody or antigen binding fragment thereof and at least one dose of docetaxel.

In some embodiments, one cycle of treatment (initial cycle of treatment and/or subsequent cycle of treatment) is approximately 21 days. In other embodiments, one cycle of treatment is more than 21 days, such as for example, approximately one month or more (e.g., approximately 28-31 days). In other embodiments, one cycle of treatment is less than 21 days, such as for example, approximately 2 weeks (e.g., approximately 13-16 days).

In accordance with the present disclosure the subject is treated for one or more cycles of treatment.

In some embodiments, one cycle of treatment may last approximately 21 days and may include administration of the anti-clusterin antibody or antigen binding fragment thereof once every three weeks (approximately) and administration of docetaxel once every three weeks (approximately). In such a case, one cycle of treatment therefore comprises administration of one dose of the anti-clusterin antibody or antigen binding fragment thereof and one dose of docetaxel every 21 days (approximately).

In some embodiments, one cycle of treatment may last approximately 14 days and may include administration of the anti-clusterin antibody or antigen binding fragment thereof once every two weeks (approximately) and administration of docetaxel once every two weeks (approximately). In such a case, one cycle of treatment therefore comprises administration of one dose of the anti-clusterin antibody or antigen binding fragment thereof and one dose of docetaxel every 14 days (approximately).

In some embodiments, one cycle of treatment may last approximately 1 month and may include administration of the anti-clusterin antibody or antigen binding fragment thereof once per month (approximately) and administration of docetaxel once per month (approximately). In such a case, one cycle of treatment therefore comprises administration of one dose of the anti-clusterin antibody or antigen binding fragment thereof and one dose of docetaxel every month (approximately).

The use of the same administration interval for both drugs is more convenient and may be beneficial for compliance as both drugs may be administered on same day.

In some embodiments, one cycle of treatment is approximately 21 days and consists in administration of the anti-clusterin antibody or antigen binding fragment thereof once weekly and administration of docetaxel once every three weeks.

In some embodiments, the initial cycle of treatment and/or subsequent cycle of treatment may each independently comprise administering the anti-clusterin antibody or antigen binding fragment thereof at a dose of approximately 3 mg/kg to approximately 20 mg/kg once per week, once every two weeks, or once every three weeks and administration of docetaxel at a dose of approximately 50 mg/m² to 100 mg/m² once every two weeks or once every three weeks.

In an exemplary embodiment, the method comprises administering docetaxel at a dose of approximately 75 mg/m² once every three weeks and administering the anti-clusterin antibody or antigen binding fragment thereof per week, once every two weeks or once every three weeks.

In another exemplary embodiment, the method comprises administering the anti-clusterin antibody or antigen binding fragment thereof at a dose of approximately 12 mg/kg once per week and administering docetaxel at a dose of approximately 75 mg/m² once every three weeks.

In yet another exemplary embodiment, the method comprises administering the anti-clusterin antibody or antigen binding fragment thereof at a dose of approximately 12 mg/kg once every three weeks and administering docetaxel at a dose of approximately 75 mg/m² once every three weeks.

In some embodiments, the method of the present disclosure may comprise administering to a subject in need,
a) at least one initial cycle of treatment comprising administering the anti-clusterin antibody or antigen binding fragment thereof at a dose of approximately 3 mg/kg to approximately 20 mg/kg once per week and administration of docetaxel at a dose of approximately 50 mg/m² to 100 mg/m² once every three weeks and;
b) at least one subsequent cycle of treatment comprising administering the anti-clusterin antibody or antigen binding fragment thereof at a dose of approximately 3 mg/kg to approximately 20 mg/kg once every three weeks and administration of docetaxel at a dose of approximately 50 mg/m² to 100 mg/m² once every three weeks.

In other embodiments, the method of the present disclosure may comprise administering to a subject in need,
a) at least one initial cycle of treatment comprising administering the anti-clusterin antibody or antigen binding fragment thereof at a dose of approximately 12 mg/kg once per week and administration of docetaxel at a dose of approximately 75 mg/m² once every three weeks and;
b) at least one subsequent cycle of treatment comprising administering the anti-clusterin antibody or antigen binding fragment thereof at a dose of approximately 3 mg/kg to approximately 20 mg/kg once every three weeks and administration of docetaxel at a dose of approximately 75 mg/m² once every three weeks.

In yet other embodiments, the method of the present disclosure may comprise administering to a subject in need,
a) at least one initial cycle of treatment comprising administering the anti-clusterin antibody or antigen binding fragment thereof at a dose of approximately 12 mg/kg once per week and administration of docetaxel at a dose of approximately 75 mg/m² once every three weeks and;
b) at least one subsequent cycle of treatment comprising administering the anti-clusterin antibody or antigen binding fragment thereof at a dose of approximately 12 mg/kg once every three weeks and administration of docetaxel at a dose of approximately 75 mg/m² once every three weeks.

In an exemplary embodiment, the method comprises administering docetaxel at a dose of approximately 50 mg/m² once every two weeks and administering the anti-clusterin antibody or antigen binding fragment thereof once per week, once every two weeks or once every three weeks.

In another exemplary embodiment, the method comprises administering the anti-clusterin antibody or antigen binding fragment thereof at a dose of approximately 12 mg/kg once per week and administering docetaxel at a dose of approximately 50 mg/m² once every two weeks.

In yet another exemplary embodiment, the method comprises administering the anti-clusterin antibody or antigen binding fragment thereof at a dose of approximately 12 mg/kg once every two weeks and administering docetaxel at a dose of approximately 50 mg/m² once every two weeks.

In yet other embodiments, the method of the present disclosure may comprise administering to a subject in need,
a) at least one initial cycle of treatment comprising administering the anti-clusterin antibody or antigen binding fragment thereof at a dose of approximately 12 mg/kg once per week and administration of docetaxel at a dose of approximately 50 mg/m² once every two weeks and;
b) at least one subsequent cycle of treatment comprising administering the anti-clusterin antibody or antigen binding fragment thereof at a dose of approximately 3 mg/kg to approximately 20 mg/kg once every two weeks and administration of docetaxel at a dose of approximately 50 mg/m² once every two weeks.

In yet other embodiments, the method of the present disclosure may comprise administering to a subject in need,
a) at least one initial cycle of treatment comprising administering the anti-clusterin antibody or antigen binding fragment thereof at a dose of approximately 12 mg/kg once per week and administration of docetaxel at a dose of approximately 50 mg/m² once every two weeks and;
b) at least one subsequent cycle of treatment comprising administering the anti-clusterin antibody or antigen binding fragment thereof at a dose of approximately 12 mg/kg once every two weeks and administration of docetaxel at a dose of approximately 50 mg/m² once every two weeks.

In an exemplary embodiment, the subject is treated for at least one cycle of treatment. In an exemplary embodiment, the subject is treated for at least one initial cycle of treatment and at least one subsequent cycle of treatment.

In another exemplary embodiment, the subject is treated for at least two cycles of treatment. In an exemplary embodiment, the subject is treated for at least two initial cycles of treatment. In an exemplary embodiment, the subject is treated for at least two subsequent cycles of treatment. In an exemplary embodiment, the subject is treated for at least one initial cycle of treatment and at least two subsequent cycles of treatment. In an exemplary embodiment, the subject is treated for at least two initial cycles of treatment and at least two subsequent cycles of treatment. In an exemplary embodiment, the subject is treated for at least two initial cycles of treatment and more than two subsequent cycles of treatment. In an exemplary embodiment, the subject is treated for more than two initial cycles of treatment and more than two subsequent cycles of treatment.

In an additional exemplary embodiment, the subject is treated for at least three cycles of treatment. In an exemplary embodiment, the subject is treated for at least three initial cycles of treatment. In an exemplary embodiment, the subject is treated for at least three subsequent cycles of treatment. In an exemplary embodiment, the subject is treated for at least three initial cycles of treatment and at least three subsequent cycles of treatment. In an exemplary embodiment, the subject is treated for at least three initial cycles of treatment and more than three subsequent cycles of treatment.

In yet an additional exemplary embodiment, the subject is treated for at least four cycles of treatment. In an exemplary embodiment, the subject is treated for at least four initial cycles of treatment. In an exemplary embodiment, the subject is treated for at least four subsequent cycles of treatment. In an exemplary embodiment, the subject is treated for at least four initial cycles of treatment and at least four subsequent cycles of treatment. In an exemplary embodiment, the subject is treated for at least four initial cycles of treatment and more than four subsequent cycles of treatment.

In other exemplary embodiments, the subject is treated or receives four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more treatment cycles. In yet another exemplary embodiment, the subject is treated or receives four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more initial cycles of treatment. In yet a further exemplary embodiment, the subject is treated or receives four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more initial and/or subsequent cycles of treatment. In some embodiments, the treatment cycles are consecutive.

In some embodiments, the treatment cycles are uninterrupted.

In some embodiments, the treatment cycles are interrupted.

In some embodiments, the treatment cycles are interrupted for a period of time (ranging from one day to several weeks or months). In some embodiments, at least one treatment cycle is interrupted. In other embodiments, more than one treatment cycles are interrupted. In other embodiments, the treatment is interrupted after a certain period of time determined by a physician or clinician.

In accordance with the present disclosure the combination therapy is administered for at least two consecutive cycles.

In accordance with the present disclosure the combination therapy is administered for at least three consecutive cycles.

In accordance with the present disclosure the combination therapy is administered for at least four consecutive cycles.

In accordance with the present disclosure the combination therapy is administered for at least five consecutive cycles.

In accordance with the present disclosure the combination therapy is administered for at least six consecutive cycles.

In accordance with the present disclosure the combination therapy is administered for at least seven consecutive cycles.

In accordance with the present disclosure the combination therapy is administered for at least eight consecutive cycles.

In accordance with the present disclosure the combination therapy is administered for at least nine consecutive cycles.

In accordance with the present disclosure the combination therapy is administered for at least ten consecutive cycles.

In accordance with the present disclosure the combination therapy is administered for at least eleven consecutive cycles.

In accordance with the present disclosure the combination therapy is administered for at least twelve consecutive cycles.

In some embodiments, a clinical benefit may be observed or measured after two cycles of treatment.

In some embodiments, a clinical benefit may be observed or measured after three cycles of treatment.

In some embodiments, a clinical benefit may be observed or measured after four cycles of treatment.

In some embodiments, a clinical benefit may be observed or measured after five cycles of treatment.

In some embodiments, a clinical benefit may be observed or measured after more than five cycles of treatment.

In some embodiments, the combination therapy is administered as long as a clinical benefit is observed or measured.

In some embodiments, the combination therapy is administered until tumor progression.

In some embodiments, the combination therapy is administered until unmanageable toxicity occurs.

In some embodiments, administration of the anti-clusterin antibody or antigen binding fragment thereof is maintained after the appearance of clinical benefits.

The present disclosure also provides a kit comprising one or more containers comprising at least one dose of an anti-clusterin antibody or antigen binding fragment thereof, one or more containers comprising at least one dose of a taxane such as, for example, docetaxel for use in combination therapy and a package insert comprising instructions for treating a subject as described herein.

In some embodiments, the package insert states that the combination therapy is intended for treatment of a subject having NSCLC or metastatic NSCLC.

### BRIEF DESCRIPTION OF THE DRAWINGS

FIG. 1: table showing the percentage of reduction of target lesions, the duration of patients on treatment as the best response as per RECIST 1.1 (PT; Patient, BR; Best response, PD-L1; PD-L1 tumor proportion score, PR; Partial response, SD; Stable disease, PD; progressive disease, AE; adverse event).

FIG. 2: histology analyses of pre-(a and c) and on-treatment (b and d) biopsies from two patients under study. Tumor sections were stained with hematoxylin and eosin.

Further scope, applicability and advantages of the present disclosure will become apparent from the non-restrictive detailed description given hereinafter. It should be understood, however, that this detailed description, while indicating exemplary embodiments of the disclosure, is given by way of example only, with reference to the accompanying drawings.

### DETAILED DESCRIPTION

### Definitions

Unless indicated otherwise, the amino acid numbering indicated for the dimerization domain are in accordance with the EU numbering system.

The use of the terms “a” and “an” and “the” and similar referents in the context of describing embodiments (especially in the context of the claims) are to be construed to cover both the singular and the plural, unless otherwise indicated herein or clearly contradicted by context.

Unless specifically stated or obvious from context, as used herein the term “or” is understood to be inclusive and covers both “or” and “and”.

The term “and/or” where used herein is to be taken as specific disclosure of each of the specified features or components with or without the other.

The terms “comprising”, “having”, “including”, and “containing” are to be construed as open-ended terms (i.e., meaning “including, but not limited to”) unless otherwise noted. The term “consisting of” is to be construed as close-ended.

The term “subject” refers to a human or any non-human animal (e.g., mouse, rat, rabbit, dog, cat, cattle, sheep, horse or primate) having lung cancer such as without limitation, a non-small cell lung cancer, metastatic non-small cell lung cancer and/or a subject having stage III to IV non-small cell lung cancer. The term “subject” is used herein interchangeably with “individual” or “patient.”

The term “antibody” is used in the broadest sense and encompasses various antibody formats and structures, including any immunoglobulin, monoclonal antibody, polyclonal antibody, bivalent antibody, monovalent antibody, bispecific antibody, multiple specific (multi-specific) antibody, conventional antibody, single domain antibody, single chain antibody, heavy chain only antibody, nanobody, full-length antibody, humanized antibody, chimeric antibody that binds to a specific antigen, and any antigen binding fragment that exhibits the desired antigen binding activity. An antibody can be naturally occurring (native) or the results or recombination technologies. As used herein the term “conventional antibody” has the same format as a naturally occurring human antibody. It is to be understood herein that the sequence of a conventional antibody may be partially and/or totally derived from a non-human animal antibody or from a human antibody and therefore encompasses without limitations, monoclonal, chimeric, human and humanized antibody.

The term “treat” or “treating” for purposes of this disclosure refers to the action of providing a treatment (such as the combination therapy disclosed herein) to a subject. As used herein, the term “treat,” or “treating” refers to any method used to partially or completely alleviate, ameliorate, relieve, inhibit, prevent, delay onset of, reduce severity, reduce incidence, stabilize, and/or manage one or more symptoms and/or features of a particular disease, disorder, and/or condition and/or to obtain or maintain a clinical benefit as defined per RECIST 1.1 guidelines. It is to be understood that a treatment may be effective in some subjects having the particular disease, disorder, and/or condition but have no effects in others that have nevertheless received treatment.

The term “therapeutically effective” refers to a treatment that results in partial or complete alleviation, amelioration, relief, inhibition, prevention, delay in onset, reduction of severity, reduction in incidence, stabilization and/or improved management of one or more symptoms and/or features of a particular disease, disorder, and/or condition, and/or that results in a clinical benefit as defined per RECIST 1.1 guidelines for some subjects.

The term “EMT signature” as used herein refers to changes that are indicative of a loss of epithelial phenotype and/or acquisition of a mesenchymal phenotype that are observable at the cellular level and/or observable or measurable at the genetic level or protein level.

The term “about” or “approximately” with respect to a given value means that variation in the value is contemplated. In some embodiments, the term “about” or “approximately” shall generally mean a range within +/−20 percent, within +/−10 percent, within +/−5 percent, within +/−4 percent, within +/−3 percent, within +/−2 percent or within +/−1 percent of a given value or range.

The expression “over the entire course of the treatment period” means that both the anti-clusterin antibody or antigen binding fragments and docetaxel are administered at each treatment cycle.

The term “essentially” is used to characterize an action that is carried out most of the time or a state that occurs most of the time. For example, the expression “essentially over the entire course of the treatment period” means that both the anti-clusterin antibody or antigen binding fragments and docetaxel are administered at each treatment cycle and during the entire treatment period but occasionally a dose of either of the anti-clusterin antibody or antigen binding fragments or docetaxel or a dose of each may be intentionally or non-intentionally missed.

The term “functional immune system” with respect to a subject means that the immune system of the subject is essentially not affected by cancer or by medication, that the subject is immunocompetent or that the subject is not immunosuppressed.

The expression “treatment comprising an immune checkpoint antibody” refers to treatment with an immune checkpoint antibody as monotherapy or in combination therapy.

The expression “adequate organ and immune function” refers to one or more of the parameters provided in Table 1.

### Exemplary Embodiments of Combination Therapies, Methods, Uses, Dosages, Regimens and Schedules

The present disclosure provides a combination therapy that includes an anti-clusterin antibody or antigen binding fragment thereof and a taxane.

More particularly, the present disclosure provides a combination therapy that includes an anti-clusterin antibody or antigen binding fragment thereof and a docetaxel.

The present disclosure provides a method of treating a subject having lung cancer (e.g., lung carcinoma. with the combination therapy disclosed herein.

More particularly, the present disclosure provides a method of treating a subject having non-small cell lung cancer with the combination therapy disclosed herein.

More specifically, the present disclosure provides a method of treating a subject having metastatic non-small cell lung cancer with the combination therapy disclosed herein.

The present disclosure also relates to a method of treating a subject having non-small cell lung cancer with the combination therapy disclosed herein so as to obtain a clinical benefit.

The present disclosure further relates to a method of treating a subject having metastatic non-small cell lung cancer with the combination therapy disclosed herein so as to obtain a clinical benefit.

The present disclosure also relates to a method of procuring a clinical benefit to a subject having non-small cell lung cancer with the combination therapy disclosed herein. The present disclosure further relates to a method of procuring a clinical benefit to a subject having metastatic non-small cell lung cancer with the combination therapy disclosed herein.

In some embodiments, the method may comprise administering the combination therapy in a dosage sufficient to result in a clinical benefit.

In some embodiments, the method may comprise administering the combination therapy for period of time sufficient to result in a clinical benefit.

In some embodiments, the method may comprise administering the combination therapy according to a schedule of treatment sufficient to result in a clinical benefit.

In some embodiments, the method may comprise administering the combination therapy according to a treatment regimen sufficient to result in a clinical benefit.

Accordingly, in certain aspects, the method of the present disclosure comprises administering a combination therapy comprising an anti-clusterin antibody or antigen binding fragment thereof and a taxane.

In other aspects, the method of the present disclosure comprises administering a combination therapy comprising an anti-clusterin antibody or antigen binding fragment thereof and docetaxel.

In accordance with the present disclosure, the anti-clusterin antibody or antigen binding fragment thereof is administered at a therapeutically effective dose.

In accordance with the present disclosure, the anti-clusterin antibody or antigen binding fragment thereof is administered at a safe dose.

In accordance with the present disclosure, docetaxel is administered at a therapeutically effective dose.

In accordance with the present disclosure, docetaxel is administered at a safe dose.

In some embodiments, docetaxel is administered at a dose sufficient to allow chemotherapy-induced immunogenic modulation of tumor.

In accordance with the present disclosure, docetaxel is administered at an administration interval sufficient to allow chemotherapy-induced immunogenic modulation of tumor.

In some embodiments, the method comprises administering a combination therapy comprising an anti-clusterin antibody or antigen binding fragment thereof at a dose of approximately 3 mg/kg to approximately 20 mg/kg and docetaxel at a dose of approximately 60 mg/m² to 100 mg/m².

In accordance with the present disclosure, the anti-clusterin antibody or antigen binding fragment thereof is administered at a dose of between approximately 3 mg/kg and approximately 20 mg/kg, such as for example, between approximately 4 mg/kg and approximately 20 mg/kg, between approximately 5 mg/kg and approximately 20 mg/kg, between approximately 6 mg/kg and approximately 20 mg/kg, between approximately 6 mg/kg and approximately 18 mg/kg, between approximately 6 mg/kg and approximately 17 mg/kg, between approximately 6 mg/kg and approximately 16 mg/kg, between approximately 6 mg/kg and approximately 15 mg/kg, between approximately 6 mg/kg and approximately 14 mg/kg, between approximately 6 mg/kg and approximately 13 mg/kg, between approximately 6 mg/kg and approximately 12 mg/kg, between approximately 7 mg/kg and approximately 18 mg/kg, between approximately 7 mg/kg and approximately 17 mg/kg, between approximately 7 mg/kg and approximately 16 mg/kg, between approximately 7 mg/kg and approximately 15 mg/kg, between approximately 7 mg/kg and approximately 14 mg/kg, between approximately 7 mg/kg and approximately 13 mg/kg, between approximately 7 mg/kg and approximately 12 mg/kg, between approximately 8 mg/kg and approximately 18 mg/kg, between approximately 8 mg/kg and approximately 17 mg/kg, between approximately 8 mg/kg and approximately 16 mg/kg, between approximately 8 mg/kg and approximately 15 mg/kg, between approximately 8 mg/kg and approximately 14 mg/kg, between approximately 8 mg/kg and approximately 13 mg/kg, between approximately 8 mg/kg and approximately 12 mg/kg, between approximately 9 mg/kg and approximately 18 mg/kg, between approximately 9 mg/kg and approximately 17 mg/kg, between approximately 9 mg/kg and approximately 16 mg/kg, between approximately 9 mg/kg and approximately 15 mg/kg, between approximately 9 mg/kg and approximately 14 mg/kg, between approximately 9 mg/kg and approximately 13 mg/kg, between approximately 9 mg/kg and approximately 12 mg/kg, between approximately 10 mg/kg and approximately 18 mg/kg, between approximately 10 mg/kg and approximately 17 mg/kg, between approximately 10 mg/kg and approximately 16 mg/kg, between approximately 10 mg/kg and approximately 15 mg/kg, between approximately 10 mg/kg and approximately 14 mg/kg, between approximately 10 mg/kg and approximately 13 mg/kg, or between approximately 10 mg/kg and approximately 12 mg/kg.

In some embodiments, the anti-clusterin antibody or antigen binding fragment thereof is administered at a dose of approximately 3.0 mg/kg.

In some embodiments, the anti-clusterin antibody or antigen binding fragment thereof is administered at a dose of 3.0 mg/kg.

In some embodiments, the anti-clusterin antibody or antigen binding fragment thereof is administered at a dose of approximately 4.0 mg/kg.

In some embodiments, the anti-clusterin antibody or antigen binding fragment thereof is administered at a dose of 4.0 mg/kg.

In some embodiments, the anti-clusterin antibody or antigen binding fragment thereof is administered at a dose of approximately 5.0 mg/kg.

In some embodiments, the anti-clusterin antibody or antigen binding fragment thereof is administered at a dose of 5.0 mg/kg.

In some embodiments, the anti-clusterin antibody or antigen binding fragment thereof is administered at a dose of approximately 6.0 mg/kg.

In some embodiments, the anti-clusterin antibody or antigen binding fragment thereof is administered at a dose of 6.0 mg/kg.

In some embodiments, the anti-clusterin antibody or antigen binding fragment thereof is administered at a dose of approximately 7.0 mg/kg.

In some embodiments, the anti-clusterin antibody or antigen binding fragment thereof is administered at a dose of 7.0 mg/kg.

In some embodiments, the anti-clusterin antibody or antigen binding fragment thereof is administered at a dose of approximately 8.0 mg/kg.

In some embodiments, the anti-clusterin antibody or antigen binding fragment thereof is administered at a dose of 8.0 mg/kg.

In some embodiments, the anti-clusterin antibody or antigen binding fragment thereof is administered at a dose of approximately 9.0 mg/kg.

In some embodiments, the anti-clusterin antibody or antigen binding fragment thereof is administered at a dose of 9.0 mg/kg.

In some embodiments, the anti-clusterin antibody or antigen binding fragment thereof is administered at a dose of approximately 10.0 mg/kg.

In some embodiments, the anti-clusterin antibody or antigen binding fragment thereof is administered at a dose of 10.0 mg/kg.

In some embodiments, the anti-clusterin antibody or antigen binding fragment thereof is administered at a dose of approximately 11.0 mg/kg.

In some embodiments, the anti-clusterin antibody or antigen binding fragment thereof is administered at a dose of 11.0 mg/kg.

In some embodiments, the anti-clusterin antibody or antigen binding fragment thereof is administered at a dose of approximately 12.0 mg/kg.

In some embodiments, the anti-clusterin antibody or antigen binding fragment thereof is administered at a dose of 12.0 mg/kg.

In some embodiments, the anti-clusterin antibody or antigen binding fragment thereof is administered at a dose of approximately 13.0 mg/kg.

In some embodiments, the anti-clusterin antibody or antigen binding fragment thereof is administered at a dose of 13.0 mg/kg.

In some embodiments, the anti-clusterin antibody or antigen binding fragment thereof is administered at a dose of approximately 14.0 mg/kg.

In some embodiments, the anti-clusterin antibody or antigen binding fragment thereof is administered at a dose of 14.0 mg/kg.

In some embodiments, the anti-clusterin antibody or antigen binding fragment thereof is administered at a dose of approximately 15.0 mg/kg.

In some embodiments, the anti-clusterin antibody or antigen binding fragment thereof is administered at a dose of 15.0 mg/kg.

In some embodiments, the anti-clusterin antibody or antigen binding fragment thereof is administered at a dose of approximately 16.0 mg/kg.

In some embodiments, the anti-clusterin antibody or antigen binding fragment thereof is administered at a dose of 16.0 mg/kg.

In some embodiments, the anti-clusterin antibody or antigen binding fragment thereof is administered at a dose of approximately 17.0 mg/kg.

In some embodiments, the anti-clusterin antibody or antigen binding fragment thereof is administered at a dose of 17.0 mg/kg.

In some embodiments, the anti-clusterin antibody or antigen binding fragment thereof is administered at a dose of approximately 18.0 mg/kg.

In some embodiments, the anti-clusterin antibody or antigen binding fragment thereof is administered at a dose of 18.0 mg/kg.

In some embodiments, the anti-clusterin antibody or antigen binding fragment thereof is administered at a dose of approximately 19.0 mg/kg.

In some embodiments, the anti-clusterin antibody or antigen binding fragment thereof is administered at a dose of 19.0 mg/kg.

In some embodiments, the anti-clusterin antibody or antigen binding fragment thereof is administered at a dose of approximately 20.0 mg/kg.

In some embodiments, the anti-clusterin antibody or antigen binding fragment thereof is administered at a dose of 20.0 mg/kg.

In accordance with the present disclosure docetaxel is administered at a dose of between approximately 50 mg/m² to approximately 100 mg/m² such as for example, 60 mg/m² to approximately 100 mg/m², between approximately 60 mg/m² to approximately 95 mg/m², between approximately 60 mg/m² to approximately 90 mg/m², between approximately 60 mg/m² to approximately 85 mg/m², between approximately 60 mg/m² to approximately 80 mg/m², between approximately 50 mg/m² to approximately 75 mg/m², between approximately 60 mg/m² to approximately 75 mg/m², between approximately 75 mg/m² to approximately 95 mg/m², between approximately 75 mg/m² to approximately 90 mg/m², between approximately 75 mg/m² to approximately 85 mg/m², between approximately 75 mg/m² to approximately 80 mg/m², between approximately 70 mg/m² to approximately 95 mg/m², between approximately 70 mg/m² to approximately 90 mg/m², between approximately 70 mg/m² to approximately 85 mg/m², between approximately 70 mg/m² to approximately 80 mg/m², or between approximately 70 mg/m² to approximately 75 mg/m².

In some embodiments, docetaxel is administered at a dose of approximately 50 mg/m².

In some embodiments, docetaxel is administered at a dose of approximately 60 mg/m².

In some embodiments, docetaxel is administered at a dose of approximately 65 mg/m².

In some embodiments, docetaxel is administered at a dose of approximately 70 mg/m².

In some embodiments, docetaxel is administered at a dose of approximately 75 mg/m².

In some embodiments, docetaxel is administered at a dose of approximately 80 mg/m².

In some embodiments, docetaxel is administered at a dose of approximately 85 mg/m².

In some embodiments, docetaxel is administered at a dose of approximately 90 mg/m².

In some embodiments, docetaxel is administered at a dose of approximately 95 mg/m².

In some embodiments, docetaxel is administered at a dose of approximately 100 mg/m².

An exemplary embodiment of a method of the present disclosure is a method of treating a subject having non-small cell lung cancer such as, for example, metastatic non-small cell lung cancer comprising administering a combination therapy comprising an anti-clusterin antibody or antigen binding fragment thereof and docetaxel, where the anti-clusterin antibody or antigen binding fragment thereof is administered at a dose of approximately 3 mg/kg to approximately 20 mg/kg and comprises a light chain variable region comprising the complementarity determining regions (CDRs) of the light chain variable region set forth in SEQ ID NO:12 and a heavy chain variable region comprising the CDRs of the heavy chain variable region set forth in SEQ ID NO:13 and docetaxel is administered at a dose of approximately 50 mg/m² to approximately 100 mg/m².

Another exemplary embodiment of a method of the present disclosure is a method of treating a subject having non-small cell lung cancer such as, for example, metastatic non-small cell lung cancer comprising administering a combination therapy comprising an anti-clusterin antibody or antigen binding fragment thereof and docetaxel, where the anti-clusterin antibody or antigen binding fragment thereof is administered at a dose of approximately 12 mg/kg and comprises a light chain variable region comprising the complementarity determining regions (CDRs) of the light chain variable region set forth in SEQ ID NO:12 and a heavy chain variable region comprising the CDRs of the heavy chain variable region set forth in SEQ ID NO:13 and docetaxel is administered at a dose of approximately 50 mg/m² to approximately 75 mg/m².

Another exemplary embodiment of a method of the present disclosure is a method of treating a subject having non-small cell lung cancer such as, for example, metastatic non-small cell lung cancer comprising administering a combination therapy comprising an anti-clusterin antibody or antigen binding fragment thereof and docetaxel, where the anti-clusterin antibody or antigen binding fragment thereof is administered at a dose of approximately 12 mg/kg and comprises a light chain variable region comprising the amino acid sequence set forth in SEQ ID NO:12 and a heavy chain variable region comprising the amino acid sequence set forth in SEQ ID NO:13 and docetaxel is administered at a dose of approximately 50 mg/m² to approximately 75 mg/m².

Yet another exemplary embodiment of a method of the present disclosure is a method of treating a subject having non-small cell lung cancer such as, for example, metastatic non-small cell lung cancer comprising administering a combination therapy comprising an anti-clusterin antibody or antigen binding fragment thereof and docetaxel so as to obtain a clinical benefit, where the anti-clusterin antibody or antigen binding fragment thereof is administered at a dose of approximately 3 mg/kg to approximately 20 mg/kg and comprises a light chain variable region comprising the complementarity determining regions (CDRs) of the light chain variable region set forth in SEQ ID NO:12 and a heavy chain variable region comprising the CDRs of the heavy chain variable region set forth in SEQ ID NO:13 and docetaxel is administered at a dose of approximately 50 mg/m² to approximately 100 mg/m².

A further exemplary embodiment of a method of the present disclosure is a method of treating a subject having non-small cell lung cancer such as, for example, metastatic non-small cell lung cancer comprising administering a combination therapy comprising an anti-clusterin antibody or antigen binding fragment thereof and docetaxel so as to obtain a clinical benefit, where the anti-clusterin antibody or antigen binding fragment thereof is administered at a dose of approximately 12 mg/kg and comprises a light chain variable region comprising the complementarity determining regions (CDRs) of the light chain variable region set forth in SEQ ID NO:12 and a heavy chain variable region comprising the CDRs of the heavy chain variable region set forth in SEQ ID NO:13 and docetaxel is administered at a dose of approximately 50 mg/m² to approximately 75 mg/m².

Yet a further exemplary embodiment of a method of the present disclosure is a method of treating a subject having non-small cell lung cancer such as, for example, metastatic non-small cell lung cancer comprising administering a combination therapy comprising an anti-clusterin antibody or antigen binding fragment thereof and docetaxel so as to obtain a clinical benefit, where the anti-clusterin antibody or antigen binding fragment thereof is administered at a dose of approximately 12 mg/kg and comprises a light chain variable region comprising the amino acid sequence set forth in SEQ ID NO: 12 and a heavy chain variable region comprising the amino acid sequence set forth in SEQ ID NO:13 and docetaxel is administered at a dose of approximately 50 mg/m² to approximately 75 mg/m².

In some embodiments, the anti-clusterin antibody or antigen binding fragment thereof is administered once weekly.

In some embodiments, the anti-clusterin antibody or antigen binding fragment thereof is administered twice weekly.

In some embodiments, the anti-clusterin antibody or antigen binding fragment thereof is administered once every two weeks.

In some embodiments, the anti-clusterin antibody or antigen binding fragment thereof is administered once every three weeks.

In some embodiments, the anti-clusterin antibody or antigen binding fragment thereof is administered once every four weeks.

In some embodiments, docetaxel is administered every week.

In some embodiments, docetaxel is administered every two weeks.

In some embodiments, docetaxel is administered every three weeks.

In some embodiments, docetaxel is administered every four weeks.

In some embodiments, docetaxel is administered once per week.

In some embodiments, docetaxel is administered once every two weeks.

In some embodiments, docetaxel is administered once every three weeks.

In some embodiments, docetaxel is administered once every four weeks.

In some embodiments, docetaxel is administered once every five weeks.

In some embodiments, docetaxel is administered once every six weeks.

In some embodiments, a treatment cycle is considered completed after a period of approximately seven days after a subject has received both the anti-clusterin antibody or antigen binding fragment thereof and docetaxel.

In some exemplary embodiments, one treatment cycle is approximately 7 days. In some exemplary embodiments, essentially all treatment cycles are approximately 7 days.

In some exemplary embodiments, each treatment cycles are approximately 7 days.

In accordance with the present disclosure, the subject may thus receive a treatment cycle every 7 days.

In some exemplary embodiments, one treatment cycle is approximately 14 days. In some exemplary embodiments, essentially all treatment cycles are approximately 14 days.

In some exemplary embodiments, each treatment cycles are approximately 14 days. In accordance with the present disclosure, the subject may thus receive a treatment cycle every 14 days.

In some exemplary embodiments, one treatment cycle is approximately 21 days. In some exemplary embodiments, essentially all treatment cycles are approximately 21 days.

In some exemplary embodiments, each treatment cycles are approximately 21 days. In accordance with the present disclosure, the subject may thus receive a treatment cycle every 21 days.

In some exemplary embodiments, one treatment cycle is approximately 28 days. In some exemplary embodiments, essentially all treatment cycles are approximately 28 days.

In some exemplary embodiments, each treatment cycles are approximately 28 days.

In accordance with the present disclosure, the subject may thus receive a treatment cycle every 28 days.

In an exemplary embodiment, the combination therapy is administered in one or more cycles of treatment including administration of the anti-clusterin antibody or antigen binding fragment thereof on days 1, 8 and 15 and administration of docetaxel on day 1.

It is to be understood that the interval of administration of the anti-clusterin antibody or antigen binding fragment thereof and docetaxel may be changed without significantly changing the effect of the combination therapy.

For example, the anti-clusterin antibody or antigen binding fragment thereof may be administered on days 1, 7 and 14 and docetaxel may be administered on day 1.

In another example, the anti-clusterin antibody or antigen binding fragment thereof may be administered on days 1, 7 and 16 and docetaxel may be administered on day 1. In yet another example, the anti-clusterin antibody or antigen binding fragment thereof may be administered on days 1, 8 and 16 and docetaxel may be administered on day 1.

In an exemplary embodiment, one treatment cycle consists in administration of the anti-clusterin antibody or antigen binding fragment thereof once per week and administration of docetaxel every three weeks.

In an exemplary embodiment, one treatment cycle consists in administration of the anti-clusterin antibody or antigen binding fragment thereof every two weeks and administration of docetaxel every two weeks.

In an exemplary embodiment, one treatment cycle consists in administration of the anti-clusterin antibody or antigen binding fragment thereof every two weeks and administration of docetaxel every three weeks.

In an exemplary embodiment, one treatment cycle consists in administration of the anti-clusterin antibody or antigen binding fragment thereof every three weeks and administration of docetaxel every three weeks.

In an exemplary embodiment, one treatment cycle consists in administration of the anti-clusterin antibody or antigen binding fragment thereof once per week and administration of docetaxel every month.

In an exemplary embodiment, one treatment cycle consists in administration of the anti-clusterin antibody or antigen binding fragment thereof every two weeks and administration of docetaxel every month.

In an exemplary embodiment, one treatment cycle consists in administration of the anti-clusterin antibody or antigen binding fragment thereof every month and administration of docetaxel every month.

In an exemplary embodiment, one treatment cycle consists in administration of the anti-clusterin antibody or antigen binding fragment thereof every two months and administration of docetaxel every two months.

In an exemplary embodiment, one treatment cycle consists in administration of the anti-clusterin antibody or antigen binding fragment thereof every three months and administration of docetaxel every three months.

In some embodiments, the method comprises administering a combination therapy comprising an anti-clusterin antibody or antigen binding fragment thereof at a dose of approximately 3 mg/kg to approximately 20 mg/kg and docetaxel at a dose of approximately 50 mg/m² to approximately 100 mg/m² every two to three weeks.

In exemplary embodiments, the anti-clusterin antibody or antigen binding fragment thereof is administered at a dose of approximately 12 mg/kg and docetaxel is administered at a dose of approximately 75 mg/m² every three weeks.

In exemplary embodiments the anti-clusterin antibody or antigen binding fragment thereof is administered at a dose of approximately 12 mg/kg and docetaxel is administered at a dose of approximately 60 mg/m² every three weeks.

In exemplary embodiments the anti-clusterin antibody or antigen binding fragment thereof is administered at a dose of approximately 12 mg/kg and docetaxel is administered at a dose of approximately 50 mg/m² every two weeks.

In exemplary embodiments the anti-clusterin antibody or antigen binding fragment thereof is administered at a dose of approximately 9 mg/kg and docetaxel is administered at a dose of approximately 75 mg/m² every three weeks.

In exemplary embodiments the anti-clusterin antibody or antigen binding fragment thereof is administered at a dose of approximately 9 mg/kg and docetaxel is administered at a dose of approximately 60 mg/m² every three weeks.

In exemplary embodiments the anti-clusterin antibody or antigen binding fragment thereof is administered at a dose of approximately 9 mg/kg and docetaxel is administered at a dose of approximately 50 mg/m² every two weeks.

In exemplary embodiments the anti-clusterin antibody or antigen binding fragment thereof is administered at a dose of approximately 6 mg/kg and docetaxel is administered at a dose of approximately 75 mg/m² every three weeks.

In exemplary embodiments the anti-clusterin antibody or antigen binding fragment thereof is administered at a dose of approximately 6 mg/kg and docetaxel is administered at a dose of approximately 60 mg/m² every three weeks.

In exemplary embodiments the anti-clusterin antibody or antigen binding fragment thereof is administered at a dose of approximately 6 mg/kg and docetaxel is administered at a dose of approximately 50 mg/m² every two weeks.

In exemplary embodiments the anti-clusterin antibody or antigen binding fragment thereof is administered at a dose of approximately 3 mg/kg and docetaxel is administered at a dose of approximately 75 mg/m² every three weeks.

In exemplary embodiments the anti-clusterin antibody or antigen binding fragment thereof is administered at a dose of approximately 3 mg/kg and docetaxel is administered at a dose of approximately 60 mg/m² every three weeks.

In exemplary embodiments the anti-clusterin antibody or antigen binding fragment thereof is administered at a dose of approximately 3 mg/kg and docetaxel is administered at a dose of approximately 50 mg/m² every two weeks.

In exemplary embodiment, the anti-clusterin antibody or antigen binding fragment thereof and docetaxel are administered once per week on same day.

In exemplary embodiment, the anti-clusterin antibody or antigen binding fragment thereof and docetaxel are administered once per week on same day.

In exemplary embodiment, the anti-clusterin antibody or antigen binding fragment thereof and docetaxel are administered every 2 weeks on same day.

In exemplary embodiment, the anti-clusterin antibody or antigen binding fragment thereof and docetaxel are administered every 3 weeks on same day.

In exemplary embodiment, the anti-clusterin antibody or antigen binding fragment thereof and docetaxel are administered every 4 weeks on same day.

In some embodiments, the method comprises administering a combination therapy comprising an anti-clusterin antibody or antigen binding fragment thereof at a dose of approximately 3 mg/kg to approximately 20 mg/kg once per week and docetaxel at a dose of approximately 50 mg/m² to 100 mg/m²once every two to three weeks.

In exemplary embodiments, the anti-clusterin antibody or antigen binding fragment thereof is administered at a dose of approximately 12 mg/kg once weekly and docetaxel is administered at a dose of approximately 75 mg/m² once every three weeks.

In exemplary embodiments the anti-clusterin antibody or antigen binding fragment thereof is administered at a dose of approximately 12 mg/kg once weekly and docetaxel is administered at a dose of approximately 60 mg/m² once every three weeks.

In exemplary embodiments the anti-clusterin antibody or antigen binding fragment thereof is administered at a dose of approximately 12 mg/kg once weekly and docetaxel is administered at a dose of approximately 50 mg/m² once every three weeks.

In exemplary embodiments the anti-clusterin antibody or antigen binding fragment thereof is administered at a dose of approximately 9 mg/kg once weekly and docetaxel is administered at a dose of approximately 75 mg/m² once every three weeks.

In exemplary embodiments the anti-clusterin antibody or antigen binding fragment thereof is administered at a dose of approximately 9 mg/kg once weekly and docetaxel is administered at a dose of approximately 60 mg/m² once every three weeks.

In exemplary embodiments the anti-clusterin antibody or antigen binding fragment thereof is administered at a dose of approximately 9 mg/kg once weekly and docetaxel is administered at a dose of approximately 50 mg/m² once every three weeks.

In exemplary embodiments the anti-clusterin antibody or antigen binding fragment thereof is administered at a dose of approximately 6 mg/kg once weekly and docetaxel is administered at a dose of approximately 75 mg/m² once every three weeks.

In exemplary embodiments the anti-clusterin antibody or antigen binding fragment thereof is administered at a dose of approximately 6 mg/kg once weekly and docetaxel is administered at a dose of approximately 60 mg/m² once every three weeks.

In exemplary embodiments the anti-clusterin antibody or antigen binding fragment thereof is administered at a dose of approximately 6 mg/kg once weekly and docetaxel is administered at a dose of approximately 50 mg/m² once every three weeks.

In exemplary embodiments the anti-clusterin antibody or antigen binding fragment thereof is administered at a dose of approximately 3 mg/kg once weekly and docetaxel is administered at a dose of approximately 75 mg/m² once every three weeks.

In exemplary embodiments the anti-clusterin antibody or antigen binding fragment thereof is administered at a dose of approximately 3 mg/kg once weekly and docetaxel is administered at a dose of approximately 60 mg/m² once every three weeks.

In exemplary embodiments the anti-clusterin antibody or antigen binding fragment thereof is administered at a dose of approximately 3 mg/kg once weekly and docetaxel is administered at a dose of approximately 50 mg/m² once every three weeks.

In some embodiments, the anti-clusterin antibody or antigen binding fragment thereof and docetaxel may be administered on same day. However, it is possible that they be administered on a different day.

In some embodiments, the anti-clusterin antibody or antigen binding fragment thereof and docetaxel may be administered on same day and separately.

The anti-clusterin antibody or antigen binding fragment thereof and docetaxel may be administered sequentially.

In some embodiments, the anti-clusterin antibody or antigen binding fragment thereof and/or docetaxel may be administered by infusion.

In some embodiments, the anti-clusterin antibody or antigen binding fragment thereof and docetaxel are both administered over the entire course of the treatment period.

In some embodiments, the anti-clusterin antibody or antigen binding fragment thereof and docetaxel are both administered at each cycle of treatment.

The anti-clusterin antibody or antigen binding fragment thereof and docetaxel are both generally administered at each treatment cycle. However, it is possible that one or more doses of the anti-clusterin antibody or antigen binding fragment thereof and/or docetaxel is missed without negatively impacting the treatment. It is also possible that one or more additional doses of the anti-clusterin antibody or antigen binding fragment thereof and/or docetaxel is administered without negatively impacting the treatment.

In accordance with the present disclosure, a subject may receive at least one treatment cycle.

In accordance with the present disclosure, a subject may receive at least two treatment cycles.

In accordance with the present disclosure, a subject may receive at least three treatment cycles.

In accordance with the present disclosure, a subject may receive at least four treatment cycles.

In accordance with the present disclosure, a subject may receive four or more treatment cycles.

In accordance with the present disclosure, a subject may receive at least five treatment cycles.

In accordance with the present disclosure, a subject may receive at least six treatment cycles.

In accordance with the present disclosure, a subject may receive at least seven treatment cycles.

In accordance with the present disclosure, a subject may receive at least eight treatment cycles.

In accordance with the present disclosure, a subject may receive at least nine treatment cycles

In accordance with the present disclosure, a subject may receive at least ten treatment cycles.

In accordance with the present disclosure, a subject may receive at least eleven treatment cycles.

In accordance with the present disclosure, a subject may receive at least twelve treatment cycles.

In accordance with the present disclosure, a subject may receive at least thirteen treatment cycles.

In accordance with the present disclosure, a subject may receive at least fourteen treatment cycles.

In accordance with the present disclosure, a subject may receive at least fifteen treatment cycles.

In accordance with the present disclosure, a subject may receive at least sixteen treatment cycles.

In accordance with the present disclosure, a subject may receive at least seventeen treatment cycles.

In accordance with the present disclosure, a subject may receive at least eighteen treatment cycles.

In accordance with the present disclosure, a subject may receive at least nineteen treatment cycles.

In accordance with the present disclosure, a subject may receive at least twenty treatment cycles.

In accordance with the present disclosure, a subject may receive more than twenty treatment cycles.

In accordance with the present disclosure, a subject may receive at least one treatment cycle comprising administration of the anti-clusterin antibody or antigen binding fragment thereof once per week and administration of docetaxel once every three weeks.

In accordance with the present disclosure, a subject may receive at least two treatment cycles each comprising administration of the anti-clusterin antibody or antigen binding fragment thereof once per week and administration of docetaxel once every three weeks.

In accordance with the present disclosure, a subject may receive at least three treatment cycles each comprising administration of the anti-clusterin antibody or antigen binding fragment thereof once per week and administration of docetaxel once every three weeks.

In accordance with the present disclosure, a subject may receive at least four treatment cycles each comprising administration of the anti-clusterin antibody or antigen binding fragment thereof once per week and administration of docetaxel once every three weeks.

In accordance with the present disclosure, a subject may receive four or more treatment cycles each comprising administration of the anti-clusterin antibody or antigen binding fragment thereof once per week and administration of docetaxel once every three weeks.

In accordance with the present disclosure, a subject may receive at least five treatment cycles each comprising administration of the anti-clusterin antibody or antigen binding fragment thereof once per week and administration of docetaxel once every three weeks.

In accordance with the present disclosure, a subject may receive at least six treatment cycles each comprising administration of the anti-clusterin antibody or antigen binding fragment thereof once per week and administration of docetaxel once every three weeks.

In accordance with the present disclosure, a subject may receive at least seven treatment each cycles comprising administration of the anti-clusterin antibody or antigen binding fragment thereof once per week and administration of docetaxel once every three weeks.

In accordance with the present disclosure, a subject may receive at least eight treatment cycles each comprising administration of the anti-clusterin antibody or antigen binding fragment thereof once per week and administration of docetaxel once every three weeks.

In accordance with the present disclosure, a subject may receive at least nine treatment cycles each comprising administration of the anti-clusterin antibody or antigen binding fragment thereof once per week and administration of docetaxel once every three weeks.

In accordance with the present disclosure, a subject may receive at least ten treatment cycles each comprising administration of the anti-clusterin antibody or antigen binding fragment thereof once per week and administration of docetaxel once every three weeks.

In accordance with the present disclosure, a subject may receive at least eleven treatment cycles each comprising administration of the anti-clusterin antibody or antigen binding fragment thereof once per week and administration of docetaxel once every three weeks.

In accordance with the present disclosure, a subject may receive at least twelve treatment cycles each comprising administration of the anti-clusterin antibody or antigen binding fragment thereof once per week and administration of docetaxel once every three weeks.

In accordance with the present disclosure, a subject may receive more than twelve treatment cycles each comprising administration of the anti-clusterin antibody or antigen binding fragment thereof once per week and administration of docetaxel once every three weeks.

In accordance with the present disclosure, the treatment cycles are consecutive.

In accordance with the present disclosure, all treatment cycles are consecutive. In accordance with the present disclosure, the treatment cycles are uninterrupted. In accordance with the present disclosure, all treatment cycles are uninterrupted.

The method or use may also involve interrupting treatment (single agent or combination therapy) for a period of time (e.g., for one week, two weeks, three weeks, four weeks, five weeks, six weeks, seven weeks, eight weeks, nine weeks, ten weeks, at least ten weeks, one month, two months, three months, four months, five months, six months, seven months, eight months, nine months, ten months, eleven months, at least six months, at least ten months, at least one year, once cycle, two cycles, three cycles, four cycles, five cycles, six cycles, seven cycles, eight cycles, nine cycles, ten cycles, at least ten cycles). Treatment may be reinitiated afterward.

In some embodiments, at least one treatment cycle is interrupted.

In other embodiments, more than one treatment cycles are interrupted.

In some embodiments the interruption lasts from one day to one week. In some embodiments the interruption lasts from one day to two weeks. In some embodiments the interruption lasts from one day to three weeks. In some embodiments the interruption lasts from one day to one month. In some embodiments the interruption lasts from one day to two months. In some embodiments the interruption lasts from one day to three months. In some embodiments the interruption lasts from one day to four months. In some embodiments the interruption lasts from one day to five months. In some embodiments the interruption lasts from one day to six months. In some embodiments the interruption lasts from one day to more than six months.

In some embodiments, the method particularly involves administering an anti-clusterin antibody or antigen binding fragment thereof comprises a light chain variable region comprising CDRs having the amino acid sequence set forth in SEQ ID NO:1 (CDRL1), in SEQ ID NO:2 (CDRL2) and in SEQ ID NO: 3 (CDRL3) and a heavy chain variable region comprising CDRs having the amino acid sequence set forth in SEQ ID NO:4 (CDRH1), SEQ ID NO: 5 (CDRH2) and SEQ ID NO:6 (CDRH3).

In other embodiments, the method particularly involves administering an anti-clusterin antibody or antigen binding fragment thereof comprises a light chain variable region comprising CDRs having the amino acid sequence set forth in SEQ ID NO:1 (CDRL1), in

SEQ ID NO:2 (CDRL2) and in SEQ ID NO: 3 (CDRL3) and a heavy chain variable region comprising CDRs having the amino acid sequence set forth in SEQ ID NO:7 (CDRH1), SEQ ID NO: 8 (CDRH2) and SEQ ID NO:9 (CDRH3).

In yet other embodiments, the method particularly involves administering an anti-clusterin antibody or antigen binding fragment thereof comprises:
a. a light chain variable region having an amino acid sequence having at least 80% identity with the amino acid sequence set forth in SEQ ID NO:12 and a heavy chain variable region having an amino acid sequence at least 80% identity with the amino acid sequence set forth in SEQ ID NO:13 or;
b. a light chain having an amino acid sequence having at least 80% identity with the amino acid sequence set forth in SEQ ID NO: 14 and a heavy chain having an amino acid sequence having at least 80% identity with the amino acid sequence set forth in SEQ ID NO: 15.

In other embodiments, the method particularly involves administering an anti-clusterin antibody or antigen binding fragment thereof comprises a light chain variable region having the amino acid sequence set forth in SEQ ID NO:12 and a heavy chain variable region having the amino acid sequence set forth in SEQ ID NO: 13.

In further embodiments, the method particularly involves administering anti-clusterin antibody or antigen binding fragment thereof comprises a light chain having the amino acid sequence set forth in SEQ ID NO:14 and a heavy chain having the amino acid sequence set forth in SEQ ID NO:15.

In some embodiments, the anti-clusterin antibody or antigen binding fragment thereof is administered by infusion over approximately a 1-hour time frame.

In some embodiments, docetaxel is administered by infusion over approximately a 1-hour time frame.

In some embodiments, the anti-clusterin antibody or antigen binding fragment thereof is administered by infusion over approximately a 1-hour time frame and docetaxel is subsequently administered by infusion on same day over approximately a 1-hour time frame.

In some embodiments, docetaxel is administered by infusion over approximately a 1-hour time frame and the anti-clusterin antibody or antigen binding fragment thereof is subsequently administered by infusion on same day over approximately a 1-hour time frame.

In accordance with the present disclosure, the combination therapy or medicament may be used for subjects having non-small cell lung cancer such as metastatic NSCLC or stage III to IV NSCLC.

In accordance with the present disclosure, the anti-clusterin antibody or antigen binding fragment thereof and docetaxel are both administered essentially over the entire course of the treatment period.

In accordance with the present disclosure, the combination therapy may be used in NSCLC patients that progressed after treatment with an immune checkpoint antibody either alone or in combination with chemotherapy.

Also, in accordance with the present disclosure, the combination therapy may be used in NSCLC patients with no evidence of PD-L1 expression (e.g., tumor proportion score (TPS)<1%) or with low expression of PD-L1 (e.g., tumor proportion score (TPS)≤15%, i.e., including <5%, ≤1%, <1% and 0%).

In accordance with the present disclosure, the combination therapy may be used in NSCLC that is not eligible for or would unlikely benefit from treatment comprising an immune checkpoint antibody.

The present disclosure relates to a combination therapy for use in a method of treating NSCLC such as metastatic NSCLC by promoting infiltration of immune cells in one or more lesions.

Accordingly, the present disclosure relates to a combination therapy for use in a method of treating NSCLC such as metastatic NSCLC by promoting infiltration of immune cells in one or more lesions that has low expression of PD-L1 or no evidence of PD-L1 expression.

### Exemplary Embodiments of Anti-Clusterin Antibodies or Antigen Binding Fragments Thereof

In some embodiments, the anti-clusterin antibody or antigen binding fragment thereof of the present disclosure is capable of inhibiting epithelial to mesenchymal transition.

In some embodiments, the anti-clusterin antibody or antigen binding fragment thereof of the present disclosure is capable of binding to amino acids 421 and 443 or to an epitope comprised within amino acids 421 and 443 of a C-terminal portion of a β-subunit of human clusterin (SEQ ID NO: 41 see PCT/CA2006/001505 published under No. WO2007/030930 and international application No. PCT/CA2010/0001882 published under No. WO2011/063523 the entire content of which is incorporated herein by reference). Such anti-clusterin antibodies or antigen binding fragments thereof are exemplified in Table 3 or comprise the CDRs, variable regions or full chains exemplified in Table 3. It is to be noted that in contrast with WO2011/063523 which describes a combination of an anti-clusterin antibody and a taxane, the present application provides evidence that the use of a combination therapy, particular doses or dosages, treatment regimen and/or schedule of administration described herein can induce tumor regression or stabilization of tumor growth in NSCLC patients.

In some embodiments, the anti-clusterin antibody or antigen binding fragment thereof is an antibody or antigen binding fragment thereof that is capable of competing with an anti-clusterin antibody or antigen binding fragment thereof of the present disclosure for the binding of clusterin (e.g., secreted clusterin (sCLU) or tumor-associated sCLU (TA-sCLU)) or for binding to a polypeptide comprising the amino acid sequence set forth in SEQ ID NO: 18.

In some embodiments, the CDRs are identified using methods known to a person skilled in the art and which are reviewed in Antibody Engineering Vol. 2, Chapter 3 by Andrew C. R. Martin, the entire content of which is incorporated herein by reference.

In particular embodiments, all CDRs are identified using the Kabat definition which is the most commonly used definition (Wu and Kabat, 1970).

In particular embodiments, all CDRs are identified using the contact definition (MacCallum et al., 1996) which is likely to be the most useful for people wishing to perform mutagenesis to modify the affinity of an antibody since these are residues which take part in interactions with antigen.

In accordance with the present disclosure, the anti-clusterin antibody or antigen binding fragment thereof comprises the amino acid sequence of CDRs, light chain and heavy chain variable regions or light chain and heavy chain set forth in Table 3.

In particular embodiments, the anti-clusterin antibody or antigen binding fragment thereof comprises a light chain variable region comprising the complementarity determining regions (CDRs) of the light chain variable region set forth in SEQ ID NO:12 and a heavy chain variable region comprising the CDRs of the heavy chain variable region set forth in SEQ ID NO:13.

In some exemplary embodiments, the anti-clusterin antibody or antigen binding fragment thereof comprises a light chain variable region comprising a CDRL1 having the amino acid sequence set forth in SEQ ID NO:1, a CDRL2 having the amino acid sequence set forth in SEQ ID NO:2, a CDRL3 having the amino acid sequence set forth in SEQ ID NO:3.

In some exemplary embodiments, the anti-clusterin antibody or antigen binding fragment thereof comprises a heavy chain variable region comprising a CDRH1 having the amino acid sequence set forth in SEQ ID NO:4, a CDRH2 having the amino acid sequence set forth in SEQ ID NO:5, a CDRH3 having the amino acid sequence set forth in SEQ ID NO: 6.

In some exemplary embodiments, the anti-clusterin antibody or antigen binding fragment thereof comprises a heavy chain variable region comprising a CDRH1 having the amino acid sequence set forth in SEQ ID NO:7, a CDRH2 having the amino acid sequence set forth in SEQ ID NO:8, a CDRH3 having the amino acid sequence set forth in SEQ ID NO: 9.

In some embodiments, the anti-clusterin antibody or antigen binding fragment thereof comprises a light chain variable region comprising a CDRL1 having the amino acid sequence set forth in SEQ ID NO:1, a CDRL2 having the amino acid sequence set forth in SEQ ID NO: 2, a CDRL3 having the amino acid sequence set forth in SEQ ID NO:3 and a heavy chain variable region comprising a CDRH1 having the amino acid sequence set forth in SEQ ID NO: 4, a CDRH2 having the amino acid sequence set forth in SEQ ID NO:5, a CDRH3 having the amino acid sequence set forth in SEQ ID NO:6.

In some embodiments, the anti-clusterin antibody or antigen binding fragment thereof comprises a light chain variable region comprising a CDRL1 having the amino acid sequence set forth in SEQ ID NO:1, a CDRL2 having the amino acid sequence set forth in SEQ ID NO: 2, a CDRL3 having the amino acid sequence set forth in SEQ ID NO:3 and a heavy chain variable region comprising a CDRH1 having the amino acid sequence set forth in SEQ ID NO: 7, a CDRH2 having the amino acid sequence set forth in SEQ ID NO:8, a CDRH3 having the amino acid sequence set forth in SEQ ID NO:9.

In some embodiments, the anti-clusterin antibody or antigen binding fragment thereof comprises a light chain variable region having an amino acid sequence having at least 80% identity with the amino acid sequence set forth in SEQ ID NO:10 and a heavy chain variable region having an amino acid sequence at least 80% identity with the amino acid sequence set forth in SEQ ID NO:11.

In some embodiments, the anti-clusterin antibody or antigen binding fragment thereof comprises a light chain variable region having an amino acid sequence having at least 90% identity with the amino acid sequence set forth in SEQ ID NO:10 and a heavy chain variable region having an amino acid sequence at least 90% identity with the amino acid sequence set forth in SEQ ID NO:11.

In some embodiments, the anti-clusterin antibody or antigen binding fragment thereof comprises a light chain variable region having an amino acid sequence identical to the amino acid sequence set forth in SEQ ID NO:10 and a heavy chain variable region having an amino acid sequence identical to the amino acid sequence set forth in SEQ ID NO:11.

In some embodiments, the anti-clusterin antibody or antigen binding fragment thereof is capable of competing with an antibody comprising a light chain variable region having the amino acid sequence set forth in SEQ ID NO:10 and a heavy chain variable region having the amino acid sequence set forth in SEQ ID NO:11 for the binding of clusterin (e.g., secreted clusterin (sCLU) or tumor-associated sCLU (TA-sCLU)) or for binding to a polypeptide comprising the amino acid sequence set forth in SEQ ID NO:18.

In accordance with the present disclosure, the anti-clusterin antibody or antigen binding fragment thereof comprises a light chain variable region having an amino acid sequence having at least 80%, at least 85%, at least 90%, at least 95%, at least 99% identity with the amino acid sequence set forth in SEQ ID NO:12 or is identical to or comprises the amino acid sequence set forth in SEQ ID NO:12 and a heavy chain variable region having an amino acid sequence at least 80%, at least 85%, at least 90%, at least 95%, at least 99% identity with the amino acid sequence set forth in SEQ ID NO:13 or is identical to or comprises the amino acid sequence set forth in SEQ ID NO:13.

In accordance with the present disclosure, the anti-clusterin antibody or antigen binding fragment thereof comprises a light chain having an amino acid sequence having at least 80%, at least 85%, at least 90%, at least 95%, at least 99% identity with the amino acid sequence set forth in SEQ ID NO:14 or is identical to or comprises the amino acid sequence set forth in SEQ ID NO:14 and a heavy chain having an amino acid sequence having at least 80%, at least 85%, at least 90%, at least 95%, at least 99% identity with the amino acid sequence set forth in SEQ ID NO:15 or is identical to or comprises the amino acid sequence set forth in SEQ ID NO:15.

In some embodiments, the anti-clusterin antibody or antigen binding fragment thereof comprises a light chain variable region having an amino acid sequence having at least 80% identity with the amino acid sequence set forth in SEQ ID NO: 12 and a heavy chain variable region having an amino acid sequence at least 80% identity with the amino acid sequence set forth in SEQ ID NO:13.

In some embodiments, the anti-clusterin antibody or antigen binding fragment thereof comprises a light chain variable region having an amino acid sequence having at least 90% identity with the amino acid sequence set forth in SEQ ID NO: 12 and a heavy chain variable region having an amino acid sequence at least 90% identity with the amino acid sequence set forth in SEQ ID NO:13.

In some embodiments, the anti-clusterin antibody or antigen binding fragment thereof comprises a light chain variable region having an amino acid sequence identical to the amino acid sequence set forth in SEQ ID NO:12 and a heavy chain variable region having an amino acid sequence identical to the amino acid sequence set forth in SEQ ID NO:13.

In accordance with the present disclosure, the antibody or antigen binding fragment thereof is capable of competing with an antibody comprising a light chain variable region having an amino acid sequence set forth in SEQ ID NO: 12 and a heavy chain variable region having an amino acid sequence set forth in SEQ ID NO: 13 for the binding of clusterin (e.g., secreted clusterin (sCLU) or tumor-associated sCLU (TA-sCLU)) or for binding to a polypeptide comprising the amino acid sequence set forth in SEQ ID NO:18.

In some embodiments, the anti-clusterin antibody or antigen binding fragment thereof comprises a light chain having an amino acid sequence having at least 80% identity with the amino acid sequence set forth in SEQ ID NO:14 and a heavy chain having an amino acid sequence having at least 80% identity with the amino acid sequence set forth in SEQ ID NO: 15.

In some embodiments, the anti-clusterin antibody or antigen binding fragment thereof comprises a light chain having an amino acid sequence having at least 90% identity with the amino acid sequence set forth in SEQ ID NO:14 and a heavy chain having an amino acid sequence having at least 90% identity with the amino acid sequence set forth in SEQ ID NO: 15.

In some embodiments, the anti-clusterin antibody or antigen binding fragment thereof comprises a light chain having an amino acid sequence identical the amino acid sequence set forth in SEQ ID NO:14 and a heavy chain having an amino acid sequence identical to the amino acid sequence set forth in SEQ ID NO:15.

In some embodiments, the anti-clusterin antibody or antigen binding fragment thereof is capable of competing with an antibody comprising a light chain having the amino acid sequence set forth in SEQ ID NO:14 and a heavy chain having the amino acid sequence set forth in SEQ ID NO: 15 for the binding of clusterin (e.g., secreted clusterin (sCLU) or tumor-associated sCLU (TA-sCLU)) or for binding to a polypeptide comprising the amino acid sequence set forth in SEQ ID NO: 18.

In yet other particular embodiments, the anti-clusterin antibody or antigen binding fragment thereof comprises the CDRs, variable regions or full chains amino acid sequence of the antibody or antigen binding fragment thereof listed in Table 3.

In some embodiments, the anti-clusterin antibody or antigen binding fragment thereof is humanized 16B5.

In some embodiments, the anti-clusterin antibody or antigen binding fragment thereof is a humanized 16B5 biosimilar.

The amino acid sequence of murine 16B5, humanized 16B5, murine 21B12 and humanized 21B12 is disclosed in international application No. PCT/CA2010/001882 filed on Nov. 24, 2010 and published on Jun. 3, 2011 under No. WO2011/063523, the entire content of which is incorporated herein by reference.

In yet further particular embodiments, the anti-clusterin antibody or antigen binding fragment thereof may be able to compete with one or more of the antibodies or antigen binding fragments thereof listed in Table 3.

Exemplary embodiments of antibodies or antigen binding fragment thereof that competes with AB-16B5 includes for example and without limitations, antibodies identified as 16B5, 21B12, 20E11, 11E2 and 16C11 disclosed in international application No. PCT/CA2006/001505 filed on Sep. 13, 2006 and published on Mar. 22, 2007 under no. WO2007/030930 the entire content of which is incorporated herein by reference. Such anti-clusterin antibodies or antigen binding fragments thereof are exemplified in Table 3.

### Exemplary Embodiments of Subject

The combination therapy disclosed herein is administered to a subject in need.

The combination therapy disclosed herein is generally administered to a human subject.

In some aspects and embodiments of the present disclosure, the subject has cancer.

In other aspects and embodiments of the present disclosure, the subject is a subject having cancer and having a functional immune system.

In yet other aspects and embodiments of the present disclosure, the subject is a subject having cancer and adequate organ and immune function.

In some embodiments, the subject has non-small cell lung cancer.

In some embodiments, the subject has metastatic NSCLC.

In some embodiments, the subject has stage III to IV NSCLC.

In other embodiments, the subject has or is selected for having NSCLC that progressed after treatment comprising an immune checkpoint antibody.

In other embodiments, the subject has or is selected for having NSCLC that progressed after chemotherapy.

In some embodiments, the subject has received prior treatment with chemotherapy.

In other embodiments, the subject has or is selected for having NSCLC that is not eligible for or would unlikely benefit from treatment that comprises immune checkpoint antibody.

A subject may be characterized as being not eligible for treatment with a PD-1 or PD-L1 immune checkpoint inhibitor based on objective criteria such as for example, prior failure of treatment comprising a PD-1 or PD-L1 immune checkpoint inhibitor, level of expression of PD-1 or PD-L1, level of immune cell infiltration in the tumor environment and the like.

A subject may be characterized as being unlikely to benefit from treatment with a PD-1 or PD-L1 immune checkpoint inhibitor based on subjective criteria such as, physician assessment of risks and benefits, overall clinical condition of the subject, and the like.

In yet other embodiments, the subject has or is selected for having NSCLC that has failed prior treatment with an immune checkpoint antibody and a platinum-containing doublet treatment.

In some embodiments, the subject has or is selected for having NSCLC that has failed prior treatment comprising an anti-PD-1 immune checkpoint antibody.

In some embodiments, the subject has or is selected for having NSCLC that has failed prior treatment comprising an PD-L1 immune checkpoint antibody.

In some embodiments, the subject has or is selected for having NSCLC that has failed prior treatment comprising an anti-PD-1 or PD-L1 immune checkpoint antibody.

In some embodiments, the subject has or is selected for having NSCLC that has failed prior treatment with an anti-PD-1 immune checkpoint antibody and a platinum-containing doublet treatment.

In some embodiments, the subject has or is selected for having NSCLC that has failed prior treatment with an anti-PD-L1 immune checkpoint antibody and a platinum-containing doublet treatment.

In additional embodiments, the subject has or is selected for having NSCLC that has failed prior treatment with an anti-PD-1 or PD-L1 immune checkpoint antibody and a platinum-containing doublet treatment.

In accordance with the present disclosure, the doublet treatment may be provided simultaneously.

In accordance with the present disclosure, the doublet treatment may be provided sequentially.

In some embodiments, the subject has or is selected for having metastatic NSCLC that has failed prior treatment with an anti-PD-1 or PD-L1 immune checkpoint antibody and a platinum-containing doublet treatment.

In some embodiments, the subject has or is selected for having NSCLC that has failed prior treatment with immune checkpoint antibody selected from ipilimumab, nivolumab, pembrolizumab, cemiplimab, atezolizumab, avelumab, or durvalumab alone or in combination with platinum-based chemotherapy.

In some embodiments, the subject has or is selected for having NSCLC that has failed prior treatment with pembrolizumab alone or in combination with platinum-based chemotherapy.

In other embodiments, the subject has or is selected for having metastatic NSCLC that progressed after treatment comprising an immune checkpoint antibody.

In other embodiments, the subject has or is selected for having metastatic NSCLC that progressed after chemotherapy.

In yet other embodiments, the subject has or is selected for having metastatic NSCLC that has failed prior treatment with immune checkpoint antibody and chemotherapy treatment.

In other embodiments, the subject has or is selected for having metastatic NSCLC that is not eligible for or would unlikely benefit from treatment that comprises immune checkpoint antibody.

In yet other embodiments, the subject has or is selected for having metastatic NSCLC that has failed prior treatment with immune checkpoint antibody and a platinum-containing doublet treatment.

In some embodiments, the subject has or is selected for having metastatic NSCLC that has failed prior treatment comprising an anti-PD-1 immune checkpoint antibody.

In some embodiments, the subject has or is selected for having metastatic NSCLC that has failed prior treatment comprising an PD-L1 immune checkpoint antibody.

In some embodiments, the subject has or is selected for having metastatic NSCLC that has failed prior treatment comprising an anti-PD-1 or PD-L1 immune checkpoint antibody.

In some embodiments, the subject has or is selected for having metastatic NSCLC that has failed prior treatment with an anti-PD-1 immune checkpoint antibody and a platinum-containing doublet treatment.

In some embodiments, the subject has or is selected for having metastatic NSCLC that has failed prior treatment with an anti-PD-L1 immune checkpoint antibody and a platinum-containing doublet treatment.

In additional embodiments, the subject has or is selected for having metastatic NSCLC that has failed prior treatment with an anti-PD-1 or PD-L1 immune checkpoint antibody and a platinum-containing doublet treatment.

In some embodiments, the subject has or is selected for having metastatic NSCLC that has failed prior treatment with an anti-PD-1 or PD-L1 immune checkpoint antibody and a platinum-containing doublet treatment.

In some embodiments, the subject has or is selected for having metastatic NSCLC that has failed prior treatment with immune checkpoint antibody selected from ipilimumab, nivolumab, pembrolizumab, cemiplimab, atezolizumab, avelumab, or durvalumab alone or in combination with platinum-based chemotherapy.

In some embodiments, the subject has or is selected for having metastatic NSCLC that has failed prior treatment with pembrolizumab alone or in combination with platinum-based chemotherapy.

In addition to having or being selected for having NSCLC or a metastatic NSCLC, the subject may have or may be selected for having one or more characteristics described herein and/or exemplified below.

In some embodiments, the subject has or is selected for having one or more lesions with a PD-L1 tumor proportion score of ≥50%.

In some embodiments, the subject has or is selected for having one or more lesions with a PD-L1 tumor proportion score of <50%.

In some embodiments, the subject has or is selected for having one or more lesions with a PD-L1 tumor proportion score of between 1% to 49%.

In some embodiments, the subject has or is selected for having one or more lesions with a PD-L1 tumor proportion score of ≤15%.

In some embodiments, the subject has or is selected for having one or more lesions with a PD-L1 tumor proportion score of <5%.

In some embodiments, the subject has or is selected for having one or more lesions with a PD-L1 tumor proportion score of ≤1%.

In some embodiments, the subject has or is selected for having one or more lesions with a PD-L1 tumor proportion score of <1%.

In some embodiments, the subject has or is selected for having one or more lesions with a PD-L1 tumor proportion score of 0%.

In some embodiments, the tumor proportion score is determined at baseline.

In some embodiments, the tumor proportion score is determined prior to administration of the combination therapy.

In some embodiments, the tumor proportion score is determined after first-line therapy.

In some embodiments, the tumor proportion score is determined at the onset of the combination therapy.

In some embodiments, the PD-L1 tumor proportion score is determined by a scientist or pathologist.

In some embodiments, the PD-L1 tumor proportion score is determined in accordance with drug regulator's guidance (e.g., FDA, EMA and the like).

In some embodiments, the subject has or is selected for having one or more lesions that are immunologically cold.

In some embodiments, the subject has or is selected for having one or more lesions with poor infiltration of immune cells. The level of immune cell infiltration may be determined by a trained pathologist. The level of immune cell infiltration may be determined by computer-based quantification of tumor biopsy imaging.

In some embodiments, the subject has or is selected for having one or more lesions having an EMT signature or showing signs of an EMT signature. The EMT signature may be determined by a trained pathologist. The EMT signature may be determined by computer-based quantification of tumor or tumor biopsy imaging.

In accordance with the present disclosure, the subject is a subject having non-small cell lung cancer such as, for example, metastatic non-small cell lung cancer and is selected based on the following non-exhaustive and non-limitative criteria:
a) The subject has failed prior therapy with an immune checkpoint antibody either alone or in combination with chemotherapy;
b) The subject has a one or more lesions with poor infiltration of immune cells;
c) The subject has one or more lesions having an EMT signature or showing signs of an EMT signature;
d) The subject has one or more criteria listed in any one of a) to c).

Alternatively, in accordance with the present disclosure, the subject is a subject having non-small cell lung cancer such as, for example metastatic non-small cell lung cancer and is selected based on the following non-exhaustive and non-limitative criteria;
a) The subject has not received prior therapy with an anti-PD-L1 antibody either alone or in combination with platinum-based chemotherapy;
b) The subject has one or more lesions with a PD-L1 tumor proportion score of ≤ 15%;
c) The subject has one or more lesions with poor infiltration of immune cells;
d) The subject has one or more lesions having an EMT signature or showing signs of an EMT signature or;
e) The subject has one or more criteria listed in any one of a) to d).

A subject may thus be selected based on one criterion or a combination of criteria disclosed herein.

In exemplary embodiments, the subject has a functional immune system and/or an adequate organ and immune function.

In exemplary embodiments, the subject has or is selected for having NSCLC, such as metastatic non-small cell lung cancer, and has not received prior therapy with an anti-PD-L1 antibody.

In other exemplary embodiments, the subject has or is selected for having NSCLC, such as metastatic non-small cell lung cancer, and one or more lesions with a PD-L1 tumor proportion score of ≤15%.

In another exemplary embodiment, the subject has or is selected for having NSCLC, such as metastatic non-small cell lung cancer, and a PD-L1 tumor proportion score of ≤15% and has not received prior therapy with an anti-PD-L1 antibody.

In other exemplary embodiments, the subject has or is selected for having NSCLC, such as metastatic non-small cell lung cancer, and a PD-L1 tumor proportion score of ≤15% and for having one or more lesions with poor infiltration of immune cells.

In other exemplary embodiments, the subject has or is selected for having NSCLC, such as metastatic non-small cell lung cancer, and a PD-L1 tumor proportion score of <1%.

In another exemplary embodiment, the subject has or is selected for having NSCLC, such as metastatic non-small cell lung cancer, and a PD-L1 tumor proportion score of <1% and has not received prior therapy with an anti-PD-L1 antibody.

In other exemplary embodiments, the subject has or is selected for having NSCLC, such as metastatic non-small cell lung cancer, and a PD-L1 tumor proportion score of <1% and for having one or more lesions with poor infiltration of immune cells.

In some instances, the subject has or is selected for having NSCLC, such as metastatic non-small cell lung cancer, and that has failed prior therapy with an immune checkpoint antibody either alone or in combination with chemotherapy.

In some instances, the subject has or is selected for having NSCLC, such as metastatic non-small cell lung cancer, that has failed first-line, second-line, third-line or subsequent line therapy.

In some instances, the combination therapy is administered as a first-line therapy in a subject having or selected for having NSCLC, such as metastatic non-small cell lung cancer, and a PD-L1 tumor proportion score of ≤15%.

In some instances, the combination therapy is administered as a first-line therapy in a subject having or is selected for having NSCLC, such as metastatic non-small cell lung cancer, and a PD-L1 tumor proportion score of <1%.

In some instances, the combination therapy is administered as a second-line therapy in a subject having NSCLC, such as metastatic non-small cell lung cancer, and that has failed prior therapy with an immune checkpoint antibody either alone or in combination with chemotherapy.

In some instances, the combination therapy is administered as a second-line therapy in a subject having NSCLC, such as metastatic non-small cell lung cancer, and that has failed prior therapy with an immune checkpoint antibody either alone or in combination with chemotherapy.

In accordance with the present disclosure, the subject in need has a tumor that expresses or secrete clusterin.

In accordance with the present disclosure, the subject in need is a subject that is not immunosuppressed or has not received an immunosuppressive medication within 14, 13, 12, 11, 10, 9, 8, 7, 6, 5, 4, 3, 2 days or 1 day prior to treatment. In some embodiments, the subject in need may have received corticosteroids prior to treatment.

In some embodiments, the subject is or selected for being immunocompetent.

In some embodiments, the subject has or is selected for not being immunosuppressed. In some embodiments, the subject has or is selected for not having received an immunosuppressive medication within 7 days prior to treatment.

In accordance with the present disclosure, the subject in need is a subject that has not received prior treatment with docetaxel.

It is to be understood herein that treatment failure is often associated with a disease progression or undesired side effects of treatment. As such, in some embodiments subjects that has failed prior treatment includes subjects having progressive disease. In other embodiments subjects that has failed prior treatment includes subjects having undesired side effects associated with the prior treatment.

In some embodiments, the subject has not received prior treatment with docetaxel.

### PD-L1 Expression and Immune Cells Infiltration

PD-L1 expression may be determined using methods known to a person skilled in the art.

In an exemplary embodiment, the PD-L1 tumor proportion score (TPS) is assessed with the 22C3 antibody (Agilent Technologies, Carpinteria, CA, code SK006).

In another exemplary embodiment, the PD-L1 TPS is assessed with the 28-8 antibody (Agilent Technologies, Carpinteria, CA, code SK005; Abcam, Toronto, Canada, Cat. No. Ab205921).

In yet another exemplary embodiment, PD-L1 TPS is assessed with the SP263 antibody Roche Diagnostic Ref. 740-4907.

In a further exemplary embodiment, the PD-L1 TPS is assessed with the SP142 antibody (Abcam, Toronto, Canada, Cat. No. Ab228462).

In a further exemplary embodiment, the PD-L1 TPS is assessed with the 73-10 antibody (clone MKP1A07310; Merck KGaA, Darmstadt, Germany; Abcam, Toronto, Canada, Cat. No. Ab228415).

The PD-L1 expression may be assessed using an anti-PD-L1 antibody suitable for the type of carcinoma that the subject has.

The PD-L1 expression is usually assessed in tumor cells. However, the PD-L1 expression may be assessed both in tumor cells and in tumor-infiltrating immune cells.

In some instances, the PD-L1 expression is assessed by immunohistochemistry.

In other instances, the PD-L1 expression is assessed by fluorescence assays, such as for example, by quantitative fluorescence or fluorescence in-situ hybridization.

In some embodiments, the assay is an assay approved by at least one regulatory body. In accordance with the present disclosure, the assay is a United States Food and Drug Administration (FDA)-approved diagnostic test (Dx).

In accordance with the present disclosure, the assay is an FDA-approved test for research (Rx).

In an exemplary embodiment, the assay is based on PD-L1 IHC 22C3 pharmDx (Agilent Technologies, Carpinteria, CA, code SK006).

In another exemplary embodiment, the assay is based on PD-L1 IHC 28-8 pharmDx ((Agilent Technologies, Carpinteria, CA, code SK005)).

In yet another exemplary embodiment, the assay is based on Ventana PD-L1 (SP263) (Roche Diagnostic Ref. 740-4907).

In some aspects and embodiments, the immunohistochemistry assay is performed in accordance with manufacturer's instructions.

In some embodiments, the assay uses Autostainer Link 48 immunohistochemistry platform (Dako).

In some embodiments, the assay uses the Omnis immunohistochemistry platform (Dako).

In some embodiments, the assay is performed with the Bond-III immunohistochemistry platform (Leica).

In some embodiments, the assay uses the BenchMark ULTRA immunohistochemistry platform (Ventana).

The assay results are interpreted using the manufacturer's interpretation manual.

Exemplary assays are described in Munari, E., et al., 2021, Marchetti, A. et al., 2017, Yoshikawa, K. et al., 2021, the entire content of all of which is incorporated herein by reference.

In some embodiments, the absence or presence of immune cells in the tumor microenvironment may be confirmed by tumor biopsy.

In other embodiments, the absence or presence of immune cells in the tumor microenvironment may be confirmed by in vivo imaging (e.g., magnetic resonance imaging, e.g., see Jiang X. et al., 2020).

A tumor may be characterized as “immunologically cold” when the tumor microenvironment is not sufficiently infiltrated by immune cells (especially by lymphocytes) or when the tumor microenvironment is not inflamed. In contrast, a tumor may be characterized as “immunologically warm” or “immunologically hot” when infiltration of immune cells (especially by lymphocytes) in the tumor microenvironment is observed or when the tumor shows sign of inflammation.

Generally, a pathologist, a technologist, a trained scientist, or trained technician equipped with proper reagents and/or apparatus may be able to determine the absence or presence of immune cells in the tumor microenvironment and may thus be able to evaluate whether a tumor is “immunologically cold”, “immunologically warm” or “immunologically hot”.

### Exemplary Embodiments of Kits

The present disclosure provides in some aspects and embodiments, a kit comprising one or more containers comprising at least one dose of an anti-clusterin antibody or antigen binding fragment thereof, one or more containers comprising at least one dose of a taxane for use in combination therapy and a package insert comprising instructions for treating a subject in need.

The present disclosure provides in other aspects and embodiments, a kit comprising one or more containers comprising at least one dose of an anti-clusterin antibody or antigen binding fragment thereof, one or more containers comprising at least one dose of docetaxel for use in combination therapy and a package insert comprising instructions for treating a subject in need.

In accordance with the present disclosure, the anti-clusterin antibody or antigen binding fragment thereof and docetaxel are provided in separate containers.

In accordance with the present disclosure, the antibody or antigen binding fragment thereof is as described herein.

In some embodiments, the package insert states that the combination therapy is intended for treatment of a subject as described herein.

In some embodiments, the package insert states that the combination therapy is intended for treatment of a subject having NSCLC.

In some embodiments, the package insert states that the combination therapy is intended for treatment of a subject having metastatic NSCLC such as for example, stage III-IV NSCLC.

In some embodiments, the package insert states that the combination therapy is intended for treatment of a subject having NSCLC that progressed after treatment comprising an immune checkpoint antibody.

In some embodiments, the package insert states that the combination therapy is intended for treatment of a subject having NSCLC that has failed prior treatment with immune checkpoint antibody and a platinum-containing doublet treatment.

In some embodiments, the package insert states that the combination therapy is intended for treatment of a subject having metastatic NSCLC that progressed after a first-line treatment comprising an immune checkpoint antibody.

In some embodiments, the package insert states that the combination therapy is intended for treatment of a subject having metastatic NSCLC that has failed prior treatment comprising an immune checkpoint antibody and a platinum-containing doublet treatment.

In addition to the embodiments described and provided in this disclosure, the following non-limiting embodiments are particularly contemplated.

1. A method of treating a subject having lung cancer, the method comprising administering the combination therapy disclosed herein.

2. A method of treating a subject having non-small cell lung cancer, the method comprising administering the combination therapy disclosed herein.

3. A method of treating a subject having non-small cell lung cancer, the method comprising administering the combination therapy disclosed herein so as to obtain and/or maintain a clinical benefit.

4. The method of any one of the preceding embodiments, wherein the combination therapy comprises an anti-clusterin antibody or antigen binding fragment thereof and a taxane.

5. A method of procuring a clinical benefit to a subject having non-small cell lung cancer, the method comprising administering the combination therapy disclosed herein.

6. A method of procuring a clinical benefit to a subject having non-small cell lung cancer, in a method of treatment comprising administering a combination therapy for at least two cycles of treatment, each cycle of treatment comprising administration of the anti-clusterin antibody at a dose of approximately 3 mg/kg to approximately 20 mg/kg once per week and administration of a taxane at a dose of approximately 60 mg/m² and 100 mg/m² once every three weeks, wherein the anti-clusterin antibody comprises a light chain variable region comprising the complementarity determining regions (CDRs) of the light chain variable region set forth in SEQ ID NO:12 and a heavy chain variable region comprising the CDRs of the heavy chain variable region set forth in SEQ ID NO: 13.

7. A method of procuring a clinical benefit to a subject having non-small cell lung cancer, in a method of treatment comprising administering a combination therapy for at least two cycles of treatment, each cycle of treatment comprising administration of the anti-clusterin antibody at a dose of approximately 12 mg/kg once per week and administration of docetaxel at a dose of approximately 75 mg/m² once every three weeks, wherein the anti-clusterin antibody comprises a light chain variable region comprising the complementarity determining regions (CDRs) of the light chain variable region set forth in SEQ ID NO: 12 and a heavy chain variable region comprising the CDRs of the heavy chain variable region set forth in SEQ ID NO:13.

8. A method of treating a subject having non-small cell lung cancer, the method comprising administering a combination therapy comprising an anti-clusterin antibody or antigen binding fragment thereof and a taxane, wherein the anti-clusterin antibody or antigen binding fragment thereof comprises a light chain variable region comprising the complementarity determining regions (CDRs) of the light chain variable region set forth in SEQ ID NO:12 and a heavy chain variable region comprising the CDRs of the heavy chain variable region set forth in SEQ ID NO:13.

9. A method of treating a subject having non-small cell lung cancer, the method comprising administering a combination therapy comprising an anti-clusterin antibody or antigen binding fragment thereof and a taxane so as to obtain a clinical benefit, wherein the anti-clusterin antibody or antigen binding fragment thereof comprises a light chain variable region comprising the complementarity determining regions (CDRs) of the light chain variable region set forth in SEQ ID NO:12 and a heavy chain variable region comprising the CDRs of the heavy chain variable region set forth in SEQ ID NO: 13.

10. The method of any one of the preceding embodiments, wherein the taxane is docetaxel or paclitaxel.

11. A method of treating non-small cell lung cancer in a subject in need thereof, comprising administering a combination therapy for at least two cycles of treatment, each cycle of treatment comprising administration of the anti-clusterin antibody at a dose of approximately 3 mg/kg to approximately 20 mg/kg once per week, once every two weeks, or once every three weeks and administration of docetaxel at a dose of approximately 50 mg/m² to approximately 100 mg/m² once every two to three weeks, wherein the anti-clusterin antibody comprises a light chain variable region comprising the complementarity determining regions (CDRs) of the light chain variable region set forth in SEQ ID NO: 12 and a heavy chain variable region comprising the CDRs of the heavy chain variable region set forth in SEQ ID NO:13.

12. The method of any one of the preceding embodiments, wherein the anti-clusterin antibody or antigen binding fragment thereof is administered at a dose of between approximately 3 mg/kg to approximately 20 mg/kg.

13. The method of any one of the preceding embodiments, wherein the anti-clusterin antibody or antigen binding fragment thereof is administered at a dose of between approximately 6 mg/kg to approximately 20 mg/kg.

14. The method of any one of the preceding embodiments, wherein the anti-clusterin antibody or antigen binding fragment thereof is administered at a dose of between approximately 6 mg/kg to approximately 15 mg/kg.

15. The method of any one of the preceding embodiments, wherein the anti-clusterin antibody or antigen binding fragment thereof is administered at a dose of between approximately 6 mg/kg to approximately 14 mg/kg.

16. The method of any one of the preceding embodiments, wherein the anti-clusterin antibody or antigen binding fragment thereof is administered at a dose of between approximately 6 mg/kg to approximately 13 mg/kg.

17. The method of any one of the preceding embodiments, wherein the anti-clusterin antibody or antigen binding fragment thereof is administered at a dose of between approximately 6 mg/kg to approximately 12 mg/kg.

18. The method of any one of the preceding embodiments, wherein the anti-clusterin antibody or antigen binding fragment thereof is administered at a dose of between approximately 7 mg/kg to approximately 12 mg/kg.

19. The method of any one of the preceding embodiments, wherein the anti-clusterin antibody or antigen binding fragment thereof is administered at a dose of between approximately 8 mg/kg to approximately 12 mg/kg.

20. The method of any one of the preceding embodiments, wherein the anti-clusterin antibody or antigen binding fragment thereof is administered at a dose of between approximately 9 mg/kg to approximately 12 mg/kg.

21. The method of any one of the preceding embodiments, wherein the anti-clusterin antibody or antigen binding fragment thereof is administered at a dose of between approximately 10 mg/kg to approximately 12 mg/kg.

22. The method of any one of the preceding embodiments, wherein the anti-clusterin antibody or antigen binding fragment thereof is administered at a dose of between approximately 11 mg/kg to approximately 12 mg/kg.

23. The method of any one of the preceding embodiments, wherein the anti-clusterin antibody or antigen binding fragment thereof is administered at a dose of approximately 3 mg/kg.

24 The method of any one of the preceding embodiments, wherein the anti-clusterin antibody or antigen binding fragment thereof is administered at a dose of approximately 6 mg/kg.

25. The method of any one of the preceding embodiments, wherein the anti-clusterin antibody or antigen binding fragment thereof is administered at a dose of approximately 9 mg/kg.

26. The method of any one of the preceding embodiments, wherein the anti-clusterin antibody or antigen binding fragment thereof is administered at a dose of approximately 12 mg/kg.

27. The method of any one of the preceding embodiments, wherein the taxane is docetaxel.

28. The method of any one of the preceding embodiments, wherein the taxane is docetaxel and is administered at a dose of between approximately 50 mg/m² to approximately 100 mg/m².

29 The method of any one of the preceding embodiments, wherein the taxane is docetaxel and is administered at a dose of between approximately 60 mg/m² to approximately 100 mg/m².

30. The method of any one of the preceding embodiments, wherein docetaxel is administered at a dose of between approximately 60 mg/m² to approximately 95 mg/m².

31. The method of any one of the preceding embodiments, wherein docetaxel is administered at a dose of between approximately 60 mg/m² to approximately 90 mg/m².

32 The method of any one of the preceding embodiments, wherein docetaxel is administered at a dose of between approximately 60 mg/m² to approximately 85 mg/m².

33. The method of any one of the preceding embodiments, wherein docetaxel is administered at a dose of between approximately 60 mg/m² to approximately 80 mg/m².

34. The method of any one of the preceding embodiments, wherein docetaxel is administered at a dose of between approximately 50 mg/m² to approximately 75 mg/m².

The method of any one of the preceding embodiments, wherein docetaxel is administered at a dose of between approximately 60 mg/m² to approximately 75 mg/m².

36 The method of any one of the preceding embodiments, wherein docetaxel is administered at a dose of between approximately 75 mg/m² to approximately 80 mg/m².

37. The method of any one of the preceding embodiments, wherein the taxane is docetaxel and is administered at a dose of approximately 50 mg/m².

38. The method of any one of the preceding embodiments, wherein the taxane is docetaxel and is administered at a dose of approximately 60 mg/m².

39. The method of any one of the preceding embodiments, wherein the taxane is docetaxel and is administered at a dose of approximately 75 mg/m².

40. The method of any one of the preceding embodiments, wherein the taxane is docetaxel and is administered at a dose of approximately 100 mg/m².

41. The method of any one of the preceding embodiments, wherein the anti-clusterin antibody or antigen binding fragment thereof is administered once weekly (once per week).

42. The method of any one of the preceding embodiments, wherein the anti-clusterin antibody or antigen binding fragment thereof is administered once every two weeks. 43. The method of any one of the preceding embodiments, wherein the anti-clusterin antibody or antigen binding fragment thereof is administered once every three weeks.

44. The method of any one of the preceding embodiments, wherein the anti-clusterin antibody or antigen binding fragment thereof is administered once every four weeks.

45. The method of any one of the preceding embodiments, wherein docetaxel is administered once per week.

46. The method of any one of the preceding embodiments, wherein docetaxel is administered once every two weeks.

47. The method of any one of the preceding embodiments, wherein docetaxel is administered once every three weeks.

48. The method of any one of the preceding claims, wherein docetaxel is administered once every four weeks.

49. A method of treating a subject having non-small cell lung cancer, the method comprising administering a combination therapy comprising an anti-clusterin antibody or antigen binding fragment thereof at a dose of approximately 12 mg/kg once per week, once every two weeks, once every three weeks, once every four weeks and docetaxel at a dose of approximately 50 mg/m² to approximately 75 mg/m² once every two to three weeks so as to obtain a clinical benefit, wherein the anti-clusterin antibody or antigen binding fragment thereof comprises a light chain variable region comprising the complementarity determining regions (CDRs) of the light chain variable region set forth in SEQ ID NO:12 and a heavy chain variable region comprising the CDRs of the heavy chain variable region set forth in SEQ ID NO:13.

50. A method of treating non-small cell lung cancer in a subject in need thereof, comprising administering a combination therapy for at least two cycles of treatment, each cycle of treatment comprising administration of the anti-clusterin antibody at a dose of approximately 12 mg/kg once per week, once every two weeks, once every three weeks or once every four weeks and administration of docetaxel at a dose of approximately 50 mg/m² to approximately 75 mg/m² once every two to three weeks, wherein the anti-clusterin antibody comprises a light chain variable region comprising the complementarity determining regions (CDRs) of the light chain variable region set forth in SEQ ID NO:12 and a heavy chain variable region comprising the CDRs of the heavy chain variable region set forth in SEQ ID NO:13.

51. The method of any one of the preceding embodiments, wherein the subject is as described herein.

52. The method of any one of the preceding embodiments, wherein the anti-clusterin antibody or antigen binding fragment thereof and docetaxel are administered on same day.

53. The method of any one of the preceding embodiments, wherein the anti-clusterin antibody or antigen binding fragment thereof and docetaxel are administered on different days.

54. The method of any one of the preceding embodiments, wherein the subject has or is selected for having NSCLC that has failed prior treatment with an immune checkpoint antibody.

55. The method of any one of the preceding embodiments, wherein the subject has or is selected for having NSCLC that has failed prior treatment with chemotherapy.

56. The method of any one of the preceding embodiments, wherein the subject has or is selected for having NSCLC that has failed prior treatment with an immune checkpoint antibody with or without chemotherapy.

57. The method of any one of the preceding embodiments, wherein the subject has or is selected for having NSCLC that has failed prior treatment comprising an anti-PD-1 immune checkpoint antibody.

58. The method of any one of the preceding embodiments, wherein the subject has or is selected for having NSCLC that has failed prior treatment comprising a programmed death ligand 1 (PD-L1) immune checkpoint antibody.

59. The method of any one of the preceding embodiments, wherein the subject has or is selected for having NSCLC that has failed prior treatment comprising an anti-PD-1 or PD-L1 immune checkpoint antibody.

60. The method of any one of the preceding embodiments, wherein the subject has or is selected for having NSCLC that has failed prior treatment with an immune checkpoint antibody and chemotherapy.

61. The method of any one of the preceding embodiments, wherein the subject has or is selected for having NSCLC that has failed prior treatment with an anti-PD-1 immune checkpoint antibody and a platinum-containing doublet treatment.

62. The method of any one of the preceding embodiments, wherein the subject has or is selected for having NSCLC that has failed prior treatment with an anti-PD-L1 immune checkpoint antibody and a platinum-containing doublet treatment.

63. The method of any one of the preceding embodiments, wherein the subject has or is selected for having NSCLC that has failed prior treatment with an anti-PD-1 or PD-L1 immune checkpoint antibody and a platinum-containing doublet treatment.

64. The method of any one of the preceding embodiments, wherein the immune checkpoint antibody is selected from ipilimumab, nivolumab, pembrolizumab, cemiplimab, atezolizumab, avelumab, or durvalumab.

65. The method of any one of the preceding embodiments, wherein the immune checkpoint antibody is pembrolizumab.

66. The method of any one of the preceding embodiments, wherein the subject has or is selected for having one or more lesions with a PD-L1 tumor proportion score (TPS) of ≥ 50%.

67. The method of any one of the preceding embodiments, wherein the subject has or is selected for having one or more lesions with a PD-L1 tumor proportion score (TPS) of <50%.

68. The method of any one of the preceding embodiments, wherein the subject has or is selected for having one or more lesions with a PD-L1 tumor proportion score (TPS) of between 1% to 49%.

69. The method of any one of the preceding embodiments, wherein the subject has or is selected for having one or more lesions with a PD-L1 tumor proportion score of ≤15%.

70. The method of any one of the preceding embodiments, wherein the subject has or is selected for having one or more lesions with a PD-L1 tumor proportion score of <5%.

71. The method of any one of the preceding embodiments, wherein the subject has or is selected for having one or more lesions with a PD-L1 tumor proportion score of ≤1%.

72. The method of any one of the preceding embodiments, wherein the subject has or is selected for having one or more lesions with a PD-L1 tumor proportion score of <1%.

73. The method of any one of the preceding embodiments, wherein the subject has or is selected for having one or more lesions with no evidence of PD-L1 expression.

74. The method of any one of the preceding embodiments, wherein the subject has or is selected for having NSCLC that is not eligible for treatment comprising an immune checkpoint antibody.

75. The method of any one of the preceding embodiments, wherein the subject has or is selected for having NSCLC and would unlikely benefit from treatment comprising an immune checkpoint antibody.

76. The method of any one of the preceding embodiments, wherein the tumor proportion score is determined prior to administration of the combination therapy.

77. The method of any one of the preceding embodiments, wherein the subject that has or is selected for having NSCLC characterized as having a KRAS mutation.

78. The method of any one of the preceding embodiments, wherein the subject has or is selected for having NSCLC comprising one or more lesions carrying a KRAS mutation.

79. The method of any one of the preceding embodiments, wherein the subject has or is selected for having a tumor that has poor infiltration of immune cells.

80 The method of any one of the preceding embodiments, wherein the subject has or is selected for having one or more lesions having an EMT signature.

81. The method of any one of the preceding embodiments, wherein the subject has or is selected for having one or more lesions showing signs of an EMT signature.

82. The method of any one of the preceding embodiments, wherein the combination therapy is administered as a cycle of treatment including administration of the anti-clusterin antibody or antigen binding fragment thereof on days 1, 8 and 15 and administration of docetaxel on day 1.

83 The method of any one of the preceding embodiments, wherein one cycle of treatment lasts 1 week.

84. The method of any one of the preceding embodiments, wherein one cycle of treatment lasts 2 weeks.

85. The method of any one of the preceding embodiments, wherein one cycle of treatment lasts 3 weeks.

86. The method of any one of the preceding embodiments, wherein one cycle of treatment lasts 4 weeks.

87. The method of any one of the preceding embodiments, wherein one cycle of treatment lasts more than 4 weeks.

88. The method of any one of the preceding embodiments, wherein the anti-clusterin antibody or antigen binding fragment thereof is administered by IV infusion.

89. The method of any one of the preceding embodiments, wherein docetaxel is administered by IV infusion.

90. The method of any one of the preceding embodiments, wherein the clinical benefit is regression of a lesion.

91. The method of any one of the preceding embodiments, wherein the clinical benefit is a decrease in the size of a lesion.

92. The method of any one of the preceding embodiments, wherein the clinical benefit is a stabilization in the size of a lesion.

93. The method of any one of the preceding embodiments, wherein the clinical benefit is a reduction in the number of tumor cells in a lesion.

94. The method of any one of the preceding embodiments, wherein the decrease in size is at least 5%.

95. The method of any one of the preceding embodiments, wherein the decrease in size is at least 10%.

96. The method of any one of the preceding embodiments, wherein the decrease in size is at least 15%.

97. The method of any one of the preceding embodiments, wherein the decrease in size is at least 20%.

98 The method of any one of the preceding embodiments, wherein the decrease in size is at least 25%.

99 The method of any one of the preceding embodiments, wherein the decrease in size is at least 30%.

100. The method of any one of the preceding embodiments, wherein the decrease in size is at least 35%.

101. The method of any one of the preceding embodiments, wherein the decrease in size is at least 40%.

102. The method of any one of the preceding embodiments, wherein the decrease in size is at least 45%.

103. The method of any one of the preceding embodiments, wherein the decrease in size is at least 50%, 104. The method of any one of the preceding embodiments, wherein the clinical benefit is an increase of less than 20% in the size of a lesion. 105. The method of any one of the preceding embodiments, wherein the clinical benefit is defined as per RECIST 1.1 guidelines.

106. The method of any one of the preceding embodiments, wherein the clinical benefit is improved quality of life.

107. The method of any one of the preceding embodiments, wherein the method procures a clinical benefit for at least 6 weeks after first determination of response to treatment.

108. The method of any one of the preceding embodiments, wherein the method procures a clinical benefit for at least 12 weeks after first determination of response to treatment.

109. The method of any one of the preceding embodiments, wherein the method procures a clinical benefit for at least 18 weeks after first determination of response to treatment.

110. The method of any any one of the preceding embodiments, wherein the method procures a clinical benefit for at least 24 weeks after first determination of response to treatment.

111. The method of any one of the preceding embodiments, wherein the method procures a clinical benefit for at least 30 weeks after first determination of response to treatment.

112. The method of any one of the preceding embodiments, wherein the method procures a clinical benefit for at least 36 weeks after first determination of response to treatment.

113. The method of any one of the preceding embodiments, wherein the method procures a clinical benefit for at least 42 weeks after first determination of response to treatment.

114. The method of any one of the preceding embodiments, wherein the clinical benefit is selected from complete response (CR) as per RECIST 1.1 guidelines, partial response (PR) as per RECIST 1.1 guidelines or stable disease (SD) as per RECIST 1.1 guidelines.

115. The method of any one of the preceding embodiments, wherein the clinical benefit is partial response (PR) as per RECIST 1.1 guidelines.

116. The method of any one of the preceding embodiments, wherein the clinical benefit is stable disease (SD) as per RECIST 1.1 guidelines.

117. The method of any one of the preceding embodiments, wherein the size of the lesion is assessed in accordance with RECIST 1.1 guidelines.

118. The method of any one of the preceding embodiments, wherein the size of the lesion is measured by CT scan.

119. The method of any one of the preceding embodiments, wherein the clinical benefit is a reduction in or an elimination of metabolic activity in a lesion.

120. The method of any one of the preceding embodiments, wherein the metabolic activity is assessed by PET scan (e.g., (18) F-fluorodeoxyglucose positron emission tomographic (FDG-PET).

121. The method of any one of the preceding embodiments, wherein the clinical benefit is assessed with tumor biomarkers.

122. The method of any one of the preceding embodiments, wherein the clinical benefit is an improvement in a symptom associated with said cancer.

123. The method of any one of the preceding embodiments, wherein the clinical benefit is observed after two cycles of treatment.

124. The method of any one of the preceding embodiments, wherein the clinical benefit is observed for at least 12 weeks of treatment.

125. The method of any one of the preceding embodiments, wherein the clinical benefit is observed for at least 18 weeks of treatment.

126. The method of any one of the preceding embodiments, wherein the clinical benefit is observed for at least 24 weeks of treatment.

127. The method of any one of the preceding embodiments, wherein the clinical benefit is observed for at least 30 weeks of treatment.

128. The method of any one of the preceding embodiments, wherein the clinical benefit is observed for at least 36 weeks of treatment.

129. The method of any one of the preceding embodiments, wherein the clinical benefit is observed for at least 42 weeks of treatment.

130. The method of any one of the preceding embodiments, wherein the dose of docetaxel is reduced or treatment with docetaxel is interrupted upon docetaxel-associated toxicity.

131. The method of any one of the preceding embodiments, wherein the method comprises administering at least one initial cycle of treatment and at least one subsequent cycle of treatment, wherein the initial cycle of treatment and subsequent cycle of treatment are identical or different.

132. The method of any one of the preceding embodiments, wherein each of the initial cycle of treatment and subsequent cycle of treatment comprises at least one dose of the anti-clusterin antibody or antigen binding fragment thereof and at least one dose of docetaxel.

133. The method of any one of the preceding embodiments, wherein the initial cycle of treatment and/or subsequent cycle of treatment each independently comprises administering the anti-clusterin antibody or antigen binding fragment thereof at a dose of approximately 3 mg/kg to approximately 20 mg/kg once per week, once every two weeks, or once every three weeks and administration of docetaxel at a dose of approximately 50 mg/m² to 100 mg/m² once every two weeks or once every three weeks.

134. The method of any one of the preceding embodiments, wherein the method comprises administering docetaxel at a dose of approximately 75 mg/m² once every three weeks and administering the anti-clusterin antibody or antigen binding fragment thereof once per week, once every two weeks or once every three weeks.

135. The method of any one of the preceding embodiments, wherein the method comprises administering the anti-clusterin antibody or antigen binding fragment thereof at a dose of approximately 12 mg/kg once per week and administering docetaxel at a dose of approximately 75 mg/m² once every three weeks.

136. The method of any one of the preceding embodiments, wherein the method comprises administering the anti-clusterin antibody or antigen binding fragment thereof at a dose of approximately 12 mg/kg once every three weeks and administering docetaxel at a dose of approximately 75 mg/m² once every three weeks.

137. The method of any one of the preceding embodiments, wherein the method comprises administering:
a. at least one initial cycle of treatment comprising administering the anti-clusterin antibody or antigen binding fragment thereof at a dose of approximately 12 mg/kg once per week and administration of docetaxel at a dose of approximately 75 mg/m² once every three weeks and;
b. at least one subsequent cycle of treatment comprising administering the anti-clusterin antibody or antigen binding fragment thereof at a dose of approximately 3 mg/kg to approximately 20 mg/kg once every three weeks and administration of docetaxel at a dose of approximately 75 mg/m² once every three weeks.

138. The method of any one of the preceding embodiments, wherein the method comprises administering:
a. at least one initial cycle of treatment comprising administering the anti-clusterin antibody or antigen binding fragment thereof at a dose of approximately 12 mg/kg once per week and administration of docetaxel at a dose of approximately 75 mg/m² once every three weeks and;
b. at least one subsequent cycle of treatment comprising administering the anti-clusterin antibody or antigen binding fragment thereof at a dose of approximately 12 mg/kg once every three weeks and administration of docetaxel at a dose of approximately 75 mg/m² once every three weeks.

139. The method of any one of the preceding embodiments, wherein the method comprises administering docetaxel at a dose of approximately 50 mg/m² once every two weeks and administering the anti-clusterin antibody or antigen binding fragment thereof once per week, once every two weeks or once every three weeks.

140. The method of any one of the preceding embodiments, wherein the method comprises administering the anti-clusterin antibody or antigen binding fragment thereof at a dose of approximately 12 mg/kg once per week and administering docetaxel at a dose of approximately 50 mg/m² once every two weeks.

141. The method of any one of the preceding embodiments, wherein the method comprises administering the anti-clusterin antibody or antigen binding fragment thereof at a dose of approximately 12 mg/kg once every two weeks and administering docetaxel at a dose of approximately 50 mg/m² once every two weeks.

142. The method of any one of the preceding embodiments, wherein the method comprises administering:
a) at least one initial cycle of treatment comprising administering the anti-clusterin antibody or antigen binding fragment thereof at a dose of approximately 12 mg/kg once per week and administration of docetaxel at a dose of approximately 50 mg/m² once every two weeks and;
b) at least one subsequent cycle of treatment comprising administering the anti-clusterin antibody or antigen binding fragment thereof at a dose of approximately 3 mg/kg to approximately 20 mg/kg once every two weeks and administration of docetaxel at a dose of approximately 50 mg/m² once every two weeks.

143. The method of any one of the preceding embodiments, wherein the method comprises administering:
a) at least one initial cycle of treatment comprising administering the anti-clusterin antibody or antigen binding fragment thereof at a dose of approximately 12 mg/kg once per week and administration of docetaxel at a dose of approximately 50 mg/m² once every two weeks and;
b) at least one subsequent cycle of treatment comprising administering the anti-clusterin antibody or antigen binding fragment thereof at a dose of approximately 12 mg/kg once every two weeks and administration of docetaxel at a dose of approximately 50 mg/m² once every two weeks.

144. The method of any one of the preceding embodiments, wherein the combination therapy is administered for two treatment cycles or more, each comprising administration of the anti-clusterin antibody or antigen binding fragment thereof once every two weeks and administration of docetaxel once every two weeks.

145. The method of any one of the preceding embodiments, wherein the combination therapy is administered for two treatment cycles or more, each comprising administration of the anti-clusterin antibody or antigen binding fragment thereof once every three weeks and administration of docetaxel once every three weeks. 146. The method of any one of the preceding embodiments, wherein the combination therapy is administered for at least 2 cycles. 147. The method of any one of the preceding embodiments, wherein the combination therapy is administered for at least 3 cycles.

148. The method of any one of the preceding embodiments, wherein the combination therapy is administered for at least 4 cycles.

149. The method of any one of the preceding embodiments, wherein the combination therapy is administered for at least 5 cycles.

150. The method of any one of the preceding embodiments, wherein the combination therapy is administered for at least 6 cycles.

151. The method of any one of the preceding embodiments, wherein the combination therapy is administered for at least 7 cycles.

152. The method of any one of the preceding embodiments, wherein the combination therapy is administered for at least 8 cycles.

153. The method of any one of the preceding embodiments, wherein the combination therapy is administered for at least 9 cycles.

154. The method of any one of the preceding embodiments, wherein the combination therapy is administered for at least 10 cycles.

155. The method of any one of the preceding embodiments, wherein the combination therapy is administered for at least 11 cycles.

156. The method of any one of the preceding embodiments, wherein the combination therapy is administered for more than 11 cycles.

157. The method of any one of the preceding embodiments, wherein the combination therapy is administered for at least 12 cycles.

158. The method of any one of the preceding embodiments, wherein the combination therapy is administered for at least 14 cycles.

159. The method of any one of the preceding embodiments, wherein the combination therapy is administered for at least 16 cycles.

160. The method of any one of the preceding embodiments, wherein the combination therapy is administered for at least 18 cycles.

161. The method of any one of the preceding embodiments, wherein the combination therapy is administered for at least 20 cycles.

162. The method of any one of the preceding embodiments, wherein the combination therapy is administered for at least 22 cycles.

163. The method of any one of the preceding embodiments, wherein the combination therapy is administered for at least one to at least five initial cycles of treatment.

164. The method of any one of the preceding embodiments, wherein the combination therapy is administered for two or more subsequent cycles of treatment.

165. The method of any one of the preceding embodiments, wherein the combination therapy is administered for at least one treatment cycle comprising administration of the anti-clusterin antibody or antigen binding fragment thereof once per week and administration of docetaxel every three weeks.

166. The method of any one of the preceding embodiments, wherein the combination therapy is administered for at least two treatment cycles each comprising administration of the anti-clusterin antibody or antigen binding fragment thereof once per week and administration of docetaxel once every three weeks.

167. The method of any one of the preceding embodiments, wherein the combination therapy is administered for at least three treatment cycles each comprising administration of the anti-clusterin antibody or antigen binding fragment thereof once per week and administration of docetaxel once every three weeks.

168. The method of any one of the preceding embodiments, wherein the combination therapy is administered for at least four treatment cycles each comprising administration of the anti-clusterin antibody or antigen binding fragment thereof once per week and administration of docetaxel once every three weeks.

169. The method of any one of the preceding embodiments, wherein the combination therapy is administered for at least four or more treatment cycles each comprising administration of the anti-clusterin antibody or antigen binding fragment thereof once per week and administration of docetaxel once every three weeks.

170. The method of any one of the preceding embodiments, wherein the combination therapy is administered for at least five treatment cycles each comprising administration of the anti-clusterin antibody or antigen binding fragment thereof once per week and administration of docetaxel once every three weeks.

171. The method of any one of the preceding embodiments, wherein the combination therapy is administered for at least six treatment cycles each comprising administration of the anti-clusterin antibody or antigen binding fragment thereof once per week and administration of docetaxel once every three weeks.

172. The method of any one of the preceding embodiments, wherein the combination therapy is administered for at least seven treatment cycles each comprising administration of the anti-clusterin antibody or antigen binding fragment thereof once per week and administration of docetaxel once every three weeks.

173. The method of any one of the preceding embodiments, wherein the combination therapy is administered for at least eight treatment cycles each comprising administration of the anti-clusterin antibody or antigen binding fragment thereof once per week and administration of docetaxel once every three weeks.

174. The method of any one of the preceding embodiments, wherein the combination therapy is administered for at least nine treatment cycles each comprising administration of the anti-clusterin antibody or antigen binding fragment thereof once per week and administration of docetaxel once every three weeks.

175. The method of any one of the preceding embodiments, wherein the combination therapy is administered for at least ten treatment cycles each comprising administration of the anti-clusterin antibody or antigen binding fragment thereof once per week and administration of docetaxel once every three weeks.

176. The method of any one of the preceding embodiments, wherein the combination therapy is administered for at least eleven treatment cycles each comprising administration of the anti-clusterin antibody or antigen binding fragment thereof once per week and administration of docetaxel once every three weeks.

177. The method of any one of the preceding embodiments, wherein the combination therapy is administered for at least twelve treatment cycles each comprising administration of the anti-clusterin antibody or antigen binding fragment thereof once per week and administration of docetaxel once every three weeks.

178. The method of any one of the preceding embodiments, wherein the combination therapy is administered for more than twelve treatment cycles each comprising administration of the anti-clusterin antibody or antigen binding fragment thereof once per week and administration of docetaxel once every three weeks.

179. The method of any one of the preceding embodiments, wherein the treatment cycles are consecutives.

180. The method of any one of the preceding embodiments, wherein administration of the combination therapy is administered as long as a clinical benefit is observed.

181. The method of any one of the preceding embodiments, wherein administration of the combination therapy is administered until tumor progression.

182. The method of any one of the preceding embodiments, wherein administration of the combination therapy is administered until unmanageable toxicity occurs.

183. The method of any one of the preceding embodiments, wherein administration of the anti-clusterin antibody or antigen binding fragment thereof is maintained after the appearance of clinical benefits.

184. The method of any one of the preceding embodiments, wherein the anti-clusterin antibody or antigen binding fragment thereof inhibits epithelial to mesenchymal transition.

185. The method of any one of the preceding embodiments, wherein the anti-clusterin antibody or antigen binding fragment thereof is capable of binding to a C-terminal portion of a β-subunit of human clusterin.

186. The method of any one of the preceding embodiments, wherein the anti-clusterin antibody or antigen binding fragment thereof is capable of binding to amino acids 421 and 443 or to an epitope comprised within amino acids 421 and 443 of a C-terminal portion of a β-subunit of human clusterin.

187. The method of any one of the preceding embodiments, wherein the anti-clusterin antibody or antigen binding fragment thereof comprises a light chain variable region comprising the complementarity determining regions (CDRs) of the light chain variable region set forth in SEQ ID NO: 12 and a heavy chain variable region comprising the CDRs of the heavy chain variable region set forth in SEQ ID NO:13.

188. The method of any one of the preceding embodiments, wherein the anti-clusterin antibody or antigen binding fragment thereof comprises a light chain variable region comprising CDRs having the amino acid sequence set forth in SEQ ID NO:1 (CDRL1), in SEQ ID NO:2 (CDRL2) and in SEQ ID NO: 3 (CDRL3) and a heavy chain variable region comprising CDRs having the amino acid sequence set forth in SEQ ID NO:4 (CDRH1), SEQ ID NO: 5 (CDRH2) and SEQ ID NO:6 (CDRH3).

189. The method of any one of the preceding embodiments, wherein the anti-clusterin antibody or antigen binding fragment thereof comprises a light chain variable region comprising CDRs having the amino acid sequence set forth in SEQ ID NO:1 (CDRL1), in SEQ ID NO:2 (CDRL2) and in SEQ ID NO: 3 (CDRL3) and a heavy chain variable region comprising CDRs having the amino acid sequence set forth in SEQ ID NO:7 (CDRH1), SEQ ID NO: 8 (CDRH2) and SEQ ID NO:9 (CDRH3).

190. The method of any one of the preceding embodiments, wherein the anti-clusterin antibody or antigen binding fragment thereof comprises:
a. a light chain variable region having an amino acid sequence having at least 80% identity with the amino acid sequence set forth in SEQ ID NO:12 and a heavy chain variable region having an amino acid sequence at least 80% identity with the amino acid sequence set forth in SEQ ID NO:13 or;
b. a light chain having an amino acid sequence having at least 80% identity with the amino acid sequence set forth in SEQ ID NO: 14 and a heavy chain having an amino acid sequence having at least 80% identity with the amino acid sequence set forth in SEQ ID NO: 15.

191. The method of any one of the preceding embodiments, wherein the anti-clusterin antibody or antigen binding fragment thereof comprises a light chain variable region having the amino acid sequence set forth in SEQ ID NO: 12 and a heavy chain variable region having the amino acid sequence set forth in SEQ ID NO:13.

192. The method of any one of the preceding embodiments, wherein the anti-clusterin antibody or antigen binding fragment thereof comprises a light chain having the amino acid sequence set forth in SEQ ID NO:14 and a heavy chain having the amino acid sequence set forth in SEQ ID NO: 15.

193. The method of any one of the preceding embodiments, wherein the subject is not immunosuppressed or has not received an immunosuppressive medication within 7 days prior to treatment.

194. The method of any one of the preceding embodiments, wherein the subject has not received prior treatment with docetaxel.

195. The method of any one of the preceding embodiments, wherein the subject does not receive concurrent anti-cancer treatment with the anti-clusterin antibody or antigen binding fragment thereof and/or docetaxel combination treatment.

196. The method of any one of the preceding embodiments, wherein the subject is a human subject.

197. The method of any one of the preceding embodiments, wherein the subject has a functional immune system.

198. The method of any one of the preceding embodiments, wherein the subject has an adequate organ and/or immune function.

199. The method of any one of the preceding embodiments, wherein the NSCLC is metastatic NSCLC.

200. The method of any one of the preceding embodiments, wherein the metastatic NSCLC is stage III NSCLC.

201. The method of any one of the preceding embodiments, wherein the metastatic NSCLC is stage IV NSCLC.

202. The method of any one of the preceding embodiments, wherein the metastatic

NSCLC is a stage III to IV NSCLC.

203. The method of any one of the preceding embodiments, wherein the subject having NSCLC is a subject having metastatic NSCLC.

204. The method of any one of the preceding embodiments, wherein the subject having metastatic NSCLC is a subject having stage III NSCLC.

205. The method of any one of the preceding embodiments, wherein the subject having metastatic NSCLC is a subject having stage IV NSCLC.

206. The method of any one of the preceding embodiments, wherein the subject has or is selected for having a stage III to IV NSCLC.

207. The method of any one of the preceding embodiments, wherein the method comprises a step of promoting infiltration of immune cells in the tumor microenvironment of one or more lesions.

208. The method of any one of the preceding embodiments, wherein the method promotes infiltration of immune cells in the tumor microenvironment of one or more lesions.

209. The method of any one of the preceding embodiments, wherein the method comprises administering immunotherapy subsequently to the combination therapy.

210. The method of any one of the preceding embodiments, wherein the lesion is a tumor metastasis.

211. The method of any one of the preceding embodiments, wherein the lesion is the primary tumor.

212. The method of any one of the preceding embodiments, wherein one or more lesions have a PD-L1 tumor proportion score (TPS) of ≤15%.

213. The method of any one of the preceding embodiments, wherein one or more lesions have a PD-L1 tumor proportion score (TPS) of <5%.

214. The method of any one of the preceding embodiments, wherein one or more lesions have a PD-L1 tumor proportion score (TPS) of ≤1%.

215. The method of any one of the preceding embodiments, wherein one or more lesions have a PD-L1 tumor proportion score (TPS) of <1%.

216. The method of any one of the preceding embodiments, wherein one or more lesions have a PD-L1 combined positive score (CPS) of <10%.

217. The method of any one of the preceding embodiments, wherein one or more lesions have a PD-L1 combined positive score (CPS) of <5%.

218. The method of any one of the preceding embodiments, wherein one or more lesions have a PD-L1 combined positive score (CPS) of <1%.

219. The method of any one of the preceding embodiments, wherein PD-L1 expression is undetectable or non-evaluable.

220. The method of any one of the preceding embodiments, wherein one or more lesions is characterized as being immunologically cold.

221. The method of any one of the preceding embodiments, wherein the combination therapy is provided as a first-line therapy.

222. The method of any one of the preceding embodiments, wherein the combination therapy is provided as a second-line therapy.

223. The method of any one of the preceding embodiments, wherein the combination therapy is provided as a subsequent therapy.

224. The method of any one of the preceding embodiments, wherein the subject is administered with a therapeutically effective combination therapy.

225. The method of any one of the preceding embodiments, wherein docetaxel is discontinued.

226. The method of any one of the preceding embodiments, wherein docetaxel is discontinued upon signs of toxicity and treatment with the anti-clusterin antibody or antigen binding fragment thereof is continued.

227. The method of any one of the preceding embodiments, wherein docetaxel is ceased for one or more cycles of treatment.

228. The method of any one of the preceding embodiments, wherein docetaxel is ceased for one or more cycles of treatment upon signs of toxicity and treatment with the anti-clusterin antibody or antigen binding fragment thereof is continued.

229. The method of any one of the preceding embodiments, wherein the anti-clusterin antibody or antigen binding fragment thereof is a conventional antibody.

230. A combination therapy for use in a method of any one of the preceding embodiments.

231. A combination therapy comprising an anti-clusterin antibody or antigen binding fragment thereof and a taxane.

232. A combination therapy comprising an anti-clusterin antibody or antigen binding fragment thereof and a taxane for treatment of lung cancer.

233. A therapeutically effective combination therapy comprising an anti-clusterin antibody or antigen binding fragment thereof and a taxane for use in the treatment of non-small cell lung cancer (NSCLC) in a subject in need thereof.

234. A combination therapy comprising an anti-clusterin antibody or antigen binding fragment thereof and a taxane for use in treatment of a subject having non-small cell lung cancer, wherein the anti-clusterin antibody or antigen binding fragment thereof comprises a light chain variable region comprising the complementarity determining regions (CDRs) of the light chain variable region set forth in SEQ ID NO:12 and a heavy chain variable region comprising the CDRs of the heavy chain variable region set forth in SEQ ID NO: 13.

235. A combination therapy comprising an anti-clusterin antibody or antigen binding fragment thereof and a taxane for use in procuring a clinical benefit to a subject having non-small cell lung cancer, wherein the anti-clusterin antibody or antigen binding fragment thereof comprises a light chain variable region comprising the complementarity determining regions (CDRs) of the light chain variable region set forth in SEQ ID NO: 12 and a heavy chain variable region comprising the CDRs of the heavy chain variable region set forth in SEQ ID NO:13.

236. The combination therapy of any one of the preceding embodiments, wherein the taxane is docetaxel or paclitaxel.

237. The combination therapy of any one of the preceding embodiments, wherein the anti-clusterin antibody or antigen binding fragment thereof is formulated for administration at a dose of between approximately 3 mg/kg to approximately 20 mg/kg.

238. The combination therapy of any one of the preceding embodiments, wherein the anti-clusterin antibody or antigen binding fragment thereof is formulated for administration at a dose of between approximately 6 mg/kg to approximately 20 mg/kg.

239. The combination therapy of any one of the preceding embodiments, wherein the anti-clusterin antibody or antigen binding fragment thereof is formulated for administration at a dose of between approximately 6 mg/kg to approximately 15 mg/kg.

240. The combination therapy of any one of the preceding embodiments, wherein the anti-clusterin antibody or antigen binding fragment thereof is formulated for administration at a dose of between approximately 6 mg/kg to approximately 14 mg/kg.

241. The combination therapy of any one of the preceding embodiments, wherein the anti-clusterin antibody or antigen binding fragment thereof is formulated for administration at a dose of between approximately 6 mg/kg to approximately 13 mg/kg.

242. The combination therapy of any one of the preceding embodiments, wherein the anti-clusterin antibody or antigen binding fragment thereof is formulated for administration at a dose of between approximately 6 mg/kg to approximately 12 mg/kg.

243. The combination therapy of any one of the preceding embodiments, wherein the anti-clusterin antibody or antigen binding fragment thereof is formulated for administration at a dose of between approximately 7 mg/kg to approximately 12 mg/kg.

244. The combination therapy of any one of the preceding embodiments, wherein the anti-clusterin antibody or antigen binding fragment thereof is formulated for administration at a dose of between approximately 8 mg/kg to approximately 12 mg/kg.

245. The combination therapy of any one of the preceding embodiments, wherein the anti-clusterin antibody or antigen binding fragment thereof is formulated for administration at a dose of between approximately 9 mg/kg to approximately 12 mg/kg.

246. The combination therapy of any one of the preceding embodiments, wherein the anti-clusterin antibody or antigen binding fragment thereof is formulated for administration at a dose of between approximately 10 mg/kg to approximately 12 mg/kg.

247. The combination therapy of any one of the preceding embodiments, wherein the anti-clusterin antibody or antigen binding fragment thereof is formulated for administration at a dose of between approximately 11 mg/kg to approximately 12 mg/kg.

248. The combination therapy of any one of the preceding embodiments, wherein the anti-clusterin antibody or antigen binding fragment thereof is formulated for administration at a dose of approximately 12 mg/kg.

249. The combination therapy of any one of the preceding embodiments, wherein the taxane is docetaxel.

250. The combination therapy of any one of the preceding embodiments, wherein the taxane is docetaxel and is formulated for administration at a dose of between approximately 50 mg/m² to approximately 100 mg/m².

251. The combination therapy of any one of the preceding embodiments, wherein the taxane is docetaxel and is formulated for administration at a dose of between approximately 60 mg/m² to approximately 100 mg/m².

252. The combination therapy of any one of the preceding embodiments, wherein docetaxel is formulated for administration at a dose of between approximately 60 mg/m² to approximately 95 mg/m²

253. The combination therapy of any one of the preceding embodiments, wherein docetaxel is formulated for administration at a dose of between approximately 60 mg/m² to approximately 90 mg/m².

254. The combination therapy of any one of the preceding embodiments, wherein docetaxel is formulated for administration at a dose of between approximately 60 mg/m² to approximately 85 mg/m².

255. The combination therapy of any one of the preceding embodiments, wherein docetaxel is formulated for administration at a dose of between approximately 60 mg/m² to approximately 80 mg/m².

256. The combination therapy of any one of the preceding embodiments, wherein docetaxel is formulated for administration at a dose of between approximately 50 mg/m² to approximately 75 mg/m².

257. The combination therapy of any one of the preceding embodiments, wherein docetaxel is formulated for administration at a dose of between approximately 60 mg/m² to approximately 75 mg/m².

258. The combination therapy of any one of the preceding embodiments, wherein docetaxel is formulated for administration at a dose of between approximately 75 mg/m² to approximately 80 mg/m².

259. The combination therapy of any one of the preceding embodiments, wherein docetaxel is formulated for administration at a dose of approximately 50 mg/m².

260. The combination therapy of any one of the preceding embodiments, wherein docetaxel is formulated for administration at a dose of approximately 75 mg/m².

261. A combination therapy comprising an anti-clusterin antibody or antigen binding fragment thereof and docetaxel for use in procuring a clinical benefit to a subject having non-small cell lung cancer, wherein the anti-clusterin antibody or antigen binding fragment thereof is formulated for administration at a dose of approximately 12 mg/kg and comprises a light chain variable region comprising the complementarity determining regions (CDRs) of the light chain variable region set forth in SEQ ID NO:12 and a heavy chain variable region comprising the CDRs of the heavy chain variable region set forth in SEQ ID NO: 13 and docetaxel is formulated for administration at a dose of approximately 50 mg/m² to approximately 75 mg/m².

262. A combination therapy for use in procuring a clinical benefit to a subject having non-small cell lung cancer, wherein the combination therapy is used for at least two cycles of treatment each cycle of treatment comprising administration of the anti-clusterin antibody at a dose of approximately 3 mg/kg to approximately 20 mg/kg once per week and administration of a taxane at a dose of approximately 60 mg/m² to 100 mg/m² once every three weeks, wherein the anti-clusterin antibody comprises a light chain variable region comprising the complementarity determining regions (CDRs) of the light chain variable region set forth in SEQ ID NO:12 and a heavy chain variable region comprising the CDRs of the heavy chain variable region set forth in SEQ ID NO:13.

263. A combination therapy for use in procuring a clinical benefit to a subject having non-small cell lung cancer, wherein the combination therapy is used for at least two cycles of treatment each cycle of treatment comprising administration of the anti-clusterin antibody at a dose of approximately 12 mg/kg once per week and administration of docetaxel at a dose of approximately 50 mg/m² to approximately 75 mg/m² once every three weeks, wherein the anti-clusterin antibody comprises a light chain variable region comprising the complementarity determining regions (CDRs) of the light chain variable region set forth in SEQ ID NO:12 and a heavy chain variable region comprising the CDRs of the heavy chain variable region set forth in SEQ ID NO:13.

264. The combination therapy of any one of the preceding embodiments, wherein the combination therapy is for use a subject as described herein.

265. The combination therapy of any one of the preceding embodiments, wherein the combination therapy is for use in a subject that has or is selected for having NSCLC that has failed prior treatment with an immune checkpoint antibody.

266. The combination therapy of any one of the preceding embodiments, wherein the combination therapy is for use in a subject that has or is selected for having NSCLC that has failed prior treatment with chemotherapy.

267. The combination therapy of any one of the preceding embodiments, wherein the combination therapy is for use in a subject that has or is selected for having NSCLC that has failed prior treatment with an anti-PD-1 immune checkpoint antibody.

268. The combination therapy of any one of the preceding embodiments, wherein the combination therapy is for use in a subject that has or is selected for having NSCLC that has failed prior treatment with an PD-L1 immune checkpoint antibody.

269. The combination therapy of any one of the preceding embodiments, wherein the combination therapy is for use in a subject that has or is selected for having NSCLC that has failed prior treatment with an anti-PD-1 or PD-L1 immune checkpoint antibody.

270. The combination therapy of any one of the preceding embodiments, wherein the combination therapy is for use in a subject that has or is selected for having NSCLC that has failed prior treatment with an immune checkpoint antibody and chemotherapy. The combination therapy of any one of the preceding embodiments, wherein the combination therapy is for use in a subject that has or is selected for having NSCLC that has failed prior treatment with an anti-PD-1 immune checkpoint antibody and a platinum-containing doublet treatment.

271. The combination therapy of any one of the preceding embodiments, wherein the combination therapy is for use in a subject that has or is selected for having NSCLC that has failed prior treatment with an anti-PD-L1 immune checkpoint antibody and a platinum-containing doublet treatment.

272. The combination therapy of any one of the preceding embodiments, wherein the combination therapy is for use in a subject that has or is selected for having NSCLC that has failed prior treatment with an anti-PD-1 or PD-L1 immune checkpoint antibody and a platinum-containing doublet treatment.

273. The combination therapy of any one of the preceding embodiments, wherein the combination therapy is for use in a subject that has or is selected for having a PD-L1 tumor proportion score of ≥50%.

274. The combination therapy of any one of the preceding embodiments, wherein the combination therapy is for use in a subject that has or is selected for having a PD-L1 tumor proportion score of <50%.

275. The combination therapy of any one of the preceding embodiments, wherein the combination therapy is for use in a subject that has or is selected for having a PD-L1 tumor proportion score of between 1% to 49%.

276. The combination therapy of any one of the preceding embodiments, wherein the subject has or is selected for having one or more lesions with a PD-L1 tumor proportion score of ≤15%.

277. The combination therapy of any one of the preceding embodiments, wherein the subject has or is selected for having one or more lesions with a PD-L tumor proportion score of <5%.

278. The combination therapy of any one of the preceding embodiments, wherein the subject has or is selected for having one or more lesions with a PD-L1 tumor proportion score of ≤1%.

279. The combination therapy of any one of the preceding embodiments, wherein the combination therapy is for use in a subject that has or is selected for having a PD-L1 tumor proportion score of <1%.

280. The combination therapy of any one of the preceding embodiments, wherein the subject has or is selected for having one or more lesions with no evidence of PD-L1 expression.

281. The combination therapy of any one of the preceding embodiments, wherein the subject has or is selected for having a NSCLC that is not eligible for treatment comprising an immune checkpoint antibody.

282. The combination therapy of any one of the preceding embodiments, wherein the subject has or is selected for having NSCLC and would unlikely benefit from treatment comprising an immune checkpoint antibody.

283. The combination therapy of any one of the preceding embodiments, wherein the tumor proportion score is determined at baseline.

284. The combination therapy of any one of the preceding embodiments, wherein the tumor proportion score is determined prior to administration of the combination therapy.

285. The combination therapy of any one of the preceding embodiments, wherein the combination therapy is for use in a subject that has NSCLC characterized as having a KRAS mutation.

286. The combination therapy of any one of the preceding embodiments, wherein the combination therapy is for use in a subject having NSCLC comprising one or more lesions carrying a KRAS mutation.

287. The combination therapy of any one of the preceding embodiments, wherein the combination therapy is for use in a subject that has or is selected for having a tumor that has poor infiltration of immune cells.

288. The combination therapy of any one of the preceding embodiments, wherein the combination therapy is for use in a subject that has or is selected for having one or more lesions showing signs of an EMT signature.

289. The combination therapy of any one of the preceding embodiments, wherein the combination therapy is for use in a subject that has or is selected for having one or more lesions showing signs of an EMT signature.

290. The combination therapy of any one of the preceding embodiments, wherein the combination therapy is for administration in a cycle of treatment including administration of the anti-clusterin antibody or antigen binding fragment thereof on days 1, 8 and 15 and administration of docetaxel on day 1.

291. The combination therapy of any one of the preceding embodiments, wherein the anti-clusterin antibody or antigen binding fragment thereof is for use as an IV infusion.

292. The combination therapy of any one of the preceding embodiments, wherein docetaxel is for use as an IV infusion.

293. The combination therapy of any one of the preceding embodiments, wherein the clinical benefit is regression or a lesion.

294. The combination therapy of any one of the preceding embodiments, wherein the clinical benefit is a decrease in the size of a lesion. 295. The combination therapy of any one of the preceding embodiments, wherein the clinical benefit is a stabilization in the size of a lesion. 296. The combination therapy of any one of the preceding embodiments, wherein the clinical benefit is a stabilization in the size of a lesion.

297. The combination therapy of any one of the preceding embodiments, wherein the clinical benefit is a reduction in the number of tumor cells in a lesion.

298. The combination therapy of any one of the preceding embodiments, wherein the clinical benefit is defined as per RECIST 1.1 guidelines.

299. The combination therapy of any one of the preceding embodiments, wherein the use procures a clinical benefit for at least 6 weeks after first determination of response to treatment.

300. The combination therapy of any one of the preceding embodiments, wherein the use procures a clinical benefit for at least 12 weeks after first determination of response to treatment.

301. The combination therapy of any one of the preceding embodiments, wherein the use procures a clinical benefit for at least 18 weeks after first determination of response to treatment.

302. The combination therapy of any one of the preceding embodiments, wherein the use procures a clinical benefit for at least 24 weeks after first determination of response to treatment.

303. The combination therapy of any one of the preceding embodiments, wherein the use procures a clinical benefit for at least 30 weeks after first determination of response to treatment.

304. The combination therapy of any one of the preceding embodiments, wherein the use procures a clinical benefit for at least 36 weeks after first determination of response to treatment.

305. The combination therapy of any one of the preceding embodiments, wherein the use procures a clinical benefit for at least 42 weeks after first determination of response to treatment.

306. The combination therapy of any one of the preceding embodiments, wherein the clinical benefit is selected from complete response (CR) as per RECIST 1.1 guidelines, partial response (PR) as per RECIST 1.1 guidelines or stable disease (SD) as per RECIST 1.1 guidelines.

307. The combination therapy of any one of the preceding embodiments, wherein the clinical benefit is partial response (PR) as per RECIST 1.1 guidelines.

308. The combination therapy of any one of the preceding embodiments, wherein the clinical benefit is stable disease (SD) as per RECIST 1.1 guidelines.

309. The combination therapy of any one of the preceding embodiments, wherein the size of the lesion is assessed in accordance with RECIST 1.1 guidelines.

310. The combination therapy of any one of the preceding embodiments, wherein the clinical benefit is a reduction in or an elimination of metabolic activity in a lesion.

311. The combination therapy of any one of the preceding embodiments, wherein the clinical benefit is an improvement in a symptom associated with said cancer.

312. The combination therapy of any one of the preceding embodiments, wherein the combination therapy is for administration as at least two cycles of treatment.

313. The combination therapy of any one of the preceding embodiments, wherein the combination therapy is for use as long as a clinical benefit is observed.

314. The combination therapy of any one of the preceding embodiments, wherein the combination therapy is for use until tumor progression.

315. The combination therapy of any one of the preceding embodiments, wherein the combination therapy is for use until unmanageable toxicity occurs.

316. The combination therapy of any one of the preceding embodiments, wherein the use of the anti-clusterin antibody or antigen binding fragment thereof is maintained after the appearance of clinical benefits.

317. The combination therapy of any one of the preceding embodiments, wherein the anti-clusterin antibody or antigen binding fragment thereof inhibits epithelial to mesenchymal transition.

318. The combination therapy of any one of the preceding embodiments, wherein the anti-clusterin antibody or antigen binding fragment thereof comprises a light chain variable region comprising CDRs having the amino acid sequence set forth in SEQ ID NO:1 (CDRL1), in SEQ ID NO:2 (CDRL2) and in SEQ ID NO: 3 (CDRL3) and a heavy chain variable region comprising CDRs having the amino acid sequence set forth in SEQ ID NO:4 (CDRH1), SEQ ID NO:5 (CDRH2) and SEQ ID NO:6 (CDRH3).

319. The combination therapy of any one of the preceding embodiments, wherein the anti-clusterin antibody or antigen binding fragment thereof comprises a light chain variable region comprising CDRs having the amino acid sequence set forth in SEQ ID NO:1 (CDRL1), in SEQ ID NO:2 (CDRL2) and in SEQ ID NO: 3 (CDRL3) and a heavy chain variable region comprising CDRs having the amino acid sequence set forth in SEQ ID NO:7 (CDRH1), SEQ ID NO:8 (CDRH2) and SEQ ID NO:9 (CDRH3).

320. The combination therapy of any one of the preceding embodiments, wherein the anti-clusterin antibody or antigen binding fragment thereof comprises:
a. a light chain variable region having an amino acid sequence having at least 80% identity with the amino acid sequence set forth in SEQ ID NO:12 and a heavy chain variable region having an amino acid sequence at least 80% identity with the amino acid sequence set forth in SEQ ID NO:13 or;
b. a light chain having an amino acid sequence having at least 80% identity with the amino acid sequence set forth in SEQ ID NO: 14 and a heavy chain having an amino acid sequence having at least 80% identity with the amino acid sequence set forth in SEQ ID NO: 15.

321. The combination therapy of any one of the preceding embodiments, wherein the anti-clusterin antibody or antigen binding fragment thereof comprises a light chain variable region having the amino acid sequence set forth in SEQ ID NO: 12 and a heavy chain variable region having the amino acid sequence set forth in SEQ ID NO:13.

322. The combination therapy of any one of the preceding embodiments, wherein the anti-clusterin antibody or antigen binding fragment thereof comprises a light chain having the amino acid sequence set forth in SEQ ID NO: 14 and a heavy chain having the amino acid sequence set forth in SEQ ID NO:15.

323. The combination therapy of any one of the preceding embodiments, wherein the combination therapy is for use in a subject that is not immunosuppressed or has not received an immunosuppressive medication within 7 days prior to treatment.

324. The combination therapy of any one of the preceding embodiments, wherein the combination therapy is for use in a subject that has not received prior treatment with docetaxel.

325. The combination therapy of any one of the preceding embodiments, wherein the combination therapy is for use in a subject that does not receive concurrent anti-cancer treatment with the anti-clusterin antibody or antigen binding fragment thereof and/or docetaxel combination treatment.

326. The combination therapy of any one of the preceding embodiments, wherein the combination therapy is for use in humans.

327. The combination therapy of any one of the preceding embodiments, wherein the combination therapy is for in a subject having a functional immune system.

328. The combination therapy of any one of the preceding embodiments, wherein the combination therapy is for in a subject having an adequate organ and immune function.

329. The combination therapy of any one of the preceding embodiments, wherein the combination therapy is for use as a first-line therapy.

330. The combination therapy of any one of the preceding embodiments, wherein the combination therapy is for use as a second-line therapy.

331. The combination therapy of any one of the preceding embodiments, wherein the combination therapy is for treatment of non-small cell lung cancer.

332. The combination therapy of any one of the preceding embodiments, wherein the NSCLC is metastatic NSCLC.

333. The combination therapy of any one of the preceding embodiments, wherein the metastatic NSCLC is stage III NSCLC.

334. The combination therapy of any one of the preceding embodiments, wherein the metastatic NSCLC is stage IV NSCLC.

335. The combination therapy of any one of the preceding embodiments, wherein the metastatic NSCLC is a stage III to IV NSCLC.

336. The combination therapy of any one of the preceding embodiments, wherein the subject having NSCLC is a subject having metastatic NSCLC.

337. The combination therapy of any one of the preceding embodiments, wherein the subject having metastatic NSCLC is a subject having stage III NSCLC.

338. The combination therapy of any one of the preceding embodiments, wherein the subject having metastatic NSCLC is a subject having stage IV NSCLC.

339. The combination therapy of any one of the preceding embodiments, wherein the subject has or is selected for having a stage III to IV NSCLC.

340. The combination therapy of any one of the preceding embodiments, wherein the combination therapy is for obtaining a clinical benefit.

341. The combination therapy of any one of the preceding embodiments, wherein the combination therapy promotes infiltration of immune cells in the tumor microenvironment of one or more lesions.

342. The combination therapy of any one of the preceding embodiments, wherein the combination therapy is for increasing response to immunotherapy subsequently to combination therapy.

343. The combination therapy of any one of the preceding embodiments, wherein the lesion is a tumor metastasis.

344. The combination therapy of any one of the preceding embodiments, wherein the lesion is the primary tumor.

345. The combination therapy of any one of the preceding embodiments for use in the method of any of the preceding embodiments.

346. The combination therapy of any one of the preceding embodiments, wherein one or more lesions have a PD-L1 tumor proportion score (TPS) of ≤15%.

347. The combination therapy of any one of the preceding embodiments, wherein one or more lesions have a PD-L1 tumor proportion score (TPS) of <5%.

348. The combination therapy of any one of the preceding embodiments, wherein one or more lesions have a PD-L1 tumor proportion score (TPS) of ≤1%.

349. The combination therapy of any one of the preceding embodiments, wherein one or more lesions have a PD-L1 tumor proportion score (TPS) of <1%. 350. The combination therapy of any one of the preceding embodiments, wherein one or more lesions have a PD-L1 combined positive score (CPS) of <10%, 351. The combination therapy of any one of the preceding embodiments, wherein one or more lesions have a PD-L1 combined positive score (CPS) of <5%.

352. The combination therapy of any one of the preceding embodiments, wherein one or more lesions have a PD-L1 combined positive score (CPS) of <1%.

353. The combination therapy of any one of the preceding embodiments, wherein PD-L1 expression is undetectable or non-evaluable.

354. The combination therapy of any one of the preceding embodiments, wherein one or more lesions is characterized as being immunologically cold.

355. The combination therapy of any one of the preceding embodiments, wherein the combination therapy is provided as a first-line therapy.

356. The combination therapy of any one of the preceding embodiments, wherein the combination therapy is provided as a second-line therapy.

357. The combination therapy of any one of the preceding embodiments, wherein the combination therapy is provided as a subsequent therapy.

### Examples

### Example 1-PHASE II CLINICAL STUDY DESIGN

The Applicant is evaluating the use of anti-clusterin antibodies combined with docetaxel in previously treated subjects with metastatic non-small cell lung cancer.

This Phase II study recruits 40 metastatic non-small cell lung cancer patients who failed treatment with a platinum-containing doublet treatment and an anti-PD-1 or PD-L1 immune checkpoint antibody, administered simultaneously or sequentially. All recruited patients receive AB-16B5 (herein referred to as humanized 16B5) at a dose of 12 mg/kg once weekly combined with docetaxel at a dose of 75 mg/m² once every 3 weeks.

### Objectives

A primary objective of this study is to determine the objective response rate (ORR) per RECIST 1.1 (Eisenhauer, E. A., et al., European Journal of Cancer 45:228-247 (2009)) in subjects receiving the combination of AB-16B5 and docetaxel.

Another primary objective of this study is to determine the safety and tolerability of the combination of AB-16B5 and docetaxel.

A secondary objective of this study is to determine the clinical benefit rate (complete response (CR), partial response (PR) and stable disease (SD)) per RECIST 1.1 in subjects receiving the combination of AB-16B5 and docetaxel.

Another secondary objective of this study is to determine the duration of response (CR and PR) per RECIST 1.1 in subjects receiving the combination of AB-16B5 and docetaxel.

Yet another secondary objective of this study is to determine the duration of stable disease per RECIST 1.1 in subjects receiving the combination of AB-16B5 and docetaxel.

A further secondary objective of this study is to determine the progression free survival (PFS) per RECIST 1.1 in subjects receiving the combination of AB-16B5 and docetaxel.

Another secondary objective of this study is to determine the overall survival (OS) in subjects receiving the combination of AB-16B5 and docetaxel.

Yet another secondary objective of this study is to determine the pharmacokinetics of AB-16B5 in this subject population.

An exploratory objective of this study is to perform exploratory pharmacodynamic evaluation of the effect of the combination of AB-16B5 and docetaxel on epithelial to mesenchymal transition (EMT) biomarkers, immune cell biomarkers and immune checkpoints in tumor biopsies.

An exploratory objective of this study is to evaluate disease response using iRECIST in subjects pursuing treatment beyond progression.

### Study Design

The study is an open-label, single-arm, multi-center Phase II trial of AB-16B5 in combination with docetaxel in previously treated subjects with metastatic non-small cell lung cancer who have experienced disease progression following treatment with a platinum-containing doublet treatment and an anti-PD-1 or PD-L1 immune checkpoint antibody, administered simultaneously or sequentially. Approximately 40 subjects are enrolled in this trial and receive AB-16B5 at a dose of 12 mg/kg once weekly on Days 1, 8 and 15 combined with docetaxel at a dose of 75 mg/m² once every 3 weeks on Day 1. One cycle of treatment consists of 21 days (3 weeks). The safety profile of the AB-16B5 and docetaxel combination is examined during a safety lead-in period with the first 8 subjects completing one cycle of treatment.

Subjects are evaluated every 6 weeks with radiographic imaging to assess response to treatment using Response Evaluation Criteria in Solid Tumors (RECIST) 1.1 criteria for determination of the objective response rate (ORR) and progression free survival (PFS). A futility analysis is conducted to minimize subject exposure to an ineffective treatment. Paired tumor biopsies (pre-treatment and on-treatment) are collected in all subjects. Adverse events are monitored throughout the study and graded for severity according to the NCI Common Terminology Criteria for Adverse Events (CTCAE). Study treatment continues until there is evidence of disease progression (defined according to RECIST 1.1), treatment-related adverse events of unacceptable severity, subject request for discontinuation or Investigator determination that further treatment is not in the subject's best interest. Treatment beyond progression is allowed if the Investigator considers the subject to be clinically stable. Subjects who must discontinue docetaxel due to toxicity continue treatment with AB-16B5.

### Safety Lead-In Period

The safety profile of AB-16B5 at a dose of 12 mg/kg administered once weekly on Days 1, 8 and 15 combined with docetaxel at a dose of 75 mg/m² once every 3 weeks on Day 1 is examined during a safety lead-in period with the first 8 subjects completing one cycle of treatment. Decision to de-escalate the dose of AB-16B5 can be made using the modified toxicity probability interval method (mTPI).

The study treatment is considered acceptable if no more than 3 dose limiting toxicity (DLTs) are observed during the first cycle in the first 8 subjects treated.

For these purposes, a DLT is defined as a Grade ≥3 non-hematologic toxicity occurring during Cycle 1 of therapy. In addition, the following hematologic toxicities are considered as a DLT:
Grade ≥4 neutropenia or thrombocytopenia >7 days
Grade ≥3 thrombocytopenia with bleeding
Grade ≥3 febrile neutropenia

Toxicities that are clearly and incontrovertibly due to disease progression or to extraneous causes are not to be considered DLTs. In addition, the following non-hematologic toxicities are not to be considered DLTs:
Grade 3 arthralgia or myalgia which returns to Grade≤1 within <7 days with appropriate supportive care
Grade 3 nausea, vomiting or diarrhea which returns to Grade≤1 within <72 hours with appropriate supportive care
Grade 3 fatigue lasting <7 days
Grade 3 electrolyte abnormalities that last less than 72 hours and are not associated with clinical symptoms
Grade 3 amylase or lipase elevation which is not associated with symptoms or clinical manifestations of pancreatitis

De-escalation of AB-16B5 is performed if more than 3 DLTs are observed in the first 8 subjects treated.

In such a case, the next three subjects are treated with AB-16B5 at 9 mg/kg AB-16B5 administered once weekly on Days 1, 8 and 15 combined with docetaxel at a dose of 75 mg/m² once every 3 weeks on Day 1. If 0 or 1 DLT is observed during the first cycle in these 3 subjects, AB-16B5 at the 9 mg/kg dose is considered acceptable.

If more than 1 DLTs are observed, a final de-escalation of AB-16B5 to 6 mg/kg is performed and the safety profile is evaluated using the same process as described above.

### Inclusion Criteria

Subjects enrolled in the study meet the following inclusion criteria:
Subjects (male or non-pregnant female) having >18 years of age on the day of signing the informed consent.
Subjects having a histologically or cytologically confirmed diagnosis of (Stage III-IV) non-small cell lung cancer (NSCLC) and with at least one measurable lesion defined by RECIST 1.1.
Subjects having experienced a disease progression following treatment with a an anti-PD-1 or PD-L1 immune checkpoint antibody and a platinum-containing doublet treatment, administered simultaneously or sequentially.
Subjects having targetable driver mutation in EGFR or ALK gene are allowed on trial after failing available targeted therapies and having experienced a disease progression following treatment with an anti-PD-1 or PD-L1 immune checkpoint antibody and a platinum-containing doublet treatment, administered simultaneously or sequentially.
Subjects having adequate organ and immune function as indicated in Table 1 below:

| | |
|---|---|
| System | Laboratory Value |

Hematological

| | |
|---|---|
| Absolute neutrophil count (ANC) | ≥1.5 × 10⁹/L (1,500 cells/mm³) |
| Platelets | ≥100 × 10⁹/L (100,000 cells/mm³) |
| Hemoglobin | ≥90 g/L |

Hepatic

| | |
|---|---|
| Total bilirubin | ≤ULN |
| AST (SGOT) and ALT (SGPT) | ≤1.5 × ULN, unless secondary to |
| | liver metastases |
| Alkaline phosphatase | ≤2.5 × ULN |

Renal

| | |
|---|---|
| Creatinine clearance^{a} | ≥60 mL/min |

Coagulation

| | |
|---|---|
| International Normalized Ratio | ≤1.5 × ULN^{b} |
| (INR) | |

^{a}Creatinine clearance should be calculated using the Cockcroft-Gault Method: CrCl = [(140-age) × weight (kg) × (0.85 for females only)]/(72 × serum creatinine)
^{b}if subject is receiving anticoagulant therapy, then INR must be within therapeutic range.

Subjects having a tumor lesion amenable for biopsies with no contraindication for biopsy.
Subjects having an Eastern Cooperative Oncology Group (ECOG) performance status of ≤2.
Subjects having a life expectancy of at least 3 months.
Subjects having recovered from the toxic effects resulting from the most recent cancer treatment to Grade 1 or less. If the subjects underwent major surgery or received radiation therapy, they have recovered from the complications and/or toxicity.
Female subjects of childbearing potential having a negative urine or serum pregnancy test within 72 hours prior to the first dose of study treatment. If the urine test is positive or cannot be confirmed as negative, a serum pregnancy test will be required. The serum pregnancy test should preferably be negative for the subject to be eligible.
Subjects (both male and female) of reproductive potential are willing to practice highly effective methods of contraception throughout the study and for up to 90 days after the last dose of study medication. Abstinence is acceptable if this is the subject's usual lifestyle.
Female subjects are not considered of childbearing potential if they have a history of surgical sterility or evidence of post-menopausal status defined as any of the following:
   ≥45 years of age and has not had menses for more than 2 years.
   Amenorrhoeic for <2 years without hysterectomy and oophorectomy and a follicle stimulating hormone (FSH) value in the postmenopausal range at screening.
   Post hysterectomy, oophorectomy or tubal ligation. Documented hysterectomy or oophorectomy must be confirmed with medical records of the actual procedure or by ultrasound. Tubal ligation must be confirmed with medical records of the actual procedure.

It is to be understood herein that the inclusion criteria are for the purpose of the clinical trial only and are not to be considered limitations of the approved drug for treatment.

### Exclusion Criteria

Subjects enrolled in the study meet the following exclusion criteria:
Subjects having received prior therapy with AB-16B5.
Subjects having received prior therapy with docetaxel for the treatment of NSCLC.
Subjects who are currently participating or has participated in a study of an investigational agent or using an investigational device within 21 days prior to the first dose of study treatment. The 21-day window should be calculated using the last dose of an antineoplastic investigational agent or last use of an investigational device with antineoplastic intent.
Subjects who have received any anti-cancer treatment within 3 weeks or radiation therapy within 2 weeks prior to receiving the first dose of study treatment or who have not recovered from adverse events to Grade 1 or less. Subjects with alopecia are eligible to participate.
Subjects who are expected to require any other form of systemic or localized antineoplastic therapy while on the trial. This includes maintenance therapy with another agent or radiation therapy.
Subjects who are receiving a dose >10 mg/day of prednisone (or equivalent) within 7 days prior to the first dose of study treatment or any other form of immunosuppressive medication (corticosteroid pre-treatment and/or post-treatment of docetaxel is allowed).
Subjects who require treatment with a strong CYP3A4 inhibitor (e.g., ketoconazole, itraconazole, clarithromycin, atazanavir, indinavir, nefazodone, nelfinavir, ritonavir, saquinavir, telithromycin and voriconazole). Subjects may be included if there is an alternate treatment with a weak CYP3A4 inhibitor and they are willing to change prior to randomization. If the subject agrees to change from a strong inhibitor to a weak CYP3A4 inhibitor, the strong inhibitor must be stopped at least 7 days prior to the first dose of study treatment.
Subjects who have another malignancy that is progressing or requires active treatment. Exceptions include basal cell carcinoma of the skin, squamous cell carcinoma of the skin or in situ cervical cancer.
Subjects who have known active central nervous system metastases and/or carcinomatous meningitis. Subjects with previously treated brain metastases may participate if they have been clinically stable for at least 2 weeks prior to the first dose of study treatment, if they have no evidence of new or enlarging brain metastases and if they are not receiving a dose >10 mg/day of prednisone (or equivalent) within 7 days prior to the first dose of study treatment.
Subjects with clinically significant ECG abnormalities.
Subjects who have received or will receive a live vaccine within 30 days prior to the first dose of study treatment.
Subjects with a known history of human immunodeficiency (HIV).
Subjects with an active Hepatitis B or C infection.
Subjects with an active infection requiring antibiotic therapy.
Subjects with a known history of alcohol or other substance abuse within the last year.
Subjects with known hypersensitivity to docetaxel or drugs formulated with polysorbate 80.
Subjects who have a history or current evidence of any condition, therapy or laboratory abnormalities that may confound the results of the trial, interfere with the subject's participation for the full duration of the trial or if it is not in the best interest of the subject to participate in the trial.
Subjects with medical, social or psychosocial factors that, in the opinion of the treating Investigator, could impact the safety or compliance with study procedures.
Subjects who are pregnant or lactating or who are expecting to conceive or father children within the projected duration of the trial through 90 days after the last dose of AB-16B5 or the last dose of docetaxel.

It is to be understood herein that the exclusion criteria are for the purpose of the clinical trial only and are not to be considered limitations of the approved drug for treatment.

### Study Treatment Study Drug, Dose and Mode of Administration

### AB-16B5

AB-16B5 is a humanized IgG₂ monoclonal antibody targeting sCLU for the inhibition of cancer-associated EMT (humanized 16B5). AB-16B5 is provided in 10 ml vials at a protein concentration of 10.0 mg/mL. AB-16B5 is formulated in a citrate buffer solution at pH 6.0. AB-16B5 vials are stored upright at 2-8° C.

Subjects will receive AB-16B5 by a 60-minute IV infusion once weekly (refer to Pharmacy Manual for the infusion conditions) on Days 1 (prior to docetaxel infusion), 8 and 15. The dose of AB-16B5 will be determined during the safety lead-in period.

Subjects who experience infusion-related reactions will be treated with corticosteroids such as dexamethasone. Antihistamines and acetaminophen can also be used, as deemed appropriate.

Premedication to prevent infusion reaction related to AB-16B5 will not be employed initially. Subjects who have experienced infusion-related reactions will be premedicated as follows:
Grade 1 (mild): No premedication at subsequent dosing
Grade 2 (moderate): Dexamethasone 8 mg PO BID the day prior to AB-16B5 infusion and acetaminophen 650 mg PO and diphenhydramine 25-50 mg PO 30-60 minutes prior to AB-16B5 infusion
Grade 3 (severe) and Grade 4 (life-threatening): Subject will be discontinued from further treatment with AB-16B5

At any point during the study, if clinically significant infusion related reactions are observed in multiple subjects, the Investigator could decide in agreement with the Sponsor to implement a premedication for all new subjects.

### Docetaxel

Docetaxel will be administered at the dosage of 75 mg/m² by a 60-minute IV infusion once every 3 weeks on Day 1. Docetaxel will be prepared and administered as per the approved product label/monograph.

All subjects should be premedicated with corticosteroids as per hospital standard practices. Vein extravasation and accidental spillages should be dealt with according to the hospital standard practices.

### Treatment Duration

One cycle of treatment will consist of 21 days (3 weeks).

Study treatment will continue until there is evidence of disease progression, unacceptable toxicity, subject requests discontinuation of study treatment, or the Investigator feels that further treatment is not in the subject's best interest. Subjects who must discontinue docetaxel due to toxicity will continue on AB-16B5.

Treatment beyond progression will be allowed if the Investigator considers the subject to be clinically stable. The clinical judgment decision by the site should be based on the clinical stability of the subjects as defined below:

Clinical stability is defined as the following:
Absence of symptoms and signs indicating clinically significant progression of disease, including worsening of laboratory values
No decline in ECOG performance status
Absence of rapid progression of disease
Absence of progressive tumor at critical anatomical sites (e.g., cord compression) requiring urgent alternative medical intervention

Any subject deemed clinically unstable should be discontinued from trial treatment at first radiologic evidence of PD.

Study Treatment, Dose Reduction after Safety Lead-In

AB-16B5 Dose Reduction

Subjects who experience any Grade ≥3 adverse event that is judged to be possibly, probably or likely related to AB-16B5 and not requiring treatment discontinuation should have a reduction in AB-16B5 dose by one dose level (Table 2 below).

Treatment will be re-initiated at the lower dose only after recovery of the adverse event to Grade≤1.

| Starting Dose Level | Reduced Dose |
|---|---|
| 12 mg/kg | 9 mg/kg |
| 9 mg/kg | 6 mg/kg |
| 6 mg/kg | Discontinue AB-16B5 |

Subjects whose original AB-16B5 dose has been reduced for toxicity will not be re-escalated.

### Docetaxel Dose Reduction

Subjects who experience either febrile neutropenia, neutrophils <500 cells/mm³ for more than one week, severe or cumulative cutaneous reactions, or other Grade ≥3 non-hematological toxicities that is judged to be related to docetaxel should have treatment withheld until resolution of the toxicity and then resumed at 60 mg/m². Based upon subject's condition, treatment with AB-16B5 may continue during this period.

A dose of 50 mg/m² once every two weeks has been found as effective as a dose of 75 mg/m² once every three weeks as second-line chemotherapy for advanced non-small cell lung cancer (De Petris, L et al., Anticancer Research November 2005, 25:4713-4717).

In addition, although not part of the clinical protocol, the literature teaches that in subjects who experience febrile neutropenia, the dose of docetaxel may be reduced to 65 mg/m² or even to 50 mg/m² by the physician.

Subjects who develop Grade ≥3 peripheral neuropathy should have docetaxel discontinued.

### Example 2-PHASE II CLINICAL STUDY-PRELIMINARY RESULTS

The safety lead-in treatment period confirmed that a dose of AB-16B5 of 12 mg/kg once per week and a dose of docetaxel of 75 mg/m² is safe. It is thus expected that lower dosage could also be safe.

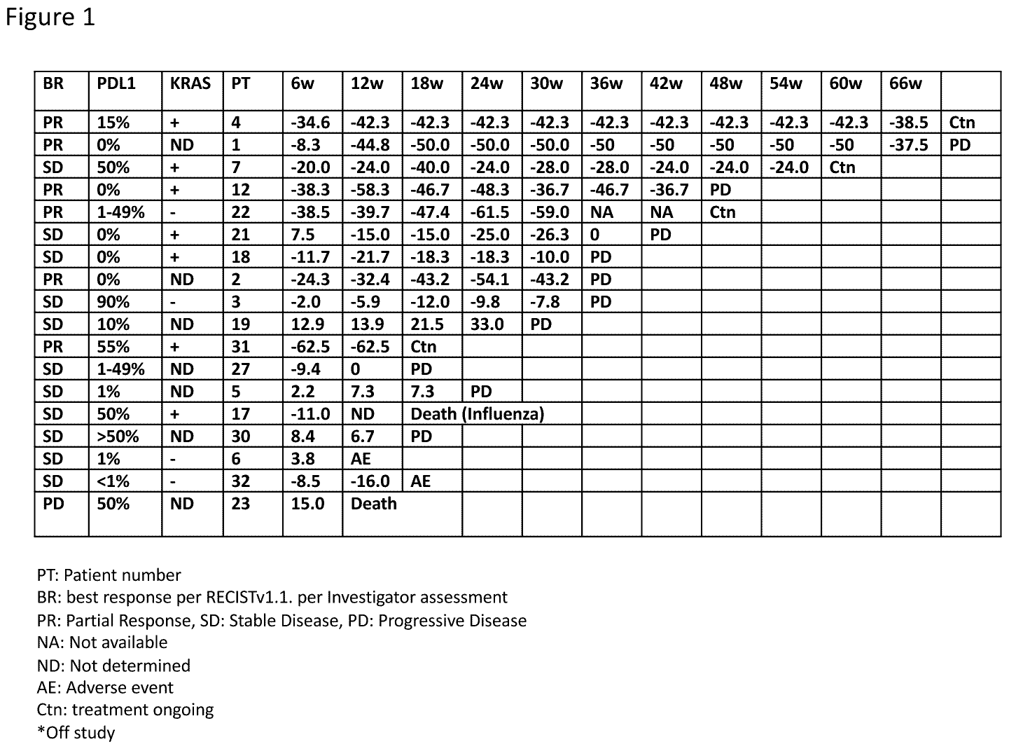

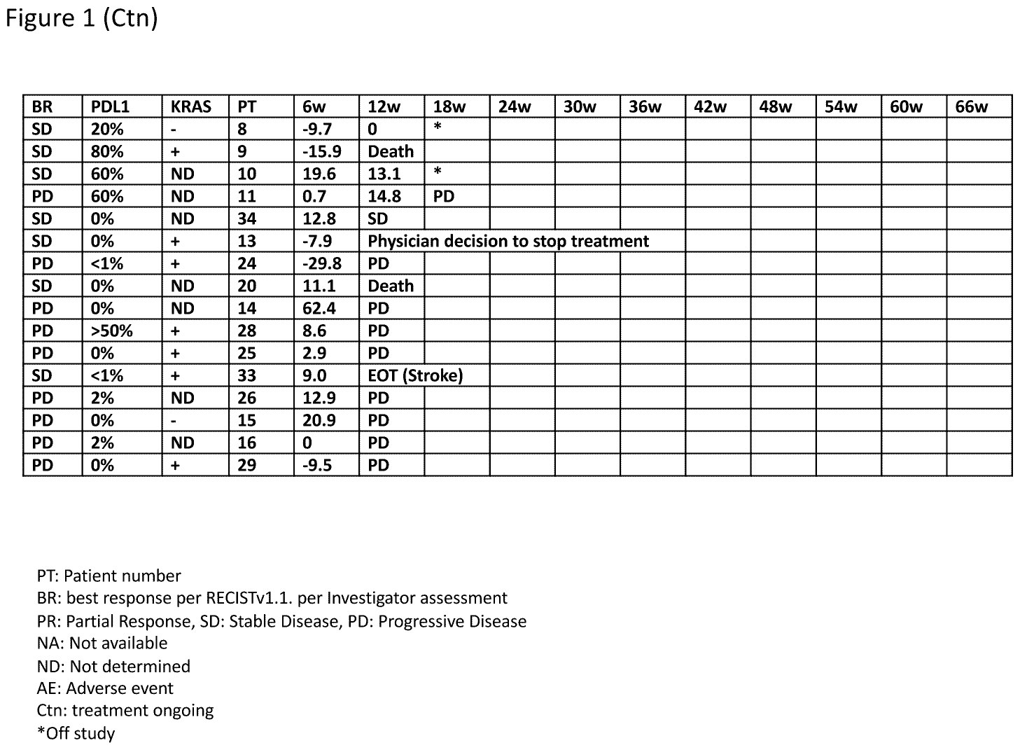

FIG. 1 is a chart showing the duration of patients on treatment as the best response as per RECIST 1.1 with 6 patients showing partial response (PR), 18 patients showing stable disease (SD) and 10 patients showing progressive diseases (PD) in 34 evaluable patients and also presents the measurement of lesions at the indicated time of treatment as per RECISTv1.1.

Among the patients who received AB-16B5 combined with docetaxel for more than 12 weeks (19 patients), the best response was 6 partial response (PR), 12 stable disease (SD) and one progressive disease (PD). KRAS mutation was assessed in several patients' tumors prior to first line therapy. Among these 19 patients, tumors from 7 patients (4, 7, 12, 17, 18, 21) had a KRAS mutation; 5 tumors were KRAS negative (3, 6, 8, 22 and 32) and KRAS status was not determined for the 7 other patients (1, 2, 5, 19, 23, 27 and 30) (FIG. 1).

As can be seen from FIG. 1, 4 NSCLC patients out of 5 (Patient 1, 2, 4, and 12) showing evidence of partial response have low expression of PD-L1 ((TPS) of ≤15%).

Biopsies from some patients under treatment were obtained for analysis of various parameters. H&E stains were analyzed by a certified pathologist. Detections of tumor cell markers and immune cells were performed on successive histological sections. Slides were digitized and analyzed using algorithms created using open-source software QuPath™.

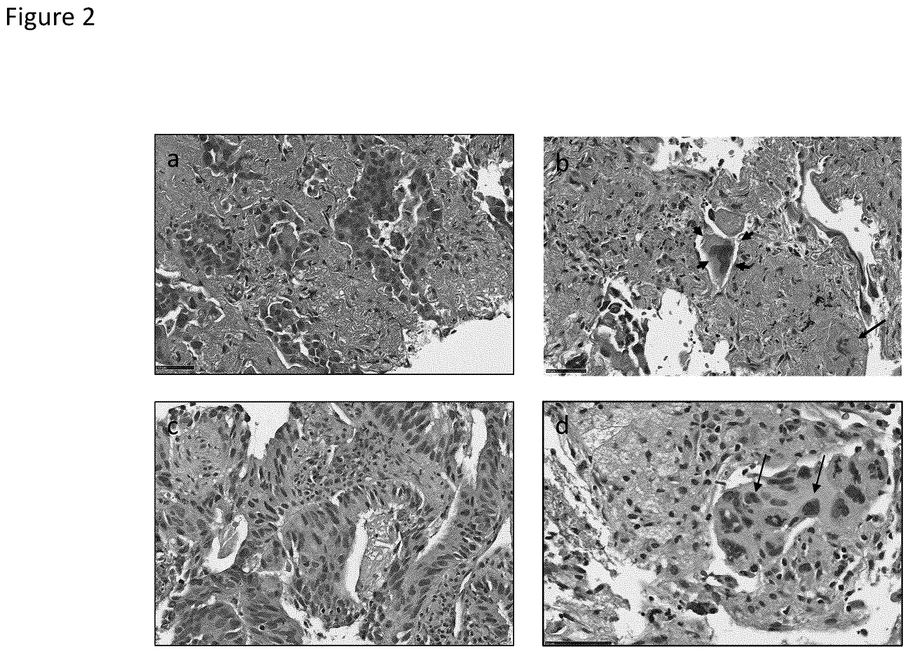

Histology analyses of pre- and on-treatment biopsies from two patients under study are illustrated in FIGS. 2a-2d. Tumor sections were stained with hematoxylin and eosin. The pre-treatment samples are largely composed of neoplastic tumor cells with no evidence of inflammatory cells (a and c). In contrast, very few tumor cells could be detected in the on-treatment samples (b and d). In addition, cytopathic changes caused by docetaxel were observed (arrows in b and d) as well as a brisk inflammatory infiltrate.

### Patient 1 (PTI)

The PD-L1 TPS score determined during the first-line therapy was 0%. Results from a scan after the eight cycles of treatment indicated a 50% decrease in the size of the target lesions. This patient therefore showed evidence of partial response.

Analysis of the pre-treatment biopsy revealed a modest lymphocytic infiltration in one specimen while fibrosis and a chronic inflammatory infiltrate was present in another one. However, the largest portion of another core was replaced by neoplastic tumor cells with massive necrosis. No residual tumor cells could be found on any of the four cores from the on-treatment biopsy. Fibrotic tissue with chronic lymphoplasmacytic inflammation was observed in on-treatment tissue cores. Lymphocytes and plasma cells infiltration was found to be pronounced in certain areas. On-treatment samples fail to demonstrate residual tumor cells. Of note, fibrosis and chronic inflammation are present in on-treatment cores. Tumor cells were noted only in pre-treatment conditions.

Analysis of the Ki-67 proliferation biomarker indicated a dense staining in the pre-treatment condition and a strong reduction of the signal in the on-treatment samples. Image analyses revealed a decrease of 42% of proliferative tumor cells after the second cycle of treatment.

Image analysis of E-cadherin staining in the pre-treatment biopsy revealed a large number of tumor cells with an H-score of 166. No E-cadherin staining could be observed in the on-treatment biopsy suggesting that the treatment was effective in eradicating the tumor cells.

Image analysis of CD3 staining in the pre-treatment biopsy revealed that the tumor was poorly infiltrated with T lymphocytes before initiation of treatment. However, a 45% increase of CD3 signal was observed in the on-treatment biopsy.

Image analysis of CD4 staining showed an 100% increase of CD4 T cells in the on-treatment biopsy which increased from 4% to 8% of cells in the region of interest.

Image analysis of CD8 staining showed an increase of 86% of CD8 T cells in the on-treatment biopsy which increased from 7% to 13% of cells in the region of interest.

### Patient 2 (PT2)

The PD-L1 TPS score determined during the first-line therapy was 0%. The patient has stopped receiving docetaxel at week 33. CT scans were performed after the second, fourth, sixth, eighth and tenth cycle indicating a decrease of the size of the target lesions from 24% at the end the cycle 2 to 43% at the end of cycle 10. This patient therefore shows evidence of partial response.

Analysis of tumor biopsies revealed that pre-treatment samples are more basophilic reflecting the presence of numerous tumor cells aggregates. In contrast, samples from the on-treatment (end of cycle 2) showed large eosinophilic areas reflecting extensive stromal fibrosis. Higher power view of pre-treatment condition showed widespread tumor involvement. Of note, tumor nuclei display cytological abnormalities but no alteration to suggest recent exposure to chemotherapy. There was no inflammatory reaction surrounding neoplastic foci. In on-treatment condition, important findings were reported: cytopathic effects, fibrosis and chronic lymphoplasmacytic and histiocytic inflammation. There were areas of dense sclerotic tissue with chronic inflammatory cells and few residual tumor cells with cytopathic changes consistent with chemotherapeutic agent acting on cellular spindle. Large aneuploid and giant cells are reminiscent of changes induced by docetaxel.

Analysis of the Ki-67 proliferation biomarker indicated a dense staining in the pre-treatment condition and a strong reduction of the signal in the on-treatment samples. Image analyses revealed a decrease of 67.2% of proliferative cells after the second cycle of treatment.

Image analysis of E-cadherin staining in the pre-treatment biopsy revealed a large number of tumor cells with an H-score of 166. No E-cadherin staining could be observed in the on-treatment biopsy suggesting that the treatment was effective in eradicating the tumor cells.

Image analysis of CD3 staining in the pre-treatment biopsy revealed that the tumor was poorly infiltrated with T lymphocytes before initiation of treatment. However, a 304% increase of CD3 signal was observed in the on-treatment biopsy.

Image analysis of CD4 staining showed an 100% increase of CD4 T cells in the on-treatment biopsy which increased from 4% to 8% of cells in the region of interest.

Image analysis of CD8 staining showed an increase of 86% of CD8 T cells in the on-treatment biopsy which increased from 7% to 13% of cells in the region of interest.

### Patient 3 (PT3)

The PD-L1 TPS score determined during the first-line therapy was 90%. The patient remained on first-line treatment for 14 months until tumor progression. Results from CT-scan after the sixth cycle of treatment indicated a 12% decrease in the size of the tumor lesions. This patient therefore shows evidence of stable disease.

### Patient 4 (PT4)

The PD-L1 TPS score determined during the first-line therapy was 15%. Results from CT-scan indicated a 34.6% and 42% decrease in the size of the tumor lesions after two and four cycles of treatment respectively. This patient therefore shows evidence of a long-lasting partial response (72 weeks at time of disclosure).

These data show that the combination therapy, dosage, treatment regimen and/or schedule of administration of the present disclosure is safe and show signs of efficacy. These data also show that the combination therapy, dosage and treatment regimen dosage, treatment regimen and/or schedule of administration of the present disclosure is tolerated for as long as several months with some patients being treated for as long as 66 weeks.

In fact, despite having failed prior treatment with an anti-PD-1 or PD-L1 immune checkpoint antibody and a platinum-containing doublet treatment, the combination therapy, dosage and treatment regimen of the present disclosure procures a clinical benefit to several patients including some having lesions with a PD-L1 tumor proportion score of <50% and <1% and some with tumors carrying a KRAS mutation.

Moreover, as can be seen from FIG. 1, in some patients, the clinical benefit is observed after only two cycles of treatment and lasts for at least 6 weeks and more after first determination of response to treatment.

The combination therapy of the present disclosure may thus be used, for example, in NSCLC patients that progressed after treatment comprising an immune checkpoint antibody (e.g., alone or in combination with chemotherapy).

The relatively poor immune cell infiltration combined with low expression of PD-L1 may explain in part, the failure of treatment comprising an immune checkpoint antibody in NSCLC patients. It is conceivable that the combination therapy of the present disclosure may benefit to NSCLC patients (e.g., metastatic NSCLC patients) having no evidence of PD-L1 expression of with low expression of PD-L1. The combination therapy of the present disclosure may therefore be administered to NSCLC patients having a PD-L1 TPS of ≤15% in lieu of a treatment comprising immune checkpoint inhibitors.

In addition, the combination treatment of the present disclosure may be administered to NSCLC patients that are not eligible for or would unlikely benefit from treatment with an immune checkpoint inhibitor.

Preliminary pharmacokinetics data appears to show accumulation of the anti-clusterin antibody in the treated subjects over time (not shown). This observation may suggest the feasibility of administering the anti-clusterin antibody every two weeks, every three weeks, every month or using lower dose of the anti-clusterin antibody such as between approximately 3 mg/kg to approximately 11 mg/kg. This modification in the treatment regimen may be carried for subsequent cycles or for both the initial cycles and subsequent cycles of treatment.

The present application claims priority to U.S. provisional application No. 63/307,533 filed on Feb. 7, 2022, and to U.S. provisional application No. 63/333,597 filed on Apr. 22, 2022, the entire content of which is incorporated herein by reference.

The embodiments and examples described herein are illustrative and are not meant to limit the scope of the claims. Variations of the foregoing embodiments, including alternatives, modifications and equivalents, are intended by the inventors to be encompassed by the claims. Citations listed in the present application are incorporated herein by reference.

### REFERENCES

Al-Lazikani et al., Standard conformations for the canonical structures of immunoglobulins. J Mol Biol 273:927-948, 1997.
Brochet et al. IMGT/V-QUEST: the highly customized and integrated system for IG and TR standardized V-J and V-D-J sequence analysis. Nucl Acids Res 36: W503-W508, 2008.
Andrew C. R. Martin, Antibody Engineering Vol. 2, Chapter 3: Protein Sequence and Structure Analysis of Antibody Variable Domains. R. Kontermann and S. Dubel (eds.), DOI 10.1007/978-3-642-01147-4_3, #Springer-Verlag Berlin Heidelberg 2010
Shibue, T., Weinberg, R. EMT, CSCs, and drug resistance: the mechanistic link and clinical implications. Nat Rev Clin Oncol 14:611-629 (2017).
Terry, S., Savagner, P., Ortiz-Cuaran, S., Mahjoubi, L., Saintigny, P., Thiery, J.-P. and Chouaib, S., New insights into the role of EMT in tumor immune escape. Mol Oncol, 11:824-846 (2017).
Lenferink, A., Cantin, C., Nantel, A. et al. Transcriptome profiling of a TGF-β-induced epithelial-to-mesenchymal transition reveals extracellular clusterin as a target for therapeutic antibodies. Oncogene 29:831-844 (2010).
New response evaluation criteria in solid tumours: Revised RECIST guideline (version 1.1)” E. A. Eisenhauer, P. Therasse, J. Bogaerts, L. H. Schwartz, D. Sargent, R. Ford, J. Dancey, S. Arbuck, S. Gwyther, M. Mooney, L. Rubinstein, L. Shankar, L. Dodd, R. Kaplan, D. Lacombe, J. Verweij; Eur J Cancer, 45 (2009) 228-247.
Cristiano Ferrario, Julie Laurin, Leon Van Kempen, Caroline Lambert, Alan Spatz, Oksana Markova, Gerald Batist, Adrian Langleben, Mario Filion, Jacques Jolivet. Phase 1 first-in-human study of anti-clusterin antibody AB-16B5 in patients with advanced solid malignancies [abstract]. In: Proceedings of the American Association for Cancer Research Annual Meeting 2017; 2017 Apr. 1-5; Washington, DC. Philadelphia (PA): AACR; Cancer Res 2017; 77 (13 Suppl): Abstract nr CT098. doi: 10.1158/1538-7445.AM2017-CT098.
Hodge, J. W. Garnett, C. T., Farsaci, B., et al. Chemotherapy-induced immunogenic modulation of tumor cells enhances killing by cytotoxic T lymphocytes and is distinct from immunogenic cell death. Int. J. Cancer. 133:624-636 (2013).
Jiang X, Dudzinski S, Beckermann K E, et al. MRI of tumor T cell infiltration in response to checkpoint inhibitor therapy. Journal for ImmunoTherapy of Cancer 2020; 8: e000328. doi: 10.1136/jitc-2019-000328.

MacCallum, R. M., Martin, A. C. R. and Thornton, J. T. ‘Antibody-antigen interactions: Contact analysis and binding site topography’ J. Mol. Biol. 262:732-745, 1996.

Ojima I., Brendan, L., Siyeon, L., Wang, C. and Wang, X., Taxane anticancer agents: a patent perspective, Expert Opin Ther Pat. 2016: 26 (1): 1-20
Seymour, L. et al., iRECIST: Guidelines for response criteria for use in trials testing immunotherapeutics, Lancet Oncol. 18 (3) e143-e152, 2017.
Tremblay G. B., Filion, M., and Sulea, T., Anti-Clusterin Antibodies and Antigen Binding Fragments and their Use to Reduce Tumor Volume, international application No. PCT/CA2010/001882 filed on Nov. 24, 2010 and published on Jun. 3, 2011 under number WO2011/063523 A1.
Wu and Kabat, An analysis of the sequences of the variable regions of Bence Jones proteins and myeloma light chains and their implications for antibody complementarity. J Exp Med 132:211-250, 1993.
Chi, K. N., et al., Randomized Phase II Study of Docetaxel and Prednisone With or Without OGX-011 in Patients With Metastatic Castration-Resistant Prostate Cancer, J Clin Oncol. 28:4247-4254, 2010.
Zhou, J. et al., Therapeutic targeting of myeloid-derived suppressor cells involves a novel mechanism mediated by clusterin, Scientific Reports, 6:29521, 2016.
Radvanyl, L. G., Tumor-Infiltrating lymphocyte therapy: addressing prevailing questions, The Cancer Journal 21 (6): 450-464, 2015.
Romeo, E. et al., The Vicious Cross-Talk between Tumor Cells with an EMT Phenotype and Cells of the Immune System, Cells: 8 (5): 460, 2019.
Peng, M. et al., The Role of Clusterin in Canacer Metastasis, Cancer Management and Research 11:2405-2414, 2019.
Taki, M. et al., Tumor Immune Microenvironment during Epithelial-Mesenchymal Transition, Clin Can Res., 27:4669-4679, 2021.
International application No. PCT/CA2006/001505 filed on Sep. 13, 2006 in the name of National Research Council of Canada and published Mar. 22, 2007, under No. WO2007/030930 A1.
International application No. PCT/CA2021/050572 filed on Apr. 27, 2021, in the name of Alethia Biotherapeutics Inc. and published on Nov. 3, 2022, under No. WO2022/226623 A1.
International application No. PCT/CA2022/050632 filed on Apr. 26, 2022, in the name of Alethia Biotherapeutics Inc. and published on Nov. 3, 2022, under No. WO2022/226637 A1.
Alethia Biotherapeutics Inc. Clinical Trial Protocol entitled “AB-16B5 Combined With Docetaxel in Subjects With Metastatic Non-Small Cell Lung Cancer” identifier number: NTC043634620 published on clinicaltrials.gov on Apr. 28, 2020.
De Petris, L et al., Docetaxel Administered Every Two Weeks as Second-line Chemotherapy for Advanced Non-small Cell Lung Cancer: A Phase II Study, Anticancer Research November 2005, 25:4713-4717.

TABLE 3
SEQUENCE LISTING TABLE

| | | SEQ ID |
|---|---|---|
| ID | SEQUENCE | NO: |
| 16B5 CDRL1 | KSSQSLLNSRTRKNYLA | 1 |
| 16B5 CDRL2 | WASTRES | 2 |
| 16B5 CDRL3 | KQSYNLWT | 3 |
| 16B5 CDRH1_v1 | GFNIKDIYMH | 4 |
| 16B5 CDRH2_v1 | RIDPAYGNTKYDPKFQG | 5 |
| 16B5 CDRH3_v1 | RYDTAMDY | 6 |
| 16B5 CDRH1_v2 | GFNIKDIY | 7 |
| 16B5 CDRH2_v2 | IDPAYGNT | 8 |
| 16B5 CDRH3_v2 | ARRYDTAMDY | 9 |
| murine 16B5 VL | DIVMSQSPSSLAVSAGEKVTMSCKSSQSLLNSRTRKNY | 10 |
| | LAWYQQKPGQSPKLLIYWASTRESGVPDRFTGSGSGTD | |
| | FTLTISSVQAEDLAVYYCKQSYNLWTFGGGTKLEFK | |
| murine 16B5 VH | EVQLQQSGAELVKPGASVRLSCTTSGFNIKDIYMHWVK | 11 |
| | QRPEQGLEWIGRIDPAYGNTKYDPKFQGKATITADTSS | |
| | NTAYLQLSSLTSEDTAVYYCARRYDTAMDYWGQGTSVT | |
| | VSS | |
| Humanized 16B5 | DIVMTQSPDSLAVSLGERATINCKSSQSLLNSRTRKNY | 12 |
| VL | LAWYQQKPGQPPKLLIYWASTRESGVPDRFSGSGSGTD | |
| | FTLTISSLQAEDVAVYYCKQSYNLWTFGQGTKLEIK | |
| Humanized 16B5 | QVQLVQSGAEVKKPGATVKISCKVSGFNIKDIYMHWVQ | 13 |
| VH | QAPGKGLEWMGRIDPAYGNTKYDPKFQGRVTITADTST | |
| | DTAYMELSSLRSEDTAVYYCARRYDTAMDYWGQGTLVT | |
| | VSS | |
| Humanized 16B5 | DIVMTQSPDSLAVSLGERATINCKSSQSLLNSRTRKNY | 14 |
| light chain | LAWYQQKPGQPPKLLIYWASTRESGVPDRFSGSGSGTD | |
| | FTLTISSLQAEDVAVYYCKQSYNLWTFGQGTKLEIKVA | |
| | APSVFIFPPSDEQLKSGTASVVCLLNNFYPREAKVQWK | |
| | VDNALQSGNSQESVTEQDSKDSTYSLSSTLTLSKADYE | |
| | KHKVYACEVTHQGLSSPVTKSFNRGEC | |
| Humanized 16B5 | QVQLVQSGAEVKKPGATVKISCKVSGFNIKDIYMHWVQ | 15 |
| heavy chain | QAPGKGLEWMGRIDPAYGNTKYDPKFQGRVTITADTST | |
| | DTAYMELSSLRSEDTAVYYCArRYDTAMDYwgqgtlvt | |
| | vsSASTKGPSVFPLAPCSRSTSESTAALGCLVKDYFPE | |
| | PVTVSWNSGALTSGVHTFPAVLQSSGLYSLSSVVTVPS | |
| | SNFGTQTYTCNVDHKPSNTKVDKTVERKCCVECPPCPA | |
| | PPVAGPSVFLFPPKPKDTLMISRTPEVTCVVVDVSHED | |
| | PEVQFNWYVDGVEVHNAKTKPREEQFNSTFRVVSVLTV | |
| | VHQDWLNGKEYKCKVSNKGLPAPIEKTISKTKGQPREP | |
| | QVYTLPPSREEMTKNQVSLTCLVKGFYPSDIAVEWESN | |
| | GQPENNYKTTPPMLDSDGSFFLYSKLTVDKSRWQQGNV | |
| | FSCSVMHEALHNHYTQKSLSLSPGK | |
| Humanized 16B5 | ATGGTGCTGCAGACCCAGGTGTTCATCTCCCTGCTGCT | 16 |
| light chain | GTGGATCTCCGGCGCCTACGGCGACATCGTGATGACCC | |
| nucleic acid | AGTCCCCCGACTCCCTGGCCGTGTCCCTGGGCGAGAGA | |
| | GCCACCATCAACTGCAAGTCCTCCCAGTCCCTGCTGAA | |
| | CTCCCGGACCCGGAAGAACTACCTGGCCTGGTATCAGC | |
| | AGAAGCCTGGCCAGCCTCCTAAGCTGCTGATCTACTGG | |
| | GCCTCCACCCGGGAGTCCGGCGTGCCTGACCGGTTCTC | |
| | CGGCTCCGGCAGCGGCACCGACTTCACCCTGACCATCA | |
| | GCTCCCTGCAGGCCGAGGACGTGGCCGTGTACTACTGC | |
| | AAGCAGTCCTACAACCTGTGGACCTTCGGCCAGGGCAC | |
| | CAAGCTGGAGATCAAGCGGACTGTGGCTGCACCATCTG | |
| | TCTTCATCTTCCCGCCATCTGATGAGCAGTTGAAATCT | |
| | GGAACTGCCTCTGTTGTGTGCCTGCTGAATAACTTCTA | |
| | TCCCAGAGAGGCCAAAGTACAGTGGAAGGTGGATAACG | |
| | CCCTCCAATCGGGTAACTCCCAGGAGAGTGTCACAGAG | |
| | CAGGACAGCAAGGACAGCACCTACAGCCTCAGCAGCAC | |
| | CCTGACGCTGAGCAAAGCAGACTACGAGAAACACAAAG | |
| | TCTACGCCTGCGAAGTCACCCATCAGGGCCTGAGCTCG | |
| | CCCGTCACAAAGAGCTTCAACAGGGGAGAGTGTTAG | |
| Humanized 16B5 | ATGGACTGGACCTGGCGGATCCTGTTCCTGGTGGCCGC | 17 |
| heavy chain | TGCTACCGGCACCCACGCCCAGGTGCAGCTGGTGCAGT | |
| nucleic acid | CTGGCGCCGAGGTGAAGAAGCCTGGCGCCACCGTCAAG | |
| | ATCAGCTGCAAGGTGTCCGGCTTCAACATCAAGGACAT | |
| | CTACATGCACTGGGTGCAGCAGGCTCCAGGCAAGGGAC | |
| | TGGAGTGGATGGGCCGGATCGACCCTGCCTACGGCAAC | |
| | ACCAAGTACGACCCTAAGTTCCAGGGCCGGGTGACCAT | |
| | CACCGCCGACACCTCCACCGACACCGCCTACATGGAAC | |
| | TGTCCTCCCTGCGGTCCGAGGACACCGCCGTGTACTAC | |
| | TGCGCCCGGAGATACGACACCGCCATGGATTACTGGGG | |
| | CCAGGGCACCCTGGTGACCGTGTCCTCCGCTTCCACCA | |
| | AGGGCCCATCGGTCTTCCCCCTGGCGCCCTGCTCCAGG | |
| | AGCACCTCCGAGAGCACAGCGGCCCTGGGCTGCCTGGT | |
| | CAAGGACTACTTCCCCGAACCGGTGACGGTGTCGTGGA | |
| | ACTCAGGCGCTCTGACCAGCGGCGTGCACACCTTCCCA | |
| | GCTGTCCTACAGTCCTCAGGACTCTACTCCCTCAGCAG | |
| | CGTGGTGACCGTGCCCTCCAGCAACTTCGGCACCCAGA | |
| | CCTACACCTGCAACGTAGATCACAAGCCCAGCAACACC | |
| | AAGGTGGACAAGACAGTTGAGCGCAAATGTTGTGTCGA | |
| | GTGCCCACCGTGCCCAGCACCACCTGTGGCAGGACCGT | |
| | CAGTCTTCCTCTTCCCCCCAAAACCCAAGGACACCCTC | |
| | ATGATCTCCCGGACCCCTGAGGTCACGTGCGTGGTGGT | |
| | GGACGTGAGCCACGAAGACCCCGAGGTCCAGTTCAACT | |
| | GGTACGTGGACGGCGTGGAGGTGCATAATGCCAAGACA | |
| | AAGCCACGGGAGGAGCAGTTCAACAGCACGTTCCGTGT | |
| | GGTCAGCGTCCTCACCGTTGTGCACCAGGACTGGCTGA | |
| | ACGGCAAGGAGTACAAGTGCAAGGTCTCCAACAAAGGC | |
| | CTCCCAGCCCCCATCGAGAAAACCATCTCCAAAACCAA | |
| | AGGGCAGCCCCGAGAACCACAGGTGTACACCCTGCCCC | |
| | CATCCCGGGAGGAGATGACCAAGAACCAGGTCAGCCTG | |
| | ACCTGCCTGGTCAAAGGCTTCTACCCCAGCGACATCGC | |
| | CGTGGAGTGGGAGAGCAATGGGCAGCCGGAGAACAACT | |
| | ACAAGACCACACCTCCCATGCTGGACTCCGACGGCTCC | |
| | TTCTTCCTCTACAGCAAGCTCACCGTGGACAAGAGCAG | |
| | GTGGCAGCAGGGGAACGTCTTCTCATGCTCCGTGATGC | |
| | ATGAGGCTCTGCACAACCACTACACGCAGAAGAGCCTC | |
| | TCCCTGTCTCCGGGTAAATGA | |
| Amino acids 421 | LTQGEDQYYLRVTTVASHTSDSDVPSGVTEVVVKLFDS | 18 |
| and 443 of a C- | DPITVTVPVEVSRKNPKFMETVAEKALQEYRKKHREE | |
| terminal portion | | |
| of a β-subunit of | | |
| human clusterin | | |
| 21B12 CDRL1 | KSSQSLLYSSNQKNYLA | 19 |
| 21B12 CDRL2 | WASTRES | 20 |
| 21B12 CDRL3 | QQYYIYPRT | 21 |
| 21B12 | GYTFTNYGMH | 22 |
| CDRH1_v1 | | |
| 21B12 | WINTYTGEPTYADDFKG | 23 |
| CDRH2_v1 | | |
| 21B12 | DGFLYFFDY | 24 |
| CDRH3_v1 | | |
| 21B12 | GYTFTNYG | 25 |
| CDRH1_v2 | | |
| 21B12 | INTYTGEP | 26 |
| CDRH2_v2 | | |
| 21B12 | DGFLYFFDY | 27 |
| CDRH3_v2 | | |
| Murine 21B12 | DIVMSQSPSSLAVSVGEKVTMSCKSSQSLLYSSNQKNY | 28 |
| VL | LAWYQQRPGQSPKLLIYWASTRESGVPDRFTGSGSGTD | |
| | FTLTISSVKAEDLAVYYCQQYYIYPRTFGGGTKLEIK | |
| Murine 21B12 | QIQLVQSGPELKKPGETVKISCKASGYTFTNYGMHWVK | 29 |
| VH | QAPGKGLKWMGWINTYTGEPTYADDFKGRFAFSLETSA | |
| | STAYLQINNLKNEDTATYFCARDGFLYFFDYWGQGTTL | |
| | TVSS | |
| Humanized | DIVMTQSPDSLAVSLGERATINCKSSQSLLYSSNQKNY | 30 |
| 21B12 VL | LAWYQQKPGQPPKLLIYWASTRESGVPDRFSGSGSGTD | |
| | FTLTISSLQAEDVAVYYCQQYYIYPRTFGQGTKLEIK | |
| Humanized | QVQLVQSGSELKKPGASVKVSCKASGYTFTNYGMHWVR | 31 |
| 21B12 VH | QAPGQGLEWMGWINTYTGEPTYADDFKGRFVFSLDTSV | |
| | STAYLQISSLKAEDTAVYYCARdGFLYFFDYwgqgtlv | |
| | tvss | |
| Humanized | DIVMTQSPDSLAVSLGERATINCKSSQSLLYSSNQKNY | 32 |
| 21B12 light chain | LAWYQQKPGQPPKLLIYWASTRESGVPDRFSGSGSGTD | |
| | FTLTISSLQAEDVAVYYCQQYYIYPRTFGQGTKLEIKR | |
| | TVAAPSVFIFPPSDEQLKSGTASVVCLLNNFYPREAKV | |
| | QWKVDNALQSGNSQESVTEQDSKDSTYSLSSTLTLSKA | |
| | DYEKHKVYACEVTHQGLSSPVTKSFNRGEC | |
| Humanized | QVQLVQSGSELKKPGASVKVSCKASGYTFTNYGMHWVR | 33 |
| 21B12 heavy | QAPGQGLEWMGWINTYTGEPTYADDFKGRFVFSLDTSV | |
| chain | STAYLQISSLKAEDTAVYYCARdGFLYFFDYWGQGTLV | |
| | TVSSASTKGPSVFPLAPCSRSTSESTAALGCLVKDYFP | |
| | EPVTVSWNSGALTSGVHTFPAVLQSSGLYSLSSVVTVP | |
| | SSNFGTQTYTCNVDHKPSNTKVDKTVERKCCVECPPCP | |
| | APPVAGPSVFLFPPKPKDTLMISRTPEVTCVVVDVSHE | |
| | DPEVQFNWYVDGVEVHNAKTKPREEQFNSTFRVVSVLT | |
| | VVHQDWLNGKEYKCKVSNKGLPAPIEKTISKTKGQPRE | |
| | PQVYTLPPSREEMTKNQVSLTCLVKGFYPSDIAVEWES | |
| | NGQPENNYKTTPPMLDSDGSFFLYSKLTVDKSRWQQGN | |
| | VFSCSVMHEALHNHYTQKSLSLSPGK | |
| Humanized | ATGGTGCTGCAGACCCAGGTGTTCATCTCCCTGCTGCT | 34 |
| 21B12 light chain | GTGGATCTCTGGCGCCTACGGCGACATCGTGATGACCC | |
| nucleic acid | AGTCCCCCGACTCTCTGGCTGTGTCCCTGGGCGAGCGG | |
| | GCCACCATCAACTGCAAGTCCTCCCAGTCCCTGCTGTA | |
| | CTCCTCCAACCAGAAGAACTACCTGGCCTGGTATCAGC | |
| | AGAAGCCTGGCCAGCCTCCTAAGCTGCTGATCTACTGG | |
| | GCCTCCACCCGGGAATCTGGCGTGCCTGACCGGTTCTC | |
| | CGGCTCTGGCTCCGGCACCGACTTCACCCTGACCATCA | |
| | GCTCCCTGCAGGCCGAGGACGTGGCCGTGTACTACTGC | |
| | CAGCAGTACTACATCTACCCTCGGACCTTCGGCCAGGG | |
| | CACCAAGCTGGAAATCAAGCGGACCGTGGCCGCTCCTT | |
| | CCGTGTTCATCTTCCCCCCTTCCGACGAGCAGCTGAAG | |
| | TCCGGCACCGCCTCTGTGGTGTGCCTGCTGAACAACTT | |
| | CTACCCCCGGGAGGCCAAGGTGCAGTGGAAGGTGGACA | |
| | ACGCCCTGCAGTCCGGCAACTCCCAGGAATCCGTCACC | |
| | GAGCAGGACTCCAAGGACTCTACCTACTCCCTGTCCTC | |
| | CACCCTGACCCTGTCCAAGGCCGACTACGAGAAGCACA | |
| | AGGTGTACGCCTGCGAAGTGACCCACCAGGGCCTGTCC | |
| | TCTCCCGTGACCAAGTCCTTCAACCGGGGCGAGTGCTG | |
| | A | |
| Humanized | ATGGACTGGACCTGGCGGATCCTGTTTCTGGTGGCCGC | 35 |
| 21B12 heavy | TGCTACCGGCACACACGCCCAGGTGCAGCTGGTGCAGT | |
| chain nucleic acid | CCGGCTCCGAGCTGAAGAAACCTGGCGCCTCCGTGAAG | |
| | GTGTCCTGCAAGGCCTCCGGCTACACCTTCACCAACTA | |
| | CGGCATGCACTGGGTGCGCCAGGCACCTGGACAGGGAC | |
| | TGGAATGGATGGGCTGGATCAACACCTACACCGGCGAG | |
| | CCTACCTACGCCGACGACTTCAAGGGCAGATTCGTGTT | |
| | CTCCCTGGACACCTCCGTGTCCACCGCCTACCTGCAGA | |
| | TCTCCTCCCTGAAGGCCGAGGACACCGCCGTGTACTAC | |
| | TGCGCCAGGGACGGCTTCCTGTACTTCTTCGACTACTG | |
| | GGGCCAGGGCACCCTGGTGACCGTGTCCTCTGCCTCCA | |
| | CCAAGGGCCCTTCCGTGTTCCCTCTGGCCCCTTGCTCC | |
| | CGGTCCACCTCTGAGTCTACCGCCGCTCTGGGCTGCCT | |
| | GGTGAAGGACTACTTCCCTGAGCCTGTGACAGTGTCCT | |
| | GGAACTCTGGCGCCCTGACCTCTGGCGTGCACACCTTC | |
| | CCTGCCGTGCTGCAGTCCTCCGGCCTGTACTCCCTGTC | |
| | CTCCGTGGTGACAGTGCCTTCCTCCAACTTCGGCACCC | |
| | AGACCTACACCTGCAACGTGGACCACAAGCCTTCCAAC | |
| | ACCAAGGTGGACAAGACCGTGGAGCGGAAGTGCTGCGT | |
| | GGAGTGCCCTCCTTGTCCTGCTCCTCCTGTGGCTGGCC | |
| | CTAGCGTGTTCCTGTTCCCTCCTAAGCCTAAGGACACC | |
| | CTGATGATCTCCCGGACCCCTGAAGTGACCTGCGTGGT | |
| | GGTGGACGTGTCCCACGAGGACCCTGAGGTGCAGTTCA | |
| | ATTGGTACGTGGACGGCGTGGAGGTGCACAACGCCAAG | |
| | ACCAAGCCTCGGGAGGAACAGTTCAACTCCACCTTCCG | |
| | GGTGGTGTCCGTGCTGACCGTGGTGCACCAGGACTGGC | |
| | TGAACGGCAAAGAATACAAGTGCAAGGTGTCCAACAAG | |
| | GGCCTGCCTGCCCCTATCGAAAAGACCATCTCTAAGAC | |
| | CAAGGGCCAGCCTCGCGAGCCTCAGGTGTACACCCTGC | |
| | CTCCCTCCCGCGAGGAAATGACCAAGAACCAGGTGTCC | |
| | CTGACCTGTCTGGTGAAGGGCTTCTACCCTTCCGATAT | |
| | CGCCGTGGAGTGGGAGTCTAACGGCCAGCCTGAGAACA | |
| | ACTACAAGACCACCCCTCCTATGCTGGACTCCGACGGC | |
| | TCCTTCTTCCTGTACAGCAAGCTGACAGTGGACAAGTC | |
| | CCGGTGGCAGCAGGGCAACGTGTTCTCCTGCTCCGTGA | |
| | TGCACGAGGCCCTGCACAACCACTACACCCAGAAGTCC | |
| | CTGTCCCTGTCTCCTGGCAAGTGA | |
| 20E11 variable | DIVLTLSPASLAVSLGQRATISCRASQSVNSSNYSYMH | 36 |
| light chain | WYQQKPGQPPKLLIKYASNLESGVPARFSGSGSGTHFT | |
| | LNIHPVEEEDTATYYCQHSWEIPWTFGGGTKLEIK | |
| 20E11 variable | QIQLVQSGPELKKPGETVKISCKASGYTFTDYSMHWVK | 37 |
| heavy chain | QAPGKGLKWMGWINTETGEPTYADDFKGRFAFSLETSA | |
| | STAYLQINNLKNEDTATYFCARTGSSGYFDCWGQGTTL | |
| | TVSS | |
| 11E2 variable | ENVLTQSPAIMSASPGEKVTMTCSASSSVSYMHWYQQK | 38 |
| light chain Gr1 | SSTSPKLWIYDTSKLASGVPGRFSGSGSGNSYSLTISS | |
| | MEAEDVATYYCFQGSGYPFTFGSGTKLEIK | |
| 11E2 variable | DIQMTQSPSSLSASLGGKVTITCKASQDINKYIAWYQH | 39 |
| light chain GR2 | KPGKGPRLLIHYTSTLQPGIPSRFSGSGSGRDYSFSIS | |
| | NLEPEDIATYYCLQYDNLLRTFGGGTKLEIK | |
| 11E2 variable | EVQLQQSGPELEKPGASVKISCKASGYSFTGYNMNWVK | 40 |
| heavy chain | QNNGKSLEWIGNIDPYYGTPNYNQKFKGKATLTVDKSS | |
| | STAYMQLKSLTSEDSAVYYCALNSLLRLNAMDYWGQGT | |
| | SVTVSS | |
| 16C11 variable | EVQLQQSGPELGKPGASVKISCKASGYSFTGYNMYWVK | 41 |
| heavy chain | QSHRKSLEWIGYIDPYNGDTSYNQKSKGKATLTADRSS | |
| | STAYMHLNSLTSEDSGIYYCARGAYGSSYAYWGQGTLV | |
| | AVSA | |

1. A method of treating a subject having non-small cell lung cancer (NSCLC), the method comprising administering to the subject a combination therapy comprising an anti-clusterin antibody or antigen binding fragment thereof and a taxane.

2. The method of claim 1, wherein the taxane is docetaxel or paclitaxel.

3. The method of claim 1 or 2, wherein the anti-clusterin antibody or antigen binding fragment thereof is administered at a dose of between approximately 3 mg/kg to approximately 20 mg/kg.

4. The method of any one of claims 1 to 3, wherein the taxane is docetaxel and is administered at a dose of between approximately 50 mg/m² to approximately 100 mg/m².

5. The method of any one of the preceding claims, wherein the combination therapy is administered as a therapeutically effective combination therapy.

6. The method of any one of the preceding claims, wherein the method comprises administering the anti-clusterin antibody or antigen binding fragment thereof at a dose of between approximately 3 mg/kg to approximately 20 mg/kg once per week, once every two weeks or once every three weeks and administering docetaxel at a dose of between approximately 50 mg/m² to approximately 100 mg/m² once every two weeks or once every three weeks.

7. The method of any one of the preceding claims, wherein the anti-clusterin antibody or antigen binding fragment thereof is administered at a dose of approximately 3 mg/kg.

8. The method of any one of the preceding claims, wherein the anti-clusterin antibody or antigen binding fragment thereof is administered at a dose of approximately 6 mg/kg.

9. The method of any one of the preceding claims, wherein the anti-clusterin antibody or antigen binding fragment thereof is administered at a dose of approximately 9 mg/kg.

10. The method of any one of the preceding claims, wherein the anti-clusterin antibody or antigen binding fragment thereof is administered at a dose of approximately 12 mg/kg.

11. The method of any one of the preceding claims, wherein the taxane is docetaxel and is administered at a dose of approximately 50 mg/m².

12. The method of any one of the preceding claims, wherein the taxane is docetaxel and is administered at a dose of approximately 60 mg/m².

13. The method of any one of the preceding claims, wherein the taxane is docetaxel and is administered at a dose of approximately 75 mg/m².

14. The method of any one of the preceding claims, wherein the taxane is docetaxel and is administered at a dose of approximately 100 mg/m².

15. The method of any one of the preceding claims, wherein the anti-clusterin antibody or antigen binding fragment thereof is administered once weekly.

16. The method of any one of the preceding claims, wherein the anti-clusterin antibody or antigen binding fragment thereof is administered once every two weeks.

17. The method of any one of the preceding claims, wherein the anti-clusterin antibody or antigen binding fragment thereof is administered once every three weeks.

18. The method of any one of the preceding claims, wherein docetaxel is administered once every two weeks.

19. The method of any one of the preceding claims, wherein docetaxel is administered once every three weeks.

20. The method of any one of the preceding claims, wherein the method comprises administering at least one initial cycle of treatment and at least one subsequent cycle of treatment, wherein the initial cycle of treatment and subsequent cycle of treatment are identical or different and each comprise at least one dose of the anti-clusterin antibody or antigen binding fragment thereof and at least one dose of docetaxel.

21. The method of claim 20, wherein the initial cycle of treatment and/or subsequent cycle of treatment each independently comprises administering the anti-clusterin antibody or antigen binding fragment thereof at a dose of approximately 3 mg/kg to approximately 20 mg/kg once per week, once every two weeks, or once every three weeks and administration of docetaxel at a dose of approximately 50 mg/m² to 100 mg/m² once every two weeks or once every three weeks.

22. The method of any one of claims 1 to 21, wherein the method comprises administering docetaxel at a dose of approximately 75 mg/m² once every three weeks and administering the anti-clusterin antibody or antigen binding fragment thereof per week, once every two weeks or once every three weeks.

23. The method of any one of claims 1 to 22, wherein the method comprises administering the anti-clusterin antibody or antigen binding fragment thereof at a dose of approximately 12 mg/kg once per week and administering docetaxel at a dose of approximately 75 mg/m² once every three weeks.

24. The method of any one of claims 1 to 23, wherein the method comprises administering the anti-clusterin antibody or antigen binding fragment thereof at a dose of approximately 12 mg/kg once every three weeks and administering docetaxel at a dose of approximately 75 mg/m² once every three weeks.

25. The method of any one of claims 1 to 24, wherein the method comprises administering:
a. at least one initial cycle of treatment comprising administering the anti-clusterin antibody or antigen binding fragment thereof at a dose of approximately 12 mg/kg once per week and administration of docetaxel at a dose of approximately 75 mg/m² once every three weeks and;
b. at least one subsequent cycle of treatment comprising administering the anti-clusterin antibody or antigen binding fragment thereof at a dose of approximately 3 mg/kg to approximately 20 mg/kg once every three weeks and administration of docetaxel at a dose of approximately 75 mg/m² once every three weeks.

26. The method of any one of claims 1 to 25, wherein the method comprises administering:
a. at least one initial cycle of treatment comprising administering the anti-clusterin antibody or antigen binding fragment thereof at a dose of approximately 12 mg/kg once per week and administration of docetaxel at a dose of approximately 75 mg/m² once every three weeks and;
b. at least one subsequent cycle of treatment comprising administering the anti-clusterin antibody or antigen binding fragment thereof at a dose of approximately 12 mg/kg once every three weeks and administration of docetaxel at a dose of approximately 75 mg/m² once every three weeks.

27. The method of any one of claims 1 to 26, wherein the method comprises administering docetaxel at a dose of approximately 50 mg/m² once every two weeks and administering the anti-clusterin antibody or antigen binding fragment thereof once per week, once every two weeks or once every three weeks.

28. The method of any one of claims 1 to 27, wherein the method comprises administering the anti-clusterin antibody or antigen binding fragment thereof at a dose of approximately 12 mg/kg once per week and administering docetaxel at a dose of approximately 50 mg/m² once every two weeks.

29. The method of any one of claims 1 to 28, wherein the method comprises administering the anti-clusterin antibody or antigen binding fragment thereof at a dose of approximately 12 mg/kg once every two weeks and administering docetaxel at a dose of approximately 50 mg/m² once every two weeks.

30. The method of any one of claims 1 to 29, wherein the method comprises administering:
a. at least one initial cycle of treatment comprising administering the anti-clusterin antibody or antigen binding fragment thereof at a dose of approximately 12 mg/kg once per week and administration of docetaxel at a dose of approximately 50 mg/m² once every two weeks and;
b. at least one subsequent cycle of treatment comprising administering the anti-clusterin antibody or antigen binding fragment thereof at a dose of approximately 3 mg/kg to approximately 20 mg/kg once every two weeks and administration of docetaxel at a dose of approximately 50 mg/m² once every two weeks.

31. The method of any one of claims 1 to 30, wherein the method comprises administering:
a. at least one initial cycle of treatment comprising administering the anti-clusterin antibody or antigen binding fragment thereof at a dose of approximately 12 mg/kg once per week and administration of docetaxel at a dose of approximately 50 mg/m² once every two weeks and;
b. at least one subsequent cycle of treatment comprising administering the anti-clusterin antibody or antigen binding fragment thereof at a dose of approximately 12 mg/kg once every two weeks and administration of docetaxel at a dose of approximately 50 mg/m² once every two weeks.

32. A method of treating non-small cell lung cancer (NSCLC) in a subject in need thereof, comprising administering a combination therapy for at least one cycle of treatment comprising administration of the anti-clusterin antibody at a dose of approximately 12 mg/kg once per week, once every two weeks, once every three weeks or once every four weeks and administration of docetaxel at a dose of between approximately 50 mg/m² to approximately 75 mg/m² once every two to three weeks, wherein the anti-clusterin antibody comprises a light chain variable region comprising the complementarity determining regions (CDRs) of the light chain variable region set forth in SEQ ID NO: 12 and a heavy chain variable region comprising the CDRs of the heavy chain variable region set forth in SEQ ID NO: 13.

33. The method of any one of the preceding claims, wherein the anti-clusterin antibody or antigen binding fragment thereof and docetaxel are administered on same day.

34. The method of any one of the preceding claims, wherein the method is carried so as to obtain and/or maintain a clinical benefit.

35. The method of any one of the preceding claims, wherein the anti-clusterin antibody or antigen binding fragment thereof is capable of binding to a C-terminal portion of a β-subunit of human clusterin.

36. The method of any one of the preceding claims, wherein the anti-clusterin antibody or antigen binding fragment thereof is capable of binding to amino acids 421 and 443 or to an epitope comprised within amino acids 421 and 443 of a C-terminal portion of a β-subunit of human clusterin.

37. The method of any one of the preceding claims, wherein the anti-clusterin antibody or antigen binding fragment thereof comprises a light chain variable region comprising the complementarity determining regions (CDRs) of the light chain variable region set forth in SEQ ID NO: 12 and a heavy chain variable region comprising the CDRs of the heavy chain variable region set forth in SEQ ID NO: 13.

38. The method of any one of the preceding claims, wherein the subject has or is selected for having NSCLC that has failed prior treatment comprising an immune checkpoint antibody with or without chemotherapy.

39. The method of any one of the preceding claims, wherein the immune checkpoint antibody is an anti-PD-1 or anti-PD-L1 immune checkpoint antibody.

40. The method of any one of the preceding claims, wherein the subject has or is selected for having NSCLC that has failed prior treatment with an anti-PD-1 or PD-L1 immune checkpoint antibody and a platinum-containing doublet treatment.

41. The method of any one of claims 38 to 40, wherein the immune checkpoint antibody is selected from ipilimumab, nivolumab, pembrolizumab, cemiplimab, atezolizumab, avelumab, or durvalumab.

42. The method of any one of the preceding embodiments, wherein the subject has or is selected for having NSCLC characterized as having a KRAS mutation.

43. The method of any one of the preceding claims, wherein the subject has or is selected for having one or more lesions with a PD-L1 tumor proportion score of ≤15%, <5%, ≤1%, or <1% or with no evidence of PD-L1 expression.

44. The method of any one of the preceding claims, wherein the subject has or is selected for having NSCLC that is not eligible for treatment comprising an immune checkpoint antibody or the subject would unlikely benefit from treatment comprising an immune checkpoint antibody.

45. The method of any one of the preceding claims, wherein the subject has or is selected for having one or more lesions characterized as having poor infiltration of immune cells or as being immunologically cold.

46. The method of any one of the preceding claims, wherein the subject has or is selected for having one or more lesions having an EMT signature or showing signs of an EMT signature.

47. The method of any one of the preceding claims, wherein one cycle of treatment lasts approximately 3 weeks.

48. The method of any one of the preceding claims, wherein the anti-clusterin antibody or antigen binding fragment thereof is administered by IV infusion.

49. The method of any one of the preceding claims, wherein docetaxel is administered by IV infusion.

50. The method of any one of the preceding claims, wherein the clinical benefit is complete response (CR) as per RECIST 1.1 guidelines.

51. The method of any one of the preceding claims, wherein the clinical benefit is partial response (PR) as per RECIST 1.1 guidelines.

52. The method of any one of the preceding claims, wherein the clinical benefit is stable disease (SD) as per RECIST 1.1 guidelines.

53. The method of any one of the preceding claims, wherein the clinical benefit is is a decrease in the size of the lesion as assessed in accordance with RECIST 1.1 guidelines.

54. The method of any one of the preceding claims, wherein the combination therapy is administered for at least 2 cycles consisting in administration of the anti-clusterin antibody once per week and administration of docetaxel once every three weeks.

55. The method of any one of the preceding claims, wherein the combination therapy is administered for at least 2 cycles, 3 cycles, at least 4 cycles, at least 5 cycles, at least 6 cycles, at least 7 cycles, at least 8 cycles, at least 9 cycles, at least 10 cycles, at least 11 cycles, at least 12 cycles, at least 13 cycles, at least 14 cycles, at least 15 cycles, at least 16 cycles, at least 17 cycles, at least 18 cycles, at least 19 cycles, at least 20 cycles, at least 21 cycles or at least 22 cycles.

56. The method of any one of the preceding claims, wherein the combination therapy is administered for at least one to at least five initial cycles of treatment.

57. The method of any one of the preceding claims, wherein the combination therapy is administered for two or more subsequent cycles of treatment.

58. The method of any of the preceding claims, wherein the anti-clusterin antibody or antigen binding fragment thereof comprises:
a. a light chain variable region having an amino acid sequence having at least 80% identity with the amino acid sequence set forth in SEQ ID NO: 12 and a heavy chain variable region having an amino acid sequence at least 80% identity with the amino acid sequence set forth in SEQ ID NO: 13 or;
b. a light chain having an amino acid sequence having at least 80% identity with the amino acid sequence set forth in SEQ ID NO:14 and a heavy chain having an amino acid sequence having at least 80% identity with the amino acid sequence set forth in SEQ ID NO:15.

59. The method of any of the preceding claims, wherein the anti-clusterin antibody or antigen binding fragment thereof comprises:
a. a light chain variable region having the amino acid sequence set forth in SEQ ID NO: 12 and a heavy chain variable region having the amino acid sequence set forth in SEQ ID NO:13 or;
b. a light chain having the amino acid sequence set forth in SEQ ID NO:14 and a heavy chain having the amino acid sequence set forth in SEQ ID NO:15.

60. The method of any one of the preceding claims, wherein the subject is not immunosuppressed or has not received an immunosuppressive medication within 7 days prior to treatment.

61. The method of any one of the preceding claims, wherein the subject is a human subject.

62. The method of any one of the preceding claims, wherein the NSCLC is metastatic NSCLC.

63. The method of any one of the preceding claims, wherein the NSCLC is stage III to IV NSCLC.

64. The method of any one of the preceding claims, wherein docetaxel is discontinued or ceased for one or more cycles of treatment upon signs of toxicity.

65. The method of claim 64, wherein administration of the anti-clusterin antibody or antigen binding fragment thereof is continued.

66. The method of any one of the preceding claims, wherein the anti-clusterin antibody or antigen binding fragment thereof is a conventional antibody.

67. A therapeutically effective combination therapy comprising an anti-clusterin antibody or antigen binding fragment thereof and a taxane for use in the treatment of non-small cell lung cancer (NSCLC) in a subject in need thereof.

68. The combination therapy of claim 67, wherein the subject in need has metastatic NSCLC.

69. The combination therapy of claim 67 or 68, wherein the combination therapy is used in the method of any one of claims 1-66.


SEQUENCE LISTING (41 sequences)

SEQ ID NO: 1 - protein, 17 aa, synthetic construct
KSSQSLLNSRTRKNYLA

SEQ ID NO: 2 - protein, 7 aa, synthetic construct
WASTRES

SEQ ID NO: 3 - protein, 8 aa, synthetic construct
KQSYNLWT

SEQ ID NO: 4 - protein, 10 aa, synthetic construct
GFNIKDIYMH

SEQ ID NO: 5 - protein, 17 aa, synthetic construct
RIDPAYGNTKYDPKFQG

SEQ ID NO: 6 - protein, 8 aa, synthetic construct
RYDTAMDY

SEQ ID NO: 7 - protein, 8 aa, synthetic construct
GFNIKDIY

SEQ ID NO: 8 - protein, 8 aa, synthetic construct
IDPAYGNT

SEQ ID NO: 9 - protein, 10 aa, synthetic construct
ARRYDTAMDY

SEQ ID NO: 10 - protein, 112 aa, synthetic construct
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYNLWTFGGGTKLEFK

SEQ ID NO: 11 - protein, 117 aa, synthetic construct
EVQLQQSGAELVKPGASVRLSCTTSGFNIKDIYMHWVKQRPEQGLEWIGRIDPAYGNTKYDPKFQGKATITADTSSNTAYLQLSSLTSEDTAVYYCARRYDTAMDYWGQGTSVTVSS

SEQ ID NO: 12 - protein, 112 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCKQSYNLWTFGQGTKLEIK

SEQ ID NO: 13 - protein, 117 aa, synthetic construct
QVQLVQSGAEVKKPGATVKISCKVSGFNIKDIYMHWVQQAPGKGLEWMGRIDPAYGNTKYDPKFQGRVTITADTSTDTAYMELSSLRSEDTAVYYCARRYDTAMDYWGQGTLVTVSS

SEQ ID NO: 14 - protein, 217 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCKQSYNLWTFGQGTKLEIKVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 443 aa, synthetic construct
QVQLVQSGAEVKKPGATVKISCKVSGFNIKDIYMHWVQQAPGKGLEWMGRIDPAYGNTKYDPKFQGRVTITADTSTDTAYMELSSLRSEDTAVYYCARRYDTAMDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 16 - DNA, 720 bp, synthetic construct
  source 1..720: mol_type = other DNA
atggtgctgcagacccaggtgttcatctccctgctgctgtggatctccggcgcctacggcgacatcgtgatgacccagtcccccgactccctggccgtgtccctgggcgagagagccaccatcaactgcaagtcctcccagtccctgctgaactcccggacccggaagaactacctggcctggtatcagcagaagcctggccagcctcctaagctgctgatctactgggcctccacccgggagtccggcgtgcctgaccggttctccggctccggcagcggcaccgacttcaccctgaccatcagctccctgcaggccgaggacgtggccgtgtactactgcaagcagtcctacaacctgtggaccttcggccagggcaccaagctggagatcaagcggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 17 - DNA, 1389 bp, synthetic construct
  source 1..1389: mol_type = other DNA
atggactggacctggcggatcctgttcctggtggccgctgctaccggcacccacgcccaggtgcagctggtgcagtctggcgccgaggtgaagaagcctggcgccaccgtcaagatcagctgcaaggtgtccggcttcaacatcaaggacatctacatgcactgggtgcagcaggctccaggcaagggactggagtggatgggccggatcgaccctgcctacggcaacaccaagtacgaccctaagttccagggccgggtgaccatcaccgccgacacctccaccgacaccgcctacatggaactgtcctccctgcggtccgaggacaccgccgtgtactactgcgcccggagatacgacaccgccatggattactggggccagggcaccctggtgaccgtgtcctccgcttccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 18 - protein, 75 aa, synthetic construct
LTQGEDQYYLRVTTVASHTSDSDVPSGVTEVVVKLFDSDPITVTVPVEVSRKNPKFMETVAEKALQEYRKKHREE

SEQ ID NO: 19 - protein, 17 aa, synthetic construct
KSSQSLLYSSNQKNYLA

SEQ ID NO: 20 - protein, 7 aa, synthetic construct
WASTRES

SEQ ID NO: 21 - protein, 9 aa, synthetic construct
QQYYIYPRT

SEQ ID NO: 22 - protein, 10 aa, synthetic construct
GYTFTNYGMH

SEQ ID NO: 23 - protein, 17 aa, synthetic construct
WINTYTGEPTYADDFKG

SEQ ID NO: 24 - protein, 9 aa, synthetic construct
DGFLYFFDY

SEQ ID NO: 25 - protein, 8 aa, synthetic construct
GYTFTNYG

SEQ ID NO: 26 - protein, 8 aa, synthetic construct
INTYTGEP

SEQ ID NO: 27 - protein, 9 aa, synthetic construct
DGFLYFFDY

SEQ ID NO: 28 - protein, 113 aa, synthetic construct
DIVMSQSPSSLAVSVGEKVTMSCKSSQSLLYSSNQKNYLAWYQQRPGQSPKLLIYWASTRESGVPDRFTGSGSGTDFTLTISSVKAEDLAVYYCQQYYIYPRTFGGGTKLEIK

SEQ ID NO: 29 - protein, 118 aa, synthetic construct
QIQLVQSGPELKKPGETVKISCKASGYTFTNYGMHWVKQAPGKGLKWMGWINTYTGEPTYADDFKGRFAFSLETSASTAYLQINNLKNEDTATYFCARDGFLYFFDYWGQGTTLTVSS

SEQ ID NO: 30 - protein, 113 aa, synthetic construct
DIVMTQSPDSLAVSLGERATINCKSSQSLLYSSNQKNYLAWYQQKPGQPPKLLIYWASTRESGVPDRFSGSGSGTDFTLTISSLQAEDVAVYYCQQYYIYPRTFGQGTKLEIK

SEQ ID NO: 31 - protein, 118 aa, synthetic construct
QVQLVQSGSELKKPGASVKVSCKASGYTFTNYGMHWVRQAPGQGLEWMGWINTYTGEPTYADDFKGRFVFSLDTSVSTAYLQISSLKAEDTAVYYCARDGFLYFFDYWGQGTLVTVSS

SEQ ID NO: 32 - protein, 220 aa, synthetic construct
DIVMTQSPDSLAVSLGERATINCKSSQSLLYSSNQKNYLAWYQQKPGQPPKLLIYWASTRESGVPDRFSGSGSGTDFTLTISSLQAEDVAVYYCQQYYIYPRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 444 aa, synthetic construct
QVQLVQSGSELKKPGASVKVSCKASGYTFTNYGMHWVRQAPGQGLEWMGWINTYTGEPTYADDFKGRFVFSLDTSVSTAYLQISSLKAEDTAVYYCARDGFLYFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 34 - DNA, 723 bp, synthetic construct
  source 1..723: mol_type = other DNA
atggtgctgcagacccaggtgttcatctccctgctgctgtggatctctggcgcctacggcgacatcgtgatgacccagtcccccgactctctggctgtgtccctgggcgagcgggccaccatcaactgcaagtcctcccagtccctgctgtactcctccaaccagaagaactacctggcctggtatcagcagaagcctggccagcctcctaagctgctgatctactgggcctccacccgggaatctggcgtgcctgaccggttctccggctctggctccggcaccgacttcaccctgaccatcagctccctgcaggccgaggacgtggccgtgtactactgccagcagtactacatctaccctcggaccttcggccagggcaccaagctggaaatcaagcggaccgtggccgctccttccgtgttcatcttccccccttccgacgagcagctgaagtccggcaccgcctctgtggtgtgcctgctgaacaacttctacccccgggaggccaaggtgcagtggaaggtggacaacgccctgcagtccggcaactcccaggaatccgtcaccgagcaggactccaaggactctacctactccctgtcctccaccctgaccctgtccaaggccgactacgagaagcacaaggtgtacgcctgcgaagtgacccaccagggcctgtcctctcccgtgaccaagtccttcaaccggggcgagtgctga

SEQ ID NO: 35 - DNA, 1392 bp, synthetic construct
  source 1..1392: mol_type = other DNA
atggactggacctggcggatcctgtttctggtggccgctgctaccggcacacacgcccaggtgcagctggtgcagtccggctccgagctgaagaaacctggcgcctccgtgaaggtgtcctgcaaggcctccggctacaccttcaccaactacggcatgcactgggtgcgccaggcacctggacagggactggaatggatgggctggatcaacacctacaccggcgagcctacctacgccgacgacttcaagggcagattcgtgttctccctggacacctccgtgtccaccgcctacctgcagatctcctccctgaaggccgaggacaccgccgtgtactactgcgccagggacggcttcctgtacttcttcgactactggggccagggcaccctggtgaccgtgtcctctgcctccaccaagggcccttccgtgttccctctggccccttgctcccggtccacctctgagtctaccgccgctctgggctgcctggtgaaggactacttccctgagcctgtgacagtgtcctggaactctggcgccctgacctctggcgtgcacaccttccctgccgtgctgcagtcctccggcctgtactccctgtcctccgtggtgacagtgccttcctccaacttcggcacccagacctacacctgcaacgtggaccacaagccttccaacaccaaggtggacaagaccgtggagcggaagtgctgcgtggagtgccctccttgtcctgctcctcctgtggctggccctagcgtgttcctgttccctcctaagcctaaggacaccctgatgatctcccggacccctgaagtgacctgcgtggtggtggacgtgtcccacgaggaccctgaggtgcagttcaattggtacgtggacggcgtggaggtgcacaacgccaagaccaagcctcgggaggaacagttcaactccaccttccgggtggtgtccgtgctgaccgtggtgcaccaggactggctgaacggcaaagaatacaagtgcaaggtgtccaacaagggcctgcctgcccctatcgaaaagaccatctctaagaccaagggccagcctcgcgagcctcaggtgtacaccctgcctccctcccgcgaggaaatgaccaagaaccaggtgtccctgacctgtctggtgaagggcttctacccttccgatatcgccgtggagtgggagtctaacggccagcctgagaacaactacaagaccacccctcctatgctggactccgacggctccttcttcctgtacagcaagctgacagtggacaagtcccggtggcagcagggcaacgtgttctcctgctccgtgatgcacgaggccctgcacaaccactacacccagaagtccctgtccctgtctcctggcaagtga

SEQ ID NO: 36 - protein, 111 aa, synthetic construct
DIVLTLSPASLAVSLGQRATISCRASQSVNSSNYSYMHWYQQKPGQPPKLLIKYASNLESGVPARFSGSGSGTHFTLNIHPVEEEDTATYYCQHSWEIPWTFGGGTKLEIK

SEQ ID NO: 37 - protein, 118 aa, synthetic construct
QIQLVQSGPELKKPGETVKISCKASGYTFTDYSMHWVKQAPGKGLKWMGWINTETGEPTYADDFKGRFAFSLETSASTAYLQINNLKNEDTATYFCARTGSSGYFDCWGQGTTLTVSS

SEQ ID NO: 38 - protein, 106 aa, synthetic construct
ENVLTQSPAIMSASPGEKVTMTCSASSSVSYMHWYQQKSSTSPKLWIYDTSKLASGVPGRFSGSGSGNSYSLTISSMEAEDVATYYCFQGSGYPFTFGSGTKLEIK

SEQ ID NO: 39 - protein, 107 aa, synthetic construct
DIQMTQSPSSLSASLGGKVTITCKASQDINKYIAWYQHKPGKGPRLLIHYTSTLQPGIPSRFSGSGSGRDYSFSISNLEPEDIATYYCLQYDNLLRTFGGGTKLEIK

SEQ ID NO: 40 - protein, 120 aa, synthetic construct
EVQLQQSGPELEKPGASVKISCKASGYSFTGYNMNWVKQNNGKSLEWIGNIDPYYGTPNYNQKFKGKATLTVDKSSSTAYMQLKSLTSEDSAVYYCALNSLLRLNAMDYWGQGTSVTVSS

SEQ ID NO: 41 - protein, 118 aa, synthetic construct
EVQLQQSGPELGKPGASVKISCKASGYSFTGYNMYWVKQSHRKSLEWIGYIDPYNGDTSYNQKSKGKATLTADRSSSTAYMHLNSLTSEDSGIYYCARGAYGSSYAYWGQGTLVAVSA